(19) United States
(12) Patent Application Publication
(10) Pub. No.: US 2025/0179167 A1
(43) Pub. Date: Jun. 5, 2025

(54) IL-6 INHIBITOR AS TREATMENT FOR NEPHROPATHY

(71) Applicant: YALE UNIVERSITY, New Haven, CT (US)

(72) Inventor: Julie GOODWIN, Guilford, CT (US)

(73) Assignee: YALE UNIVERSITY, New Haven, CT (US)

(21) Appl. No.: 18/840,763

(22) PCT Filed: Feb. 23, 2023
(86) PCT No.: PCT/US2023/063126

§ 371 Date: Aug. 22, 2024

Related U.S. Application Data
(60) Provisional application No. 63/313,101, filed on Feb. 23, 2022.

(51) Int. Cl. C07K 16/24; A61K 31/497; A61K 31/519; A61K 45/06; A61P 3/10; A61P 13/12; C07K 14/715; C07K 16/28; C12N 15/113

(52) U.S. Cl. CPC C07K 16/248; A61K 31/497; A61K 31/519; A61K 45/06; A61P 3/10; A61P 13/12; C07K 14/715; C07K 16/2866; C12N 15/1136; C07K 2317/76; C07K 2319/30; C12N 2310/14

(57) ABSTRACT

Methods and compositions for treating a disease or disorder (e.g., organ fibrosis, nephropathy) by administering an inhibitor of IL-6 activity are provided.

### CROSS-REFERENCE TO RELATED APPLICATIONS

This application claims priority to U.S. Provisional Application No. 63/313,101, filed Feb. 23, 2022, the disclosure of which is herein incorporated by reference in its entirety.

### GOVERNMENT FUNDING

This invention was made with government support HL131952 awarded by National Institutes of Health. The government has certain rights in the invention.

### FIELD OF THE INVENTION

The present disclosure generally relates to the use of IL-6 inhibitors for treatment of diseases and disorders such as organ fibrosis or nephropathy.

### SEQUENCE LISTING

The instant application contains a Sequence Listing which has been submitted electronically in XML file format and is hereby incorporated by reference in its entirety. Said XML copy, created on Feb. 20, 2023, is named 251609_000088_SL.xml and is 23,686 bytes in size.

### BACKGROUND

Approximately one-third of diabetic patients will develop diabetic nephropathy (DN), a leading cause of end-stage renal disease (ESRD) that causes more than 950,000 deaths globally each year^{1,2}. Over the last two decades, no new drugs have been approved to specifically prevent diabetic nephropathy or to improve kidney function³. This lack of advancement stems, in part, from poor understanding of the mechanisms of progressive kidney dysfunction. Furthermore, this knowledge gap contributes to suboptimal treatment options available for these patients. Improved understanding of mechanisms of pathogenesis of diabetic kidney disease is urgently needed to catalyze the development of novel, effective and safe therapeutics which can be targeted to the early stages of diabetes, before kidney damage becomes irreversible.

Diabetic nephropathy is characterized by excess deposition of extracellular matrix, loss of capillary networks and accumulation of fibrillary collagens, activated myofibroblasts and inflammatory cells^{4,5}. In renal fibrosis, myofibroblasts are believed to be an activated fibroblast phenotype that contributes to fibrosis^{5,6}. There are six well-reported sources of matrix-producing myofibroblasts: (1) activated residential fibroblasts, (2) differentiated pericytes, (3) recruited circulating fibrocytes, (4) those from macrophages via macrophage-to-mesenchymal transition (5) those from mesenchymal cells derived from tubular epithelial cells via epithelial-to-mesenchymal transition (EMT) and (6) those from mesenchymal cells transformed from endothelial cells (ECs) via endothelial-to-mesenchymal transition (EndMT)^{7,8}.

Among these diverse sources of matrix-producing fibroblasts, mesenchymal cells transformed from ECs via EndMT^{7,8}, are an important source of myofibroblasts in several organs, including the kidney⁹. EndMT is characterized by the loss of endothelial markers, including cluster of differentiation 31 (CD31), and acquisition of the expression of mesenchymal proteins including α-smooth muscle actin (αSMA), vimentin, and fibronectin^{7,8}.

ECs are critical contributors to the formation of new blood vessels in health and life-threatening diseases¹⁰. Disruption in the central metabolism of ECs contributes to disease phenotypes^{11,12}. Carnitine palmitoyltransferase 1a (CPT1a)-mediated fatty acid oxidation (FAO) regulates the proliferation of ECs in the stalk of sprouting vessels¹³⁻¹⁵. ECs use metabolites/precursors for epigenetic regulation of their sub-type differentiation and maintain crosstalk through metabolites released by other cell types^{10,15}. Notably, EndMT causes alteration of endothelial cell metabolism, and is an area of active investigation^{16,17}. For example, mesenchymal cells derived from EndMT reprogram their metabolism and show defective fatty acid metabolism¹⁷.

The contribution of EndMT to renal fibrosis has been analyzed in several mouse models of chronic kidney disease^{5-7,18,19}. Zeisberg et al., performed seminal experiments and reported that approximately 30-50% of fibroblasts co-expressed the EC marker CD31 along with markers of fibroblasts and myofibroblasts such as fibroblast specific protein-1 (FSP-1) and αSMA in the kidneys of mice subjected to unilateral ureteral obstruction nephropathy (UUO)¹⁹. The complete conversion from EC into mesenchymal cell types is not needed as intermediate cell types are sufficient to cause activation of profibrogenic pathways. EndMT can induce profibrogenic signaling in neighboring cells by autocrine and/or paracrine mechanisms thereby contributing to global kidney fibrosis^{6,20,21}. Thus, targeting EndMT might have therapeutic potential for the treatment of renal fibrosis^{6,19,22}.

### SUMMARY OF THE INVENTION

The glucocorticoid receptor (GR) is a nuclear hormone receptor that is expressed ubiquitously in most cell types and is important in many states of health and disease. Glucocorticoid receptors (GRs) mediate the action of steroid hormones in a variety of tissues, including the kidney. The role of glucocorticoids in cardiovascular and kidney disease is complex. Inventors of the present disclosure have identified endothelial GR as a negative regulator of vascular inflammation in models of sepsis²³ and atherosclerosis²⁵. The loss of endothelial GR can result in upregulation of the canonical Wnt signaling pathway. This pathway is also up regulated in renal fibrosis. However, whether endothelial GR contributes to the regulation of fibrogenic processes in the evolution of kidney fibrosis is not known.

Based on the work presented herein, the inventors show that mice lacking endothelial GR display accelerated renal fibrosis when subjected to both diabetic and non-diabetic conditions. This worsened fibrosis is associated with aberrant chemokine and cytokine reprogramming, augmented Wnt signaling and suppressed fatty acid oxidation. The disclosure demonstrates that endothelial GR is a key molecule involved in the regulation of fibrotic processes in the kidney.

One aspect of the present disclosure provides a method for treating a condition or a disease in a subject in need thereof. The method may comprise administering to the subject a therapeutically effective amount of an inhibitor of IL-6 activity, wherein the condition or disease is selected from organ fibrosis, nephropathy, dyslipidemia, hypertension, hyperlipidemia, hypercholesterolemia, cardiovascular disease, peripheral artery disease, atherosclerosis, coronary artery disease, coronary heart disease, and stroke. In one embodiment, the organ fibrosis is renal fibrosis. In one embodiment, the nephropathy is diabetic nephropathy.

In some embodiments of the treatment methods described herein, the inhibitor of TL-6 activity is an antibody or an antigen-binding fragment of an antibody. In some embodiments, the inhibitor is an IL-6 neutralizing antibody or an antigen-binding fragment thereof, such as, but not limited to, sirukumab, siltuximab, or olokizumab, or an antigen-binding fragment thereof. In some embodiments, the inhibitor is an antibody or an antigen-binding fragment which targets IL-6 receptor, such as, but not limited to, tocilizumab or sarilumab or an antigen-binding fragment thereof.

In some embodiments of the treatment methods described herein, the inhibitor of IL-6 activity provides trans-signaling blockade, such as, but not limited to, olamkicept (FE999301), or a fragment or variant thereof.

In some embodiments of the treatment methods described herein, the inhibitor of IL-6 activity is an inhibitor of intracellular signaling, such as, but not limited to, tofacitinib or CpG-stat3 siRNA.

In various embodiments of the treatment methods described herein, the method further comprises administering to the subject one or more additional agents effective to treat said condition or disease.

In some embodiments, the one or more additional agents comprise one or more cholesterol-lowering drugs, blood pressure-lowering therapies, antiinflammatory agents, anti-thrombotic agents, anti-coagulant agents, inhibitors of the renin-angiotensin aldosterone system (RAAS inhibitors), beta-adrenergic blockers, calcium channel blockers, blood sugar reducing medications, and/or Wnt inhibitors.

In some embodiments, the cholesterol-lowering drugs comprise statins, fibrates, and/or inhibitors of proprotein convertase subtilisin/kexin type 9.

In some embodiments, the blood pressure-lowering therapies comprise angiotensin-converting enzyme (ACE) inhibitors and/or angiotensin II receptor blockers (ARBs).

In some embodiments, the blood sugar reducing medications comprise metformin, insulin, glucose-dependent insulinotropic polypeptide (GIP), and glucagon-like peptide 1 (GLP-1), and/or sodium/glucose co-transporter 2 (SGLT2) inhibitors.

In some embodiments, the Wnt inhibitor comprise a compound having the structure according to formula (I):

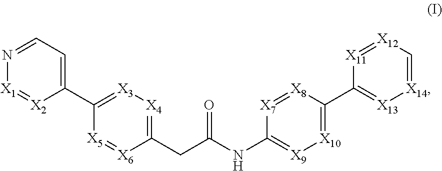

wherein X₁ and X₂ are selected from N and CR;
one of X₃, X₄, X₅ and X₆ is N and others are selected from N and CR;
one of X₇, X₈, X₉ and X₁₀ is N and others are selected from N and CR;
one of X₁₁, X₁₂, X₁₃ and X₁₄ is N and others are selected from N and CR, and
R is independently at each occurrence selected from hydrogen, halo, cyano, methyl, difluoromethyl, and trifluoromethyl,
   or a pharmaceutically acceptable salt thereof.

In various embodiments of the treatment methods described herein, the subject is human.

### BRIEF DESCRIPTION OF THE DRAWINGS

FIGS. 1A-1B show loss of endothelial glucocorticoid receptor (GR) results in a fibrogenic phenotype in the kidneys of diabetic mice. (FIG. 1A) Immunofluorescence analysis was performed in the kidneys of control and diabetic CD-1 and C57BL/6 mice using FITC-labeled GR, rhodamine-labeled CD31 and DAPI. Representative merged images are shown; original magnification 400×. Scale bar 50 μm. N=7 biologically independent mice/group combined from three separate experiments. Quantification of GR/CD-31 double-labelled cells is shown. Data are mean±SEM. (FIG. 1B) Western blot and qPCR analysis of GR protein and mRNA levels in isolated endothelial cells from the kidneys of control and diabetic CD-1 and C57BL/6 mice. Endothelial cells were isolated and cultured from six biologically independent mice/group. Three independent experiments were performed. Densitometry analysis of six samples/group are shown and are normalized to β-actin. mRNA expression from six independent cell culture samples was analyzed and normalized to 18S. Two independent experiments were performed in triplicate. Data are mean±SEM. For all panels, two-way ANOVA was used for analysis of statistical significance. Significance—*p<0.05, **p<0.01, ***p<0.001.

FIGS. 2A-2J demonstrate loss of endothelial GR worsens renal fibrosis in diabetic mice. (FIG. 2A) Schematic diagram, showing induction of diabetes in GR fl/fl; Tie1 Cre− (Control) GR fl/fl, Tie1 Cre+ (GR^{ECKO}) Apoe^{-/-}; GR fl/fl, Tie1 Cre− (Apoe^{-/-}) and Apoe^{-/-}; GR fl/fl, Tie1 Cre+ (DKO) mice. Five doses of STZ (50 mg/kg/day IP) were injected to induce diabetes and renal fibrosis. (FIGS. 2B-2I) Physiological parameters including body weight, blood glucose, kidney weight/body weight, heart weight/body weight, liver weight/body weight, albumin-to-creatinine ratio (ACR), plasma triglycerides and plasma cholesterol were measured. N=6 biologically independent mice/group from two separate experiments. (FIG. 2J). Masson trichome and Sirius red staining in kidneys of non-diabetic and diabetic control, GR^{ECKO} Apoe^{-/-} and DKO mice were analyzed. Representative images are shown; original magnification 300×. Relative area of fibrosis (%) and relative collagen (%) were measured using the ImageJ program. N=7 biologically independent mice/group from two separate experiments. Scale bar 50 μm. Data are mean±SEM. For all panels, two-way ANOVA was used for the analysis of statistical significance. Significance—*p<0.05, **p<0.01, ***p<0.001.

FIGS. 3A-3C show IL-6 neutralization rescues renal fibrosis in diabetes. (FIG. 3A) Plasma IL-6 levels in nondiabetic and diabetic mice were measured by cytokine array (Luminex). N=5 biologically independent mice/group. (FIG. 3B) Measurement of IL-6 in media of cultured endothelial cells isolated from the indicated groups. Endothelial cells were isolated and cultured from 6 biologically independent mice per group and were analyzed in triplicate. Data are mean±SEM. (FIG. 3C) IL-6 neutralization in diabetic GR^{ECKO} and diabetic DKO mice. Representative images are shown; original magnification 300×. Relative area of fibrosis (%) and relative collagen (%) were measured using the ImageJ program. N=7 biologically independent mice/group. Scale bar 50 μm. Data are mean±SEM. For all panels, one-way ANOVA with Tukey post-test was used for the analysis of statistical significance. *p<0.05, **p<0.01, ***p<0.001.

FIGS. 4A-4C demonstrates up regulation of Wnt signaling and fibrogenic markers with loss of endothelial GR. (FIG. 4A) Western blot analysis of vimentin, HIF1α, Snail1, active β-catenin, CPT1a and PPARα in isolated endothelial cells from the kidneys of nondiabetic and diabetic control and GR^{ECKO} mice. Endothelial cells were isolated and cultured from six biologically independent mice/group. Densitometry calculations from six samples/group combined from three independent experiments are shown and are normalized to β-actin or HSP90 as indicated. Representative blots are shown. Data are mean±SEM. (FIG. 4B) Western blot analysis of αSMA, fibronectin, active β-catenin and β-catenin in isolated endothelial cells from the kidneys of diabetic control, GR^{ECKO} Apoe^{-/-} and DKO mice. Endothelial cells were isolated and cultured from six biologically independent mice/group. Densitometry calculations from six samples/group combined from three independent experiments are shown and are normalized to β-actin or HSP90 as indicated. Data are mean±SEM. Representative blots are shown. (FIG. 4C) Immunofluorescence analysis of αSMA/CD31 and TGFβR1/CD31 was performed in the kidneys of nondiabetic and diabetic control, GR^{ECKO} Apoe^{-/-} and DKO mice. FITC-labeled αSMA, FITC-labeled TGFβR1 rhodamine-labeled CD31 and DAPI (nuclei) were used. Representative merged images are shown; original magnification 300×. Scale bar 50 μm. N=7 biologically independent mice/group. Data are mean SEM. For all panels, one-way ANOVA with Tukey post-test was used for analysis of statistical significance. Significance-*p<0.05, **p<0.01, ***p<0.001.

FIGS. 5A-5C shows Wnt inhibitor partially abrogates renal fibrosis in diabetic GRECKO and DKO mice. (FIG. 5A) Schematic diagram showing the treatment protocol of Wnt inhibition in diabetic control and GR^{ECKO} mice. (FIG. 5B) Physiological parameters including body weight, blood glucose and kidney weight/body weight were analyzed. N=6 biologically independent mice/group combined from two experiments. (FIG. 5C) Masson trichrome, Sirius red and PAS staining as well as immunohistochemical analysis of β-catenin levels in the kidneys of wnti-treated diabetic control and GR^{ECKO} mice. Representative images are shown; original magnification 300×. Relative area of fibrosis (%) and relative collagen deposition (%) were measured using the ImageJ program. N=8 biologically independent mice/group combined from three experiments were analyzed. Scale bar 50 μm. Data are mean SEM. For all panels one-way ANOVA with Tukey post-test was used for analysis of statistical significance. Significance—*p<0.05, **p<0.01, ***p<0.001.

FIGS. 6A-6E show metabolic reprogramming by loss of endothelial GR loss worsens diabetic kidney disease. (FIG. 6A) Radiolabeled ¹⁴C-palmitate uptake analysis in isolated endothelial cells from kidneys of nondiabetic and diabetic control, GR^{ECKO} Apoe^{-/-} and DKO mice. CPM of each sample were counted. Data were normalized to μg protein. Fold activity is presented. Endothelial cells isolated and cultured from six biologically independent mice/group were performed in triplicate and analyzed for ¹⁴C-palmitate uptake analysis. Data are mean±SEM. (FIG. 6B) Radiolabeled [¹⁴C]palmitate oxidation and [¹⁴CO₂]release were measured in isolated endothelial cells from kidneys of nondiabetic and diabetic control, GR^{ECKO} Apoe^{-/-} and DKO mice. CPM of each sample was counted. Data were normalized to μg protein. Fold activity is presented. Endothelial cells isolated and cultured from six biologically independent mice/group were performed in triplicate and analyzed for ¹⁴C-palmitate uptake analysis. Data are mean±SEM. (FIG. 6C) Body weight, blood glucose, and kidney weight/body weight were measured at the end of these experiments. N=6 biologically independent mice from two experiments. Ex vivo radiolabeled [¹⁴C]palmitate oxidation and [¹⁴CO₂]release were measured in kidneys. CPM of each sample was counted. Isolated endothelial cells from five biologically independent mice/group were analyzed in triplicate. Data are mean±SEM. (FIG. 6D) Masson trichrome, Sirius red and PAS staining were analyzed in the kidneys of control, etomoxir- and C75-treated diabetic mice. Representative images are shown; original magnification 300×. Relative area of fibrosis (%) and relative collagen deposition (%) were measured using the ImageJ program. N=8 biologically independent mice per/group from three independent experiments were analyzed. Scale bar 50 μm. Data are mean±SEM. (FIG. 6E) Immunohistochemical analysis of CPT1a and β-catenin. Representative images are shown; original magnification 300×. Scale bar 50 μm. N=6 biologically independent mice per group from three independent experiments. For all panels one-way ANOVA with Tukey post-test was used for analysis of statistical significance. Significance—*p<0.05, **p<0.01, ***p<0.001.

FIGS. 7A-7J show GR loss in endothelial cells reprograms central metabolism in renal tubular cells. (FIG. 7A) Conditioned media experimental design. HUVECs were transfected with scrambled or GR siRNA; after 6 h, the medium was changed and cells were incubated for 96 h. The subsequently harvested media was transferred to HK-2 cells. (FIG. 7B) Representative Western blotting analysis of E-cadherin, αSMA, TGFβR1 and active β-catenin expression. n=7 independent experiments. Representative blots from seven biologically independent samples/group were combined from four independent experiments. Densitometric analysis relative to β-actin is shown. (FIG. 7C) Immunofluorescence analysis of E-cadherin and α-SMA expression in conditioned medium treated TECs. For each slide, images of six different fields of view at Å˜400 magnification were evaluated. n=7 independent samples/group. Scale bar 30 μm. (FIG. 7D) [¹⁴C]palmitate oxidation measured by [¹⁴CO₂]release. CPM were counted and normalized to the protein in the well. n=8 independent samples/group. (FIG. 7E) Oxygen consumption rate (OCR) in conditioned medium treated-TECs; each data-point represents the mean of eight independent samples. Three independent experiments were performed. OCR was measured in a Seahorse XF96 analyzer. (FIG. 7F) Cellular ATP measurement. Eight independent samples were analyzed. (FIG. 7G) Relative mRNA levels determined by qRT-PCR of FAO regulators in conditioned media-treated TECs. n=7 independent samples per group performed in triplicate were analyzed. (FIG. 7H) Experimental design for conditioned media experiment. ECs from the kidneys of diabetic GR^{ECKO} and diabetic control mice were cultured for 96 h. The subsequently harvested media was transferred to TECs from diabetic control mice. n=7/group. (FIG. 7I) Representative Western blotting analysis of αSMA, TGFβR1 active β-catenin, E-cadherin, CPT1a and PPARα expression. Representative blots from seven biologically independent samples/group combined from three independent experiments are shown. Densitometry calculations are normalized to β-actin or HSP90 as indicated. (FIG. 7J) Relative mRNA levels determined by qRT-PCR of regulators of FAO in conditioned media-treated isolated TECs. n=6 independent replicates per group. Data are mean±SEM. Student's t-test (unpaired two-tailed) was used for FIGS. 7B-7F; FIG. 7I; for FIG. 7G and FIG. 7J, one-way Anova with Tukey post hoc test was used for analysis of statistical significance. Significance-*p<0.05, **p<0.01, ***p<0.001.

FIG. 8 shows a graphical representation of the role of GR in endothelial cell homeostasis. GR is crucial for endothelial cell homeostasis and its loss causes homeostatic disruption through release of the pro-inflammatory cytokine IL-6 and aberrant activation of Wnt signaling which lead to defective fatty acid oxidation and associated mesenchymal activation.

FIGS. 9A-9C show effect of adrenalectomy on renal fibrosis. Adrenalectomized and non-adrenalectomized control and diabetic mice of CD-1 and C57BL/6 strains were evaluated 4 weeks after bilateral adrenal surgery. (FIG. 9A) Measurement of plasma corticosterone in control and diabetic CD-1 and C57BL/6 mice. n=7 biologically independent mice/group combined from two separate experiments. (FIG. 9B) Blood glucose measurement in adrenalectomized and non-adrenalectomized mice. n=7 biologically independent mice/group combined from two separate experiments. (FIG. 9C) Masson trichrome and Sirius red staining in the kidneys of adrenalectomized and non-adrenalectomized control and diabetic mice. Representative images are shown; original magnification 300×. Area of fibrosis (%) and relative collagen deposition (%) were measured using the ImageJ program. n=7 biologically independent mice/group combined from two separate experiments. Scale bar 50 μm. Data are mean±SEM. One-way Anova with Tukey multiple comparison post hoc test was used to calculate statistical significance. Significance—*p<0.05, **p<0.01, ***p<0.001. C-CD1: nondiabetic control CD1, DM-CD1: diabetic CD-1, C-BL6: nondiabetic control C57BL/6, DM-BL6: diabetic C57BL/6 mice and ADX: adrenalectomized.

FIG. 10 shows determination of kidney endothelial cell purity. Top: Flow cytometry cell purity analysis in isolated endothelial cells from kidneys. CD31+CD45− cells are shown. Bottom: The gating strategy in unstained, blocked endothelial cells is shown.

FIGS. 11A-11B show analysis of GR protein and mRNA levels in isolated endothelial cells. (FIG. 11A) Western blot analysis of GR protein level and (FIG. 11B) qPCR analysis of GR mRNA expression were analyzed in isolated endothelial cells from the kidneys of control and GR^{ECKO} mice. n=7 biologically independent control mice, n=8 biologically independent GR^{ECKO} mice. Three independent experiments were analyzed. Data are mean SEM. Student t-test (unpaired two-tailed) was used for analysis of statistical significance. Significance—*p<0.05, **p<0.01, ***p<0.001.

FIG. 12 shows physiological characteristics of nondiabetic mice. Body weight, blood glucose, kidney/body weight, heart/body weight, liver/body weight, albumin-to-creatinine ratio, plasma triglycerides, plasma cholesterol and systolic blood pressure in nondiabetic control, GR^{ECKO}, Apoe^{-/-} and DKO mice are shown. n=6 biologically independent mice/group, except for blood pressure where n=8 biologically independent mice/group, are combined from two separate experiments. Data are mean±SEM. One-way Anova with Tukey multiple comparison post-test was used to calculate statistical significance. Significance—*p<0.05, **p<0.01, ***p<0.001.

FIGS. 13A-13B show evaluation of glomerular fibrosis in nondiabetic and diabetic mice. (FIG. 13A) Periodic acid Schiff (PAS) staining in the kidneys of nondiabetic and diabetic control, GR^{ECKO}, Apoe−/− and DKO mice. Scale bar 50 μm. n=7 biologically independent mice/group combined from two separate experiments. Representative images are shown. (FIG. 13B) Immunohistochemical analysis of vimentin expression in the kidneys of nondiabetic and diabetic control, GR^{ECKO}, Apoe^{-/-} and DKO mice. n=7 biologically independent mice/group combined from two separate experiments. Scale bar 50 μm. Representative images are shown; original magnification 200×.

FIG. 14 shows expression of Snail1, PPARα and HIF1α in nondiabetic and diabetic mice. Immunohistochemical analysis of Snail1, PPARα and HIF1α expression in the kidneys of nondiabetic and diabetic control, GR^{ECKO}, Apoe^{-/-} and DKO mice. Scale bar 50 μm. n=7 biologically independent mice/group combined from two separate experiments. Representative images are shown; original magnification 200×.

FIG. 15 shows collagen I and fibronectin deposition in nondiabetic and diabetic mice. Immunofluorescence analysis of collagen I and fibronectin was performed in the kidneys of non-diabetic and diabetic control, GR^{ECKO}, Apoe^{-/-} and DKO mice. FITC labeled-collagen I, rhodamine labeled-fibronectin and DAPI were used. Scale bar 50 μm. n=7 biologically independent mice/group. Representative images from two separate experiments are shown; original magnification 300×.

FIGS. 16A-16C shows loss of endothelial cell GR worsens fibrosis in a mouse model of unilateral ureteral obstruction (UUO). (FIG. 16A) Schematic presentation of UUO model. Left kidneys were ligated in control littermates and GR^{ECKO} mice. On days 5 and 10 kidneys were excised. (FIG. 16B) Masson trichrome and Sirius red staining in contralateral and UUO-operated kidneys were analyzed. Representative images are shown; original magnification 200×. Area of fibrosis (%) and relative collagen deposition (RCD %) were measured using the ImageJ program. n=8 biologically independent mice/group combined from three independent experiments. Scale bar 50 μm. Data are mean±SEM. Two-way Anova with Tukey multiple comparison post hoc test was used to calculate statistical significance. Significance—*p<0.05. (FIG. 16C) Immunofluorescence analysis of collagen I, aquaporin/α-SMA and aquaporin/fibronectin were performed in the contralateral and UUO-operated kidneys of control and GR^{ECKO} mice. FITC labeled-collagen I, α-SMA and fibronectin, rhodamine-labeled aquaporin and DAPI were used. Representative images are shown; original magnification 300×. Scale bar 50 μm. n=7 biologically independent mice/group combined from two experiments. Con: control; Contra: contralateral kidneys; FN: fibronectin.

FIGS. 17A-17B show dexamethasone treatment in urinary obstruction. (FIG. 17A) Masson trichrome staining in dexamethasone-treated and vehicle-treated UUO mice; original magnification 200×. Mice were treated with dexamethasone 1 mg/kg in the drinking water until the day of sacrifice. Time dependent analysis on days 10 and 15 are presented. Representative images are shown. Relative area of fibrosis (%) was measured using the ImageJ program. n=7 biologically independent mice/group combined from two independent experiments. Scale bar 50 μm. Data are mean±SEM. One-way Anova with Tukey multiple comparison post hoc test was used to calculate statistical significance. Significance—***p<0.001. (FIG. 17B) Masson trichrome staining (original magnification 200×) in dexamethasone-treated and vehicle-treated non-diabetic CD-1 mice. Representative images are shown. Relative area of fibrosis (%) was measured using the ImageJ program. n=6 biologically independent mice/group. Scale bar 50 μm. Data are mean SEM. Data were analyzed by Student's-test (unpaired, two-tailed).

FIG. 18 shows diabetic kidney disease is associated with cytokine and chemokine reprogramming. Plasma cytokines and chemokines were measured in nondiabetic control and diabetic CD-1 and C57BL/6 mice by using a cytokine array analysis (Luminex). n=5 biologically independent mice/group. Data are mean±SEM. One-way Anova with Tukey multiple comparison post hoc test was used to calculate statistical significance. Significance—*p<0.05, **p<0.01, ***p<0.001. C-CD1: nondiabetic control CD1, DM-CD1: diabetic CD-1, C-BL6: nondiabetic control C57BL/6, DM-BL6: diabetic C57BL/6 mice.

FIG. 19A-19B show Cytokine and chemokine reprogramming in non-diabetic and diabetic GR^{ECKO} and DKO mice. (FIG. 19A) Plasma cytokines and chemokines were measured in non-diabetic and diabetic GR^{ECKO} and DKO mice by using a cytokine array analysis (Luminex). n=5 biologically independent mice/group. Data are mean±SEM. (FIG. 19B) Relative gene expression analysis of the cytokines shown were evaluated in diabetic kidneys. n=6 biologically independent mice/group. 18S was used to normalize the expression level. Data are mean±SEM. In both panels, one-way Anova with Tukey multiple comparison post hoc test was used to calculate statistical significance. Significance—*p<0.05, **p<0.01, ***p<0.001.

FIGS. 20A-20D show IL-6 neutralization rescues the fibrotic phenotype in kidneys of diabetic CD-1 mice. IL-6 IgG and control IgG were injected intraperitoneally three times per week for four weeks at a dose of 3 mg/kg in both non-diabetic and diabetic CD-1 mice. (FIG. 20A-20B) Western blot analysis of vimentin and α-SMA in recombinant IL1β-, IL-6-, TNFα- and TGFβ-stimulated isolated endothelial cells from CD-1 mice. Representative blots from three independent experiments were analyzed. Densitometry calculations are shown relative to -actin. (FIG. 20C) Western blot analysis of vimentin in TGF β-stimulated and unstimulated endothelial cells treated with N-IL1β (neutralization antibody—IL-1β), and N-IL-6 (neutralization antibody—IL-6) antibodies. Representative blots from three independent experiments were evaluated. Densitometry calculations are shown relative to β-actin. (FIG. 20D) IL-6 neutralization in control and diabetic CD-1 mice. Representative images of Masson trichrome and Sirius red staining (original magnification 300×) are shown. n=7 biologically independent mice/group combined from two independent experiments. Relative area of fibrosis (%) and relative collagen (%) were measured using the ImageJ program. Scale bar 50 μm. Data are mean±SEM. In all panels, one-way Anova with Tukey multiple comparison post-test was used to calculate statistical significance. Significance—*p<0.05, **p<0.01, ***p<0.001.

FIG. 21 shows IL-6 neutralization in nondiabetic GR^{ECKO} and DKO mice. IL-6 IgG and control IgG were injected intraperitoneally three times per week for four weeks at a dose of 3 mg/kg in both non-diabetic GR^{ECKO} and DKO mice. Representative images (original magnification 300×) of Masson trichrome (top row) and Sirius red (bottom row) staining are shown. n=6 biologically independent mice/group combined from two experiments. Relative area of fibrosis (%) and relative collagen (%) were measured using the ImageJ program. Scale bar 50 μm. Data are mean±SEM. One way ANOVA with Tukey post-test was used for analysis of statistical significance.

FIGS. 22A-22C shows gene expression analysis of Wnt signaling and fibrogenic markers. (FIG. 22A) Relative mRNA levels determined by qRT-PCR of Axin2, Tcf, α-SMA, fibronectin, Snail1, Hif1α and Ppara were analyzed in isolated endothelial cells from the kidneys of nondiabetic and diabetic control, GR^{ECKO}, Apoe^{-/-} and DKO mice. Endothelial cells from n=6 biologically independent mice/group were evaluated. The combined data from four independent experiments were analyzed. Data are mean±SEM. (FIG. 22B) Relative mRNA levels determined by qRT-PCR of Axin2, Tcf, and fibronectin were analyzed in isolated endothelial cells from cultured glomeruli from kidneys of nondiabetic and diabetic control and GR^{ECKO} mice. n=6 biologically independent mice/group were analyzed. The combined data from three independent experiments were analyzed. Data are mean SEM. In (FIG. 22A) and (FIG. 22B) one-way ANOVA with Tukey post-test was used for analysis of statistical significance. Significance—*p<0.05, **p<0.01, ***p<0.001. (FIG. 22C) Immunohistochemical analysis of -catenin in the kidneys of indicated groups. Representative images are shown; original magnification 300×. n=7 biologically independent mice/group combined from three independent experiments. Scale bar 50 μm.

FIGS. 23A-23C show endothelial cell characteristics. (FIG. 23A) Endothelial cell leakage in the kidneys of the nondiabetic and diabetic, control, GR^{ECKO}, Apoe^{-/-} and DKO mice was assessed 2 hours after fluorescein isothiocyanate (FITC)-dextran injection in mice. FITC-dextran, rhodamine-labeled CD31 and DAPI were used. Merged images and representative pictures (original magnification 300×) are shown. Arrows indicate FITC-dextran only-labeled cells and areas of leakage. n=7 biologically independent mice/group combined from three experiments. Scale bar 50 μm. Quantification is shown below the images. ND: non-diabetic, D: diabetic. Data are mean±SEM. (FIG. 23B) The permeability of isolated endothelial cell monolayers was assessed using Transwell inserts by measuring the passage of FITC-dextran added in the top compartment to the bottom compartment. After 24 hrs, the concentration of FITC-dextran in the bottom compartment was measured with a fluorescence plate reader. Endothelial cells isolated from six biologically independent mice were evaluated. Three independent experiments were analyzed. (FIG. 23C) Endothelial cell density (%) as measured by CD31 staining from FIG. 23A. Endothelial cells isolated from six biologically independent mice were evaluated. Three independent experiment were analyzed. Data are mean±SEM. In all panels, one-way Anova with Tukey multiple comparison post hoc test was used to calculate statistical significance. Significance—*p<0.05, **p<0.01, ***p<0.001.

FIG. 24A-24J show inhibition of Wnt signaling abolished the fibrogenic phenotype in mice. (FIG. 24A) Immunohistochemical analysis of -catenin expression in the kidneys of control and diabetic CD-1 and C57BL/6 mice. n=8 biologically independent mice/group combined from two independent experiments were evaluated. Scale bar 50 μm. Representative images are shown; original magnification 200×. (FIG. 24B) Relative mRNA expression determined by qRT-PCR of Axin2, Tcf and fibronectin, in kidneys of control and diabetic CD-1 and C57BL/6 mice. n=6 biologically independent mice/group combined from two experiments were evaluated. Expression was normalized to the internal control 18S. (FIG. 24C) Schematic diagram representing the treatment protocol of Wnt inhibitor (LGK974; 5 mg/kg body weight) in diabetic CD-1 mice and (FIG. 24D) in UUO mice. (FIG. 24E) Masson trichrome and Sirius red staining in the kidneys of diabetic and Wnt inhibitor-treated diabetic CD-1 mice. Representative images (original magnification 300×) are shown. Relative area of fibrosis (%) and relative collagen deposition (%) were measured using the ImageJ program. n=8 biologically independent mice/group combined from two experiments were evaluated. Scale bar 50 μm. (FIG. 24F) Masson trichrome and Sirius red staining in the kidneys of UUO and Wnt inhibitor-treated UUO mice. Representative images (original magnification 300×) are shown. Relative area of fibrosis (%) and relative collagen deposition (%) were measured using the ImageJ program. n=8 biologically independent mice/group combined from two experiments were evaluated. Scale bar 50 μm. (FIGS. 24G-24H) GR protein levels in CD31-positive cells were analyzed by immunofluorescence in the kidneys of Wnti-treated diabetic mice and Wnti-treated UUO mice. FITC-labeled GR, rhodamine-labeled CD31 and DAPI were used. Merged images and representative pictures are shown; original magnification 300×. n=7 biologically independent mice/group combined from four experiments were evaluated. Scale bar 50 μm. (FIGS. 24I-24J) Immunohistochemical analysis of active -catenin expression in Wnti-treated diabetic mice and Wnti-treated UUO mice. n=7 biologically independent mice/group combined from three experiments were evaluated. Scale bar 30 μm. Data are mean SEM. In all panels, one-way Anova with Tukey multiple comparison post hoc test was used to calculate statistical significance. Significance—*p<0.05, **p<0.01, ***p<0.001.

FIG. 25 shows inhibition of Wnt signaling disrupts cytokine and chemokine reprogramming in diabetic mice. Plasma cytokines and chemokines were measured in diabetic CD-1 mice with and without Wnti treatment by using a cytokine array analysis (Luminex). n=5 biologically independent mice/group. Data are mean±SEM. Student's-test (unpaired two-tailed) was used for analysis of statistical significance. Significance—*p<0.05.

FIG. 26 shows physiological characteristics of Wnt inhibitor treatment in nondiabetic control and GR^{ECKO} mice. Physiological parameters including body weight, blood glucose, and kidney weight/body weight are shown. n=7 biologically independent mice/group combined from two experiments were analyzed. Data are mean±SEM. One-way Anova with Tukey multiple comparison post hoc test was used to calculate statistical significance.

FIGS. 27A-27B Histological analysis of kidneys from Wnt inhibitor-treated nondiabetic control and GR^{ECKO} mice. (FIG. 27A) Masson trichrome staining (MITS) and Sirius red staining in Wnt inhibitor-treated nondiabetic control and GR^{ECKO} mice. Representative images are shown; original magnification 200×. Area of fibrosis (%) and relative collagen deposition (%) were measured using the ImageJ program. N=5 biologically independent mice/group were analyzed. Data are mean±SEM. Scale bar 50 μm. (FIG. 27B) Immunohistochemical analysis of -catenin expression in the kidneys of Wnt inhibitor-treated nondiabetic control and GR^{ECKO} mice. Scale bar 30 μm. n=6 biologically independent mice/group combined from two independent experiments were analyzed. Representative images are shown; original magnification 300×. One-way Anova with Tukey multiple comparison post hoc test was used to calculate statistical significance. Significance—*p<0.05, **p<0.01, ***p<0.001.

FIG. 28 shows expression of Snail1, PPARα and HIF1α in Wnt inhibitor-treated nondiabetic and diabetic mice. Immunohistochemical analysis of Snail1, PPAR. and HIF1. expression in the Wnti-treated and untreated control, and GR^{ECKO} mice. Scale bar 50 μm. n=7 biologically independent mice/group combined from three independent experiments were analyzed. Representative images are shown; original magnification 200×.

FIG. 29 shows Snail1, PPARα, active-β-catenin and HIF1α in isolated cells from Wnt inhibitor-treated diabetic mice. Western blot analysis of Snail1, PPARα, active-β-catenin, and HIF1α proteins in isolated endothelial cells. Representative blots are shown. Densitometry calculations are combined from three independent experiments Data are mean±SEM. One-way Anova with Tukey multiple comparison post hoc test was used to calculate statistical significance. Significance—*p<0.05, **p<0.01, ***p<0.001.

FIG. 30 shows EndMT and EMT analysis in kidneys from Wnt inhibitor-treated mice. Immunofluorescence analysis of α-SMA/CD31/DAPI and E-cadherin/α-SMA/DAPI in kidneys of Wnt inhibitor-treated nondiabetic and diabetic mice. Top panel: α-SMA FITC labelled, CD31 rhodamine labelled and DAPI blue; Lower panel: E-Cadherin FITC-green labelled, α-SMA red rhodamine labelled and DAPI blue. Representative images are shown; original magnification 400×. Scale bar 50 μm. n=6 biologically independent mice/group combined from three independent experiments were analyzed. NC: non-diabetic control; DC: diabetic control; D: diabetic.

FIG. 31A-31B show Fatty acid uptake and fatty acid oxidation in isolated endothelial cells. (FIG. 31A) Fatty acid uptake was measured as [¹⁴C]palmitate uptake. (FIG. 31B) Fatty acid oxidation, as represented by [¹⁴CO₂]released. [¹⁴C]palmitate was used as substrate and CPM counts were normalized to protein. In both cases, endothelial cells isolated from n=5 biologically independent mice/group were analyzed in triplicate. Data are mean±SEM. One-way Anova with Tukey multiple comparison post hoc test was used to calculate statistical significance. Significance—*p<0.05, **p<0.01, ***p<0.001.

FIG. 32 shows gene expression of fatty acid transporter proteins in nondiabetic and diabetic mice Relative mRNA levels determined by qRT-PCR of Fatp1, Fatp4, and Cd36 were analyzed in isolated endothelial cells from kidneys of nondiabetic and diabetic control and GR^{ECKO} mice. Endothelial cells isolated from n=5 biologically independent mice/group were analyzed in triplicate. Data are mean±SEM. One-way ANOVA with Tukey post-test was used for the analysis of statistical significance. Significance—*p<0.05.

FIG. 33A-33F show endothelial GR is essential for the action of anti-dyslipidemic drugs in the diabetic kidney disease. (FIG. 33A) Masson trichrome (MTS) and Sirius red staining in kidneys of control, diabetic, fenofibrate-, etomoxir-, C75- and simvastatin-treated diabetic mice. Representative images (original magnification 300×) are shown. Relative area of fibrosis (%) and relative collagen deposition (%) were measured using the ImageJ program. n=6 biologically independent mice/group combined from two experiments were analyzed. Scale bar 50 μm. (FIG. 33B) Co-immunolabeling of GR/CD31 was analyzed by fluorescence microscopy. FITC-labeled GR, rhodamine-labeled CD31 and DAPI were used. Representative images are shown; original magnification 300×. Scale bar 50 μm. n=6 biologically independent mice/group combined from two experiments were analyzed. (FIG. 33C) PCR gene analiof fibronectin and α-SMA in the kidneys. 18S was used as an internal control. n=6 biologically independent mice/group. (FIG. 33D) Blood glucose measurements in control, diabetic, fenofibrate-, etomoxir-, C75- and simvastatin-treated diabetic mice. n=6 biologically independent mice/group. (FIG. 33E) Radiolabeled [¹⁴C]palmitate oxidation and [¹⁴CO₂]release were measured. CPM of each sample was counted. Endothelial cells isolated from six biologically independent mice were evaluated. Three independent experiments were analyzed. Data are mean±SEM (FIG. 33F) Immunohistochemical analysis of CPT1a and β-catenin was analyzed in the kidneys of control, diabetic, fenofibrate-, C75-, etomoxir- and simvastatin-treated diabetic mice. n=6 biologically independent mice/group. Representative images are shown; original magnification 400×. Scale bar 50 μm. Data are mean SEM. In all panels, one-way Anova with Tukey multiple comparison post hoc test was used to calculate statistical significance. Significance—*p<0.05, **p<0.01, ***p<0.001. Con: control, DM: diabetic, feno: fenofibrate.

FIG. 34 shows physiologic characteristics of nondiabetic etomoxir- and C75-treated mice. Body weight, blood glucose, and kidney/body weight ratio and fatty acid oxidation as represented by [¹⁴CO₂]release are shown. [¹⁴C]palmitate was used as substrate and CPM counts were normalized by protein. n=6 biologically independent mice/group. Data are mean±SEM. One-way Anova with Tukey multiple comparison post hoc test was used to calculate statistical significance. Significance—*p<0.05.

FIG. 35 shows histologic analysis of kidneys from non-diabetic etomoxir- and C75-treated mice. Masson trichrome staining (upper panel) and Sirius red (lower panels) in etomoxir- and C75-treated non-diabetic control and GR^{ECKO} mice. Etomoxir (20 mg/kg) and C75 (15 mg/kg) were dosed (IP) three times per week for 3 weeks in GR^{ECKO} and control littermates. Representative images are shown; original magnification 400×. Area of fibrosis (%) and relative collage deposition (%) in n=6 biologically independent mice/group were combined from two independent experiments. Scale bar 50 μm. Data are mean±SEM. One-way Anova with Tukey multiple comparison post hoc test was used to calculate statistical significance. Significance-**p<0.01.

FIG. 36 shows GR overexpression suppresses fibrogenic markers in vitro. Western blotting and corresponding quantification of the protein level of GR and collagen I (normalized to β-actin) and β-catenin and α-SMA (normalized to HSP90) in HUVECs subjected to either empty vector, GR overexpression vector, empty vector+TGF β2 or GR overexpression vector+TGF β2. Representative blots are shown. Data combined from three independent experiments were analyzed. Data are mean±SEM. One-way Anova with Tukey multiple comparison post hoc test was used to calculate statistical significance. Significance—***p<0.001.

FIG. 37 shows EC-specific inhibition of canonical Wnt produces significant improvement in renal and heart fibrosis and provides support that the Wnt canonical pathway is involved. An endothelial-specific LRP5/6 KO mouse model, a novel mouse model which inhibits canonical Wnt signaling in the LRP5/6 receptor, was used in the generation of these data.

FIGS. 38A-38H show pharmacological inhibition of Wnt signaling abolishes fibrogenic phenotype in db/db mice. (FIG. 38A) Postprandial blood glucose measurements in the Wnt inhibitor treatments in db/db mice and untreated control db/db mice. LGK-974 (Wnt inhibitor) was treated at 5 mg/kg body weight dose orally by gavage. (FIG. 38B) GTT analysis in the Wnt inhibitor treatments in db/db mice and untreated control db/db mice. N=6 were analyzed in each group. (FIG. 38C) Area under curve (AUC) in the Wnt inhibitor treatments in db/db mice and untreated control db/db mice. N=6 were analyzed in each group. (FIG. 38D) Physiological parameters such as body weight, epididymal fat, kidney weight, liver weight and heart weight in the Wnt inhibitor treatments in db/db mice and untreated control db/db mice. N=6 were analyzed in each group. (FIG. 38E) MTS and Sirius red staining analysis in the Wnt inhibitor treatments in db/db mice and untreated control db/db mice. Representative pictures of 30× are shown. N=6 were analyzed in each group. Relative area fibrosis (RAF) and relative collagen deposition (RCD) were quantified using Image J software program. (FIG. 38F) MTS and Sirius red staining analysis in the heart sections in the Wnt inhibitor treatments in db/db mice and untreated control db/db mice. Representative pictures of 20× are shown. N=6 were analyzed in each group. (FIG. 38G) MTS in the liver sections (FIG. 38H) MTS in the adipose tissue sections in the Wnt inhibitor treatments in db/db mice and untreated control db/db mice. Representative pictures of 20× are shown. N=6 were analyzed in each group. Scale bar: 50 μm. All data represent the mean±SEM. Unpaired t-test was employed for statistical significance. *P≤0.05.

### DETAILED DESCRIPTION

Unless defined otherwise, technical and scientific terms used herein have the same meaning as commonly understood by one of ordinary skill in the art to which this disclosure belongs.

As used herein, the singular forms “a”, “an” and “the” include plural references unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

The term “antibody” refers to all isotypes of immunoglobulins (e.g., IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies. Immunoglobulin molecules can be of any class (e.g., IgG1, IgG2, IgG3, IgG4, IgM1, IgM2, IgA1 and IgA2) or subclass.

The term “antigen-binding fragment” refers to any proteinaceous structure that may exhibit binding affinity for a particular antigen. Antigen-binding fragments include those produced by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more complementarity determining regions (CDRs) of an antibody known to bind a particular antigen. Examples of suitable antigen-binding fragments include, but not limited to, single-chain molecules such as Fab, F(ab′)2, Fc, Fabc, Fv molecules, scFv, and disulfide-linked Fvs (sdFv), intrabodies, diabodies, minibodies, linear antibodies, single domain antibodies such as sdAb (either VL or VH), camelid nanobodies (VHH domains), multi-specific antibodies formed from antibody fragments, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782 (which is incorporated herein by reference in its entirety), bivalent fragments comprising two Fab fragments linked by a disulfide bridge at the hinge region, a Fd fragment consisting essentially of the VH and CH1 domains, a dAb fragment, or an isolated CDR, and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. The phrase “an antibody or antigen-binding fragment thereof” may be used to denote that a given antigen-binding fragment incorporates one or more amino acid segments of the antibody referred to in the phrase.

The term “epitope” refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of nonlinear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The terms “patient”, “individual”, “subject”, “mammal”, and “animal” are used interchangeably herein and refer to mammals, including, without limitation, human, veterinary animals (e.g., cats, dogs, cows, horses, sheep, pigs, etc.) and experimental animal models (e.g., mouse, rabbit, rat). Animals include all vertebrates, e.g., mammals and non-mammals, such as mice, sheep, dogs, cows, avian species, ducks, geese, pigs, chickens, amphibians, and reptiles. In a preferred embodiment, the subject is a human. In some embodiments, a subject is in need of prevention or treatment for dyslipidemia or a related disorder or condition.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like.

The term “pharmaceutically acceptable”, as used herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” or “a pharmaceutically acceptable carrier” as used herein, refers to any clinically useful solvents, diluents, adjuvants, excipients, recipients, vehicles and the like for use in preparing admixtures of a pharmaceutical composition.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to 10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

In accordance with the present disclosure, there may be employed conventional molecular biology, microbiology, and recombinant DNA techniques within the skill of the art. See, e.g., Sambrook, Fritsch and Maniatis, Molecular Cloning: A Laboratory Manual, 2nd ed., Cold Spring Harbor Laboratory Press, Cold Spring Harbor, New York, 1989 (herein “Sambrook et al., 1989”); DNA Cloning: A Practical Approach, Volumes I and II (Glover ed. 1985); Oligonucleotide Synthesis (Gait ed. 1984); Nucleic Acid Hybridization (Hames and Higgins eds. 1985); Transcription And Translation (Hames and Higgins eds. 1984); Animal Cell Culture (Freshney ed. 1986); Immobilized Cells And Enzymes (IRL Press, 1986); B. Perbal, A Practical Guide To Molecular Cloning (1984); Ausubel et al. eds., Current Protocols in Molecular Biology, John Wiley and Sons, Inc. 1994; among others.

In one aspect of the present disclosure, provided is a method for treating a condition or a disease in a subject in need thereof, which method comprises administering to the subject a therapeutically effective amount of an inhibitor of IL-6 activity.

The condition or disease that can be treated by the methods of the present disclosure include, but are not limited to, organ fibrosis, nephropathy, dyslipidemia, hypertension, hyperlipidemia, hypercholesterolemia, cardiovascular disease, peripheral artery disease, atherosclerosis, coronary artery disease, coronary heart disease, and stroke.

In some embodiments, the organ fibrosis is renal fibrosis.

In some embodiments, the nephropathy is diabetic nephropathy.

In some embodiments, the method is effective to prevent or treat a nephropathy. In some embodiments, the method is effective to reduce fibrosis in the kidney of the subject. In some embodiments, the method is effective to reduce collagen deposition in the kidney of the subject. In some embodiments, the method is effective to reduce accumulation of collagen in the kidney of the subject. In some embodiments, the method is effective to reduce glomerulosclerosis in the subject.

The term “dyslipidemia” refers to abnormal levels of lipoproteins in blood plasma including both depressed and/or elevated levels of lipoproteins (e.g., elevated levels of LDL and/or VLDL, and depressed levels of HDL).

The term “hypercholesterolemia,” as used herein, refers to a condition in which cholesterol levels are elevated above a desired level. In some embodiments, this denotes that serum cholesterol levels are elevated. In some embodiments, the desired level takes into account various “risk factors” that are known to one of skill in the art (and are described or referenced herein). “Familial hypercholesterolemia” refers hypercholesterolemia caused by a mutation in a gene located on chromosome 19.

The term “nephropathy” as used herein refers to a disease, dysfunction or a non-function of one or both kidneys. The term “diabetic nephropathy” as used herein includes both incipient and overt stages of diabetic nephropathy, whether diagnosed or not, though diabetic nephropathy is most typically as diagnosed by a clinician or physician.

As used herein, the term “atherosclerosis” refers to a disease of the arteries characterized by the narrowing of arteries due to plaque buildup in the arteries. The term “atherosclerosis-related disorder” refers to atherosclerotic cardiovascular disease (ASCVD) and other such cholesterol deposition-driven chronic inflammatory diseases. Atherosclerosis-related disorders include, without limitation: ASCVD, coronary heart disease, such as myocardial infarction, angina, and coronary artery stenosis; cerebrovascular disease, such as transient ischemic attack, ischemic stroke, and carotid artery stenosis; peripheral artery disease, such as claudication; aortic atherosclerotic disease, such as abdominal aortic aneurysm and descending thoracic aneurysm; hypertension; peripheral vascular disease; coronary artery disease; aortic aneurysm; carotid artery disease; coronary atherosclerosis; heart attack; acute coronary syndromes, and stroke.

As used herein, the term “coronary heart disease” refers to a narrowing of the small blood vessels that supply blood and oxygen to the heart, which is often a result of atherosclerosis.

In various embodiments of the methods described herein, the subject is human.

In some embodiments, the inhibitor of IL-6 activity is an antibody or antigen-binding fragment.

In some embodiments, the inhibitor is an IL-6 neutralizing antibody or an antigen-binding fragment thereof. In some embodiments, the inhibitor is sirukumab, siltuximab, or olokizumab, or an antigen-binding fragment thereof.

In some embodiments, the inhibitor is an antibody or an antigen-binding fragment thereof which targets IL-6 receptor. In some embodiments, the inhibitor is tocilizumab or sarilumab, or an antigen-binding fragment thereof.

In some embodiments, an antibody or antigen-binding fragment described herein specifically binds to IL-6 or IL-6 receptor with high affinity, for example, a K_{D} of less than about 1×10⁻⁸ M, such as but not limited to, about 1-9.9 (or any range or value therein, such as 1, 2, 3, 4, 5, 6, 7, 8, or 9)×10⁻⁹ M, 10⁻¹⁰ M, 10⁻¹¹ M, 10⁻¹² M, 10⁻¹³ M, 10⁻¹⁴ M, 10⁻¹⁵ M or any range or value therein, as determined by, for example, bio-layer interferometry assay, surface plasmon resonance, or the Kinexa method, as practiced by those of skill in the art. One example affinity is equal to or less than 1×10⁻¹⁰ M. Another example affinity is equal to about 3.8×10⁻¹¹ M.

Methods of testing antibodies for the ability to bind to the target peptide or any portion thereof are known in the art and include any antibody-antigen binding assay, such as, for example, bio-layer interferometry assay, radioimmunoassay (RIA), Western blot, enzyme-linked immunosorbent assay (ELISA), immunoprecipitation, and competitive inhibition assays. In some embodiments, K_{D} values described herein are determined using a bio-layer interferometry assay.

In some embodiments, the antibody or antigen-binding fragment described herein is a human antibody, a monoclonal antibody, a humanized antibody, a single chain antibody, a Fab, a Fab′, a F(ab′)2, a Fv, or a scFv.

Antibodies or antigen-binding fragments thereof that compete for binding to IL-6 or IL-6 receptor with the antibody or antigen-binding fragment described herein may also be used in the methods of the present disclosure. The term “competes” or “cross-competes”, as used herein, means an antibody or antigen-binding fragment thereof binds to an antigen and inhibits or blocks the binding of another antibody or antigen-binding fragment thereof. The term also includes competition between two antibodies in both orientations (wherein a first antibody that binds and blocks binding of the second antibody and vice-versa). In some embodiments, a competing antibody and an antibody described herein may bind to the same epitope. Alternatively, a competing antibody and an antibody described herein may bind to different, but overlapping epitopes such that binding of one inhibits or blocks the binding of the second antibody, e.g., via steric hindrance. Cross-competition between antibodies may be measured by methods known in the art, for example, by a real-time, label-free bio-layer interferometry assay. Cross-competition between two antibodies may be expressed as the binding of the second antibody that is less than the background signal due to self-self binding (wherein first and second antibodies is the same antibody). Cross-competition between two antibodies may be expressed, for example, as % binding of the second antibody that is less than the baseline self-self background binding (wherein first and second antibodies is the same antibody).

Antibodies or antigen-binding fragments thereof that bind to the same epitope as the antibody or antigen-binding fragment described herein may also be used in the methods of the present disclosure.

The antibodies or antigen-binding fragments described herein may of any one of various antibody isotypes, such as IgM, IgD, IgG, IgA and IgE. In some embodiments, the antibody isotype is IgG1, IgG2, IgG3, or IgG4 isotype. In some embodiments, the antibody isotype is IgA1 or IgA2. Antibody or antigen-binding fragment thereof specificity is largely determined by the amino acid sequence, and arrangement, of the CDRs. Therefore, the CDRs of one isotype may be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

In some embodiments, the inhibitor of IL-6 activity provides trans-signaling blockade. In one embodiment, the inhibitor is olamkicept (FE999301), or a fragment or variant thereof.

In some embodiments, the inhibitor of IL-6 activity is an inhibitor of intracellular signaling. In some embodiments, the inhibitor is tofacitinib or CpG-stat3 siRNA.

Polypeptide compounds (such as antibodies or antigen-binding fragments) described herein include variants having single or multiple amino acid substitutions, deletions, or additions that retain the biological properties (e.g., binding affinity or immune effector activity) of the described polypeptide compounds.

These variants may include: (i) variants in which one or more amino acid residues are substituted with conservative or nonconservative amino acids, (ii) variants in which one or more amino acids are added to or deleted from the polypeptide, (iii) variants in which one or more amino acids include a substituent group, and (iv) variants in which the described antibody or antigen-binding fragment is fused or conjugated with another peptide or polypeptide (e.g., a fusion partner, a protein tag) or other chemical moiety, that may confer useful properties to the antibody or antigen-binding fragment, such as, for example, an epitope for an antibody, a polyhistidine sequence, a biotin moiety and the like. Polypeptide compounds described herein may include variants in which amino acid residues from one species are substituted for the corresponding residue in another species, either at the conserved or nonconserved positions. In other embodiments, amino acid residues at nonconserved positions are substituted with conservative or nonconservative residues. Amino acid substitutions are generally based on the relative similarity of the amino acid side-chain substituents, for example, their hydrophobicity, hydrophilicity, charge, size, and the like.

Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group I (Ala, Ser, Thr, Pro, Gly); Group II (Asp, Asn, Glu, Gln); Group III (His, Arg, Lys); Group IV (Met, Leu, Ile, Val, Cys); Group V (Phe, Thy, Trp).

Accordingly, embodiments of the polypeptide compound can include variants having about 50%, 60%, 70%, 80%, 90%, 91%, 92%, 93%, 94%, 95%, 96%, 97%, 98%, 99%, or more sequence identity to the described polypeptide compound.

In some embodiments of methods provided herein, the methods further comprise administering to the subject one or more additional agents effective to treat the intended condition or disease. Such agents may comprise, without limitation, cholesterol-lowering drugs (e.g., statins, fibrates, inhibitors of proprotein convertase subtilisin/kexin type 9), blood pressure-lowering therapies (e.g., angiotensin-converting enzyme (ACE) inhibitors and angiotensin II receptor blockers (ARBs)), antiinflammatory agents, anti-thrombotic agents, anti-coagulant agents, inhibitors of the renin-angiotensin aldosterone system (RAAS inhibitors), beta-adrenergic blockers, calcium channel blockers, blood sugar reducing medications (e.g., metformin, insulin, glucose-dependent insulinotropic polypeptide (GIP), and glucagon-like peptide 1 (GLP-1), sodium/glucose co-transporter 2 (SGLT2) inhibitors), and/or other treatment modalities of a non-pharmacological nature. When combination therapy is used, the IL-6 inhibitor(s) and one additional agent(s) may be in the form of a single composition or multiple compositions, and the treatment modalities can be administered concurrently, sequentially, or through some other regimen. A combination therapy can have an additive or synergistic effect.

Other suitable agents that can be used in combination with the IL-6 inhibitors described herein include but are not limited to those Wnt inhibitors described in U.S. Pat. Nos. 11,369,609 and 9,045,416, which is hereby incorporated by reference in its entirety.

For example, a Wnt inhibitor to be used in combination with the the IL-6 inhibitors described herein may comprise a compound having the structure according to formula (I):

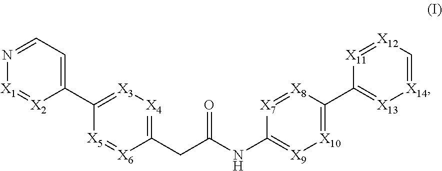

wherein X₁ and X₂ are selected from N and CR;
one of X₃, X₄, X₅ and X₆ is N and others are selected from N and CR;
one of X₇, X₈, X₉ and X₁₀ is N and others are selected from N and CR;
one of X₁₁, X₁₂, X₁₃ and X₁₄ is N and others are selected from N and CR, and
R is independently at each occurrence selected from hydrogen, halo, cyano, methyl, difluoromethyl, and trifluoromethyl,
   or a pharmaceutically acceptable salt thereof.

In some embodiments of the compound described above, one of X₃, X₄, X₅ and X₆ is N and the others are CR. In some embodiments, one of X₇, X₈, X₉ and X₁₀ is N and the others are CR. In some embodiments, two of X₁₁, X₁₂, X₁₃ and X₁₄ are N and the others are CR. In some embodiments, X₁ is CR and R is methyl, and/or wherein X₅ is CR and R is methyl. In some embodiments, one or more of X₂ is CH, X₄ is CH, X₆ is CH, X₈ is CH, X₉ is CH, X₁₀ is CH, X₁₂ is CH, and X₁₃ is CH.

In a specific embodiment, the compound is a compound having the structure according to formula (I), wherein
X₁ is CR, wherein R is methyl; X₂ is CH;
X₃ is N; X₅ is CR, wherein R is methyl; X₄ and X₆ are each CH;
X₇ is N; X₈, X₉ and X₁₀ are each CH;
X₁₁ and X₁₄ are each N; X₁₂ and X₁₃ are each CH.

In a specific embodiment, a Wnt inhibitor to be used in combination with the the IL-6 inhibitors described herein may comprise a compound LGK974 having the structure

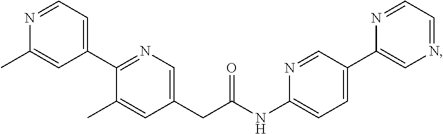 [SMILES: Cc1cc(-c2ncc(CC(=O)Nc3ccc(-c4cnccn4)cn3)cc2C)ccn1]

or a pharmaceutically acceptable salt thereof.

LGK974 can inhibit PORCN, which without wishing to be bound by theory is understood to be required for the palmitoylation of Wnt ligands. LGK974 may inhibit one or more of the following Wnt ligands: Wnt1, Wnt2, Wnt2B, Wnt3, Wnt3a, Wnt4, Wnt5a, Wnt5B, Wnt6, Wnt7a, Wnt7b, Wnt8a, Wnt8b, Wnt9a, Wnt9b, Wnt10a, Wnt10b, Wnt11, Wnt16. LGK974 may also inhibit phosphorylation of LRP5 and LPR6.

Chemical structures herein are drawn according to the conventional standards known in the art. Thus, where an atom, such as a carbon atom, as drawn appears to have an unsatisfied valency, then that valency is assumed to be satisfied by a hydrogen atom even though that a hydrogen atom is not necessarily explicitly drawn. Hydrogen atoms should be inferred to be part of the compound.

As would be understood by a person of ordinary skill in the art, the recitation of a compound is intended to include salts, solvates, oxides, and inclusion complexes of that compound as well as any stereoisomeric form, or a mixture of any such forms of that compound in any ratio. Compounds described herein include, but are not limited to, their optical isomers, racemates, and other mixtures thereof. In those situations, the single enantiomers or diastereomer, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

Thus, in accordance with some embodiments of the disclosure, a compound as described herein, including in the contexts of pharmaceutical compositions and methods of treatment is provided as the salt form. A “pharmaceutically acceptable salt” of a compound means a salt of a compound that is pharmaceutically acceptable. Desirable are salts of a compound that retain or improve the biological effectiveness and properties of the free acids and bases of the parent compound as defined herein or that take advantage of an intrinsically basic, acidic or charged functionality on the molecule and that are not biologically or otherwise undesirable. Examples of pharmaceutically acceptable salts are also described, for example, in Berge et al., “Pharmaceutical Salts”, J. Pharm. Sci. 66, 1-19 (1977). Non-limiting examples of such salts include: (1) acid addition salts, formed on a basic or positively charged functionality, by the addition of inorganic acids such as hydrochloric acid, hydrobromic acid, hydroiodic acid, sulfuric acid, sulfamic acid, nitric acid, phosphoric acid, carbonate forming agents, and the like; or formed with organic acids such as acetic acid, propionic acid, lactic acid, oxalic, glycolic acid, pivalic acid, t-butylacetic acid, β-hydroxybutyric acid, valeric acid, hexanoic acid, cyclopentanepropionic acid, pyruvic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, cyclohexylaminosulfonic acid, benzenesulfonic acid, sulfanilic acid, 4-chlorobenzenesulfonic acid, 2-napthalenesulfonic acid, 4-toluenesulfonic acid, camphorsulfonic acid, 3-phenyl propionic acid, lauryl sulphonic acid, lauryl sulfuric acid, oleic acid, palmitic acid, stearic acid, lauric acid, embonic (pamoic) acid, palmoic acid, pantothenic acid, lactobionic acid, alginic acid, galactaric acid, galacturonic acid, gluconic acid, glucoheptonic acid, glutamic acid, naphthoic acid, hydroxynapthoic acid, salicylic acid, ascorbic acid, stearic acid, muconic acid, and the like; (2) base addition salts, formed when an acidic proton present in the parent compound either is replaced by a metal ion, including, an alkali metal ion (e.g., lithium, sodium, potassium), an alkaline earth ion (e.g., magnesium, calcium, barium), or other metal ions such as aluminum, zinc, iron and the like; or coordinates with an organic base such as ammonia, ethylamine, diethylamine, ethylenediamine, N,N′-dibenzylethylenediamine, ethanolamine, diethanolamine, triethanolamine, tromethamine, N-methylglucamine, piperazine, chloroprocain, procain, choline, lysine and the like.

Pharmaceutically acceptable salts may be synthesized from a parent compound that contains a basic or acidic moiety, by conventional chemical methods. Generally, such salts are prepared by reacting the free acid or base forms of compounds with a stoichiometric amount of the appropriate base or acid in water or in an organic solvent, or in a mixture of the two. Salts may be prepared in situ, during the final isolation or purification of a compound or by separately reacting a compound in its free acid or base form with the desired corresponding base or acid, and isolating the salt thus formed. The term “pharmaceutically acceptable salts” also include zwitterionic compounds containing a cationic group covalently bonded to an anionic group, as they are “internal salts”. It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

The pharmaceutical compositions of the present disclosure may comprise the compounds described herein and a pharmaceutically acceptable carrier or excipient. Pharmaceutically acceptable carriers can include a physiologically acceptable compound that acts to, e.g., stabilize, or increase or decrease the absorption or clearance rate of a pharmaceutical composition. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of glycopeptides, or excipients or other stabilizers and/or buffers. Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. Detergents can also be used to stabilize or to increase or decrease the absorption of the pharmaceutical composition, including liposomal carriers. Pharmaceutically acceptable carriers and formulations are known to the skilled artisan and are described in detail in the scientific and patent literature, see e.g., the latest edition of Remington's Pharmaceutical Science, Mack Publishing Company, Easton, Pa. (“Remington's”). One skilled in the art would appreciate that the choice of a pharmaceutically acceptable carrier including a physiologically acceptable compound depends, for example, on the route of administration of the composition, and on its particular physio-chemical characteristics.

Compositions may be administered by any suitable means, for example, orally, such as in the form of pills, tablets, capsules, granules or powders; sublingually; buccally; parenterally, such as by subcutaneous, intravenous, intramuscular, intraperitoneal or intrasternal injection or using infusion techniques (e.g., as sterile injectable aqueous or non-aqueous solutions or suspensions); nasally, such as by inhalation spray, aerosol, mist, or nebulizer; topically, such as in the form of a cream, ointment, salve, powder, or gel; transdermally, such as in the form of a patch; transmucosally; or rectally, such as in the form of suppositories. The present compositions may also be administered in a form suitable for immediate release or extended release. Immediate release or extended release may be achieved by the use of suitable pharmaceutical compositions, or, particularly in the case of extended release, by the use of devices such as subcutaneous implants or osmotic pumps.

In various embodiments, the pharmaceutical composition is formulated for oral administration. Suitable forms for oral administration include, but are not limited to, tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically acceptable preparations. Tablets, capsules and the like generally contain the active ingredient in admixture with non-toxic pharmaceutically acceptable carriers or excipients which are suitable for the manufacture of tablets. These carriers or excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

Tablets, capsules and the like suitable for oral administration may be uncoated or coated using known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinyl acetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, using hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil. Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadeca ethyleneoxy cetanol), or condensation products of ethylene oxide with partial esters derived from fatty acids and a hexitol (e.g., polyoxy ethylene sorbitol rnonooleate), or condensation products of ethylene oxide with partial esters derived from fatty acids and hexitol anhydrides (e.g., polyethylene sorbitanmonooleate). The aqueous suspensions may also contain one or more preservatives.

Other suitable formulations for oral use include oily suspensions. Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known in the art.

Pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soybean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions of the disclosure can be produced in useful dosage units for administration by various routes including, among others, topical, oral, subcutaneous, intravenous, and intranasal administration.

The pharmaceutical compositions of the disclosure can also include other biologically active substances in combination with the compounds of the disclosure. Such additional biologically active substances can be also formulated as separate compositions and can be administered simultaneously or sequentially with the compounds of the disclosure. Non-limiting examples of useful biologically active substances include statins, niacin, bile-acid resins, fibric acid derivatives, cholesterol absorption inhibitors, and other lipid-lowering drugs.

The optimal therapeutically effective amount of a compound or composition of this disclosure may be determined experimentally, taking into consideration the exact mode of administration, the form in which the drug is administered, the indication toward which the administration is directed, the subject involved (e.g., body weight, health, age, sex, etc.), and the preference and experience of the physician or veterinarian in charge.

Following methodologies which are well-established in the art, effective doses and toxicity of the compounds and compositions of the present disclosure, which performed well in in vitro tests, can be determined in studies using small animal models (e.g., mice, rats) in which they have been found to be therapeutically effective and in which these drugs can be administered by the same route proposed for the human trials.

For any pharmaceutical composition used in the methods of the disclosure, dose-response curves derived from animal systems can be used to determine testing doses for administration to humans. In safety determinations for each composition, the dose and frequency of administration should meet or exceed those anticipated for use in any clinical trial.

As disclosed herein, the dose of the compounds or compositions of the present disclosure is determined to ensure that the dose administered continuously or intermittently will not exceed an amount determined after consideration of the results in test animals and the individual conditions of a patient. A specific dose naturally varies (and is ultimately decided according to the judgment of the practitioner and each patient's circumstances) depending on the dosage procedure, the conditions of a patient or a subject animal such as age, body weight, sex, sensitivity, feed, dosage period, drugs used in combination, seriousness of the disease, etc.

Toxicity and therapeutic efficacy of the compositions of the disclosure can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀.

The compounds the disclosure can be formulated for parenteral, oral, topical, transdermal, transmucosal, intranasal, buccal administration, or by any other standard route of administration. Parenteral administration includes, among others, intravenous (i.v.), subcutaneous (s.c.), intraperitoneal (i.p.), intramuscular (i.m.), subdermal (s.d.), intradermal (i.d.), intra-articular, intra-synovial, intra-arteriole, intraventricular, intrathecal, intrasternal, intrahepatic, intralesional, or intracranial administration, by direct injection, via, for example, bolus injection, continuous infusion, or gene gun. A preferred route of administration according to the present disclosure will depend primarily on the indication being treated and includes, among others, topical, oral, subcutaneous, intravenous, and intranasal administration.

Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use. Suitable formulations for parenteral administration may contain substances which increase viscosity, for example, sodium carboxymethyl cellulose, sorbitol, and/or dextran. Optionally, the formulation may also contain stabilizers. Additionally, the compounds of the present disclosure may also be administered encapsulated in liposomes. The compounds, depending upon their solubilities, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

In specific embodiments, the compounds and/or compositions of the present disclosure are formulated for oral administration. For oral administration, the formulations of the disclosure can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. The compositions of the disclosure can be also introduced in microspheres or microcapsules, e.g., fabricated from poly glycolic acid/lactic acid (PGLA) (see, U.S. Pat. Nos. 5,814,344; 5,100,669 and 4,849,222; PCT Publication Nos. WO 95/11010 and WO 93/07861). Liquid preparations for oral administration can take the form of, for example, solutions, syrups, emulsions or suspensions, or they can be presented as a dry product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For administration by inhalation, the therapeutics according to the present disclosure can be conveniently delivered in the form of an aerosol spray presentation from pressurized packs or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In addition to the formulations described previously, the compositions can also be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

### EXAMPLES

The following examples are provided to further describe some of the embodiments disclosed herein. The examples are intended to illustrate, not to limit, the disclosed embodiments.

### Example 1. Endothelial GR Deficiency Results in a Fibrogenic Phenotype in the Kidneys of Diabetic Mice

The streptozotocin (STZ)-induced diabetic CD-1 mouse is the established mouse model to study diabetic kidney disease²⁹⁻³¹, as the kidney fibrosis phenotype is dependent upon mouse strain specificity³⁰. Though STZ-induced diabetic CD-1 mice and diabetic C57BL/6 mice demonstrate similar blood glucose levels, the kidneys of diabetic CD-1 mice have been shown to have higher rates of EndMT and more severe fibrosis when compared to the kidneys of diabetic C57BL/6 mice^{29,32}. Therefore, diabetic CD-1 mice are considered a pro-fibrotic strain while diabetic C57B/L6 mice are considered to be a less-fibrotic strain^{32,33}.

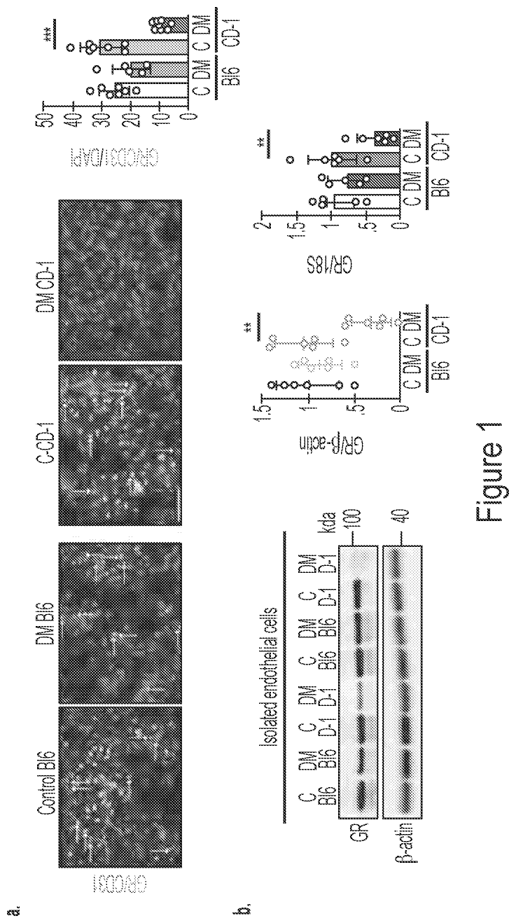

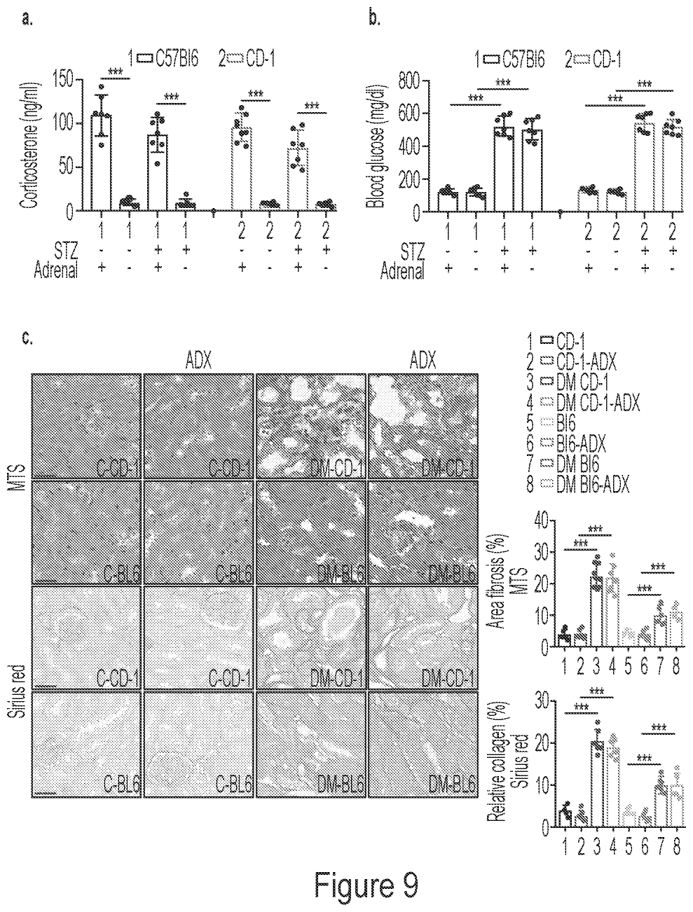

Diabetic CD-1 mouse kidneys displayed significant suppression of GR compared to those from diabetic C57BL/6 mice as assessed by immunofluorescent staining (FIG. 1A). Moreover, ECs isolated from the kidneys of diabetic CD-1 mice showed dramatic suppression in both the GR protein and mRNA levels when compared to the diabetic C57BL/6 mice and the non-diabetic controls of both genotypes (FIG. 1B). The effect of corticosterone deficiency on the progression of kidney fibrosis was evaluated by pursuing bilateral adrenalectomy in both strains of mice. The efficacy of the surgical procedure was verified by substantially suppressed corticosterone levels in all mice studied (FIG. 9A); adrenalectomy did not affect glycemia (FIG. 9B). Adrenalectomy did not cause any significant difference in either the extent of fibrosis or the collagen expression level (FIG. 9C), suggesting that the global suppression of corticosterone did not influence the observed fibrosis phenotype.

### Example 2. Loss of EC GR Worsens Kidney Fibrosis

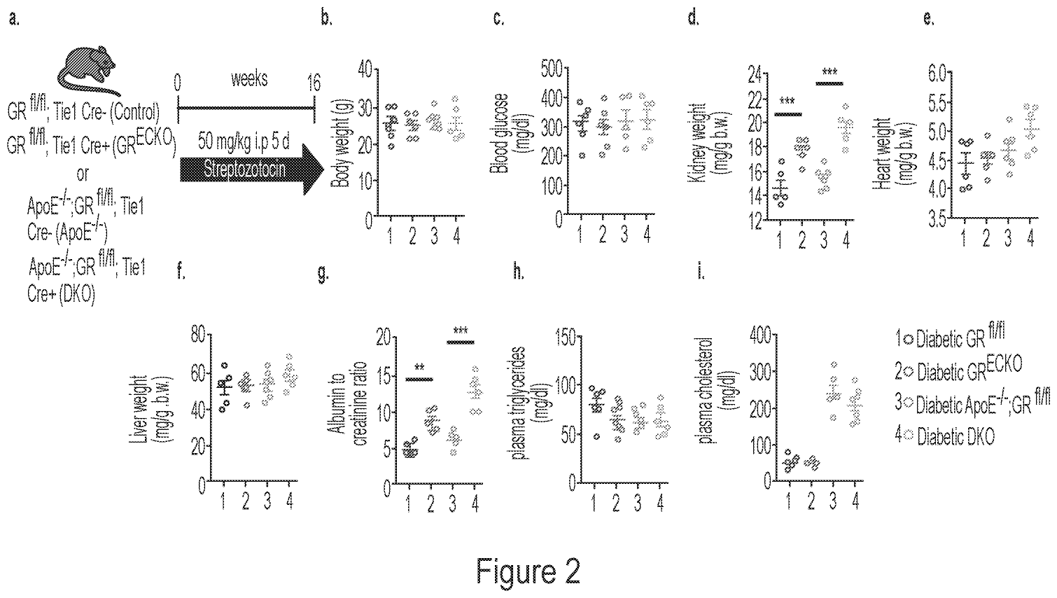

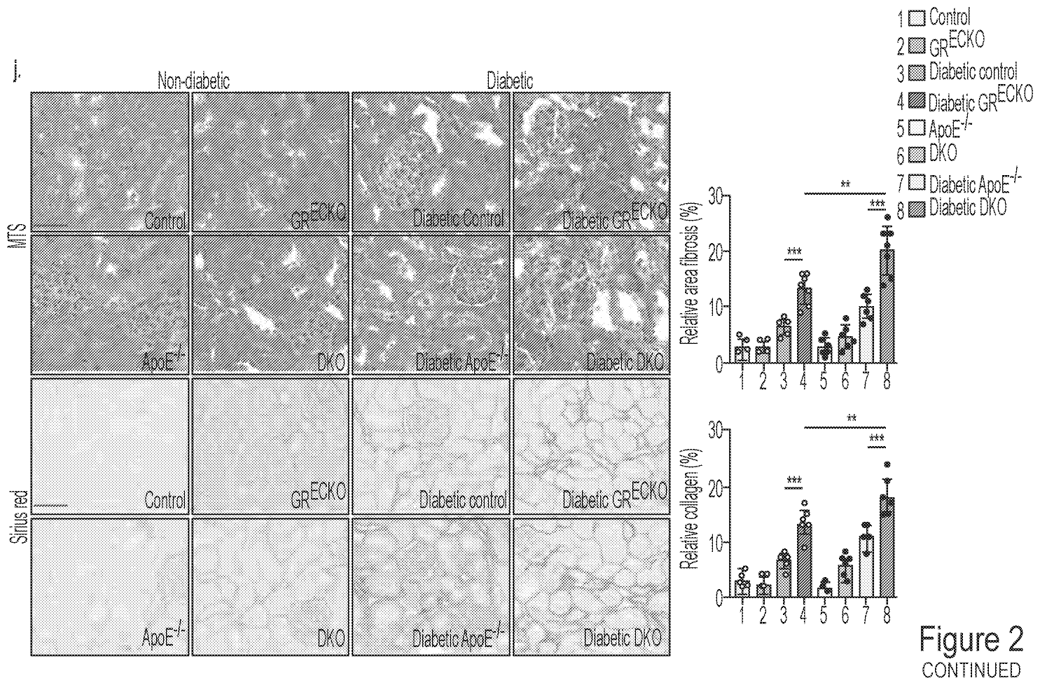

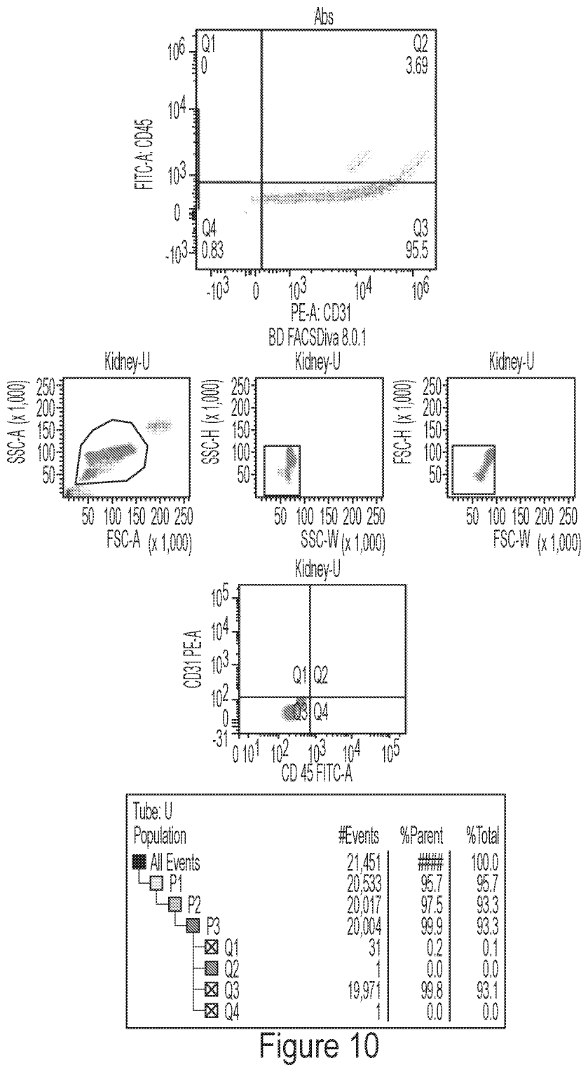

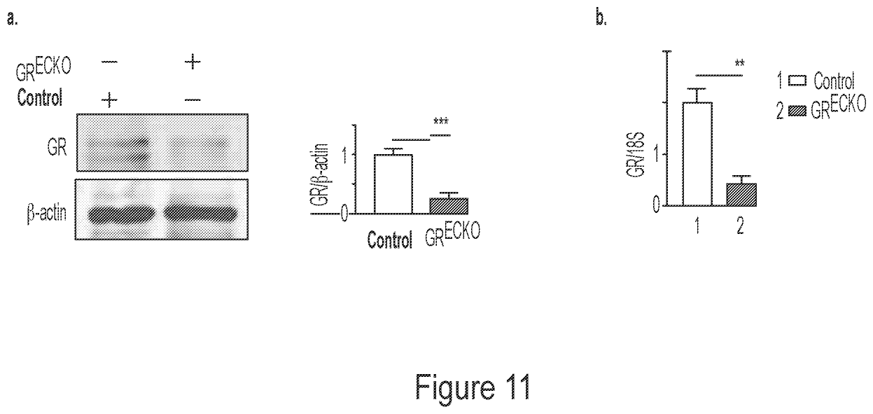

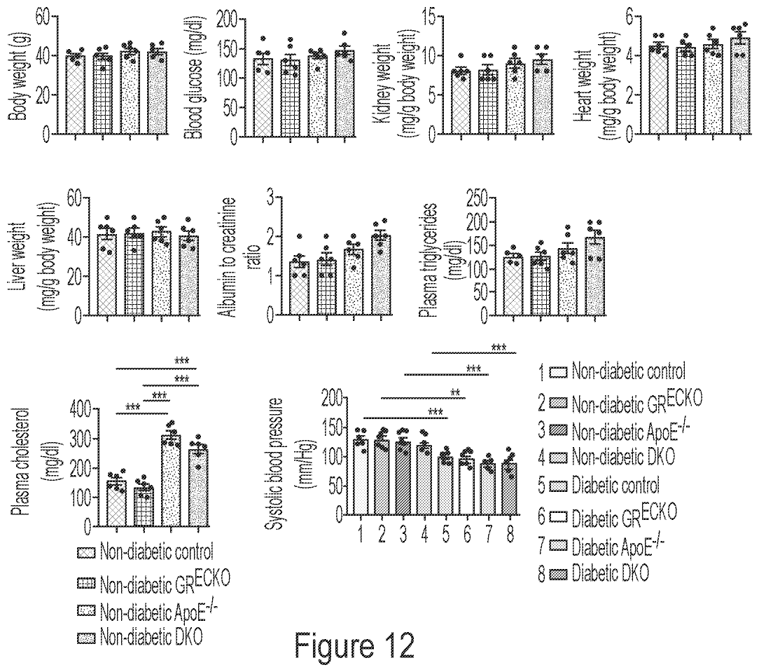

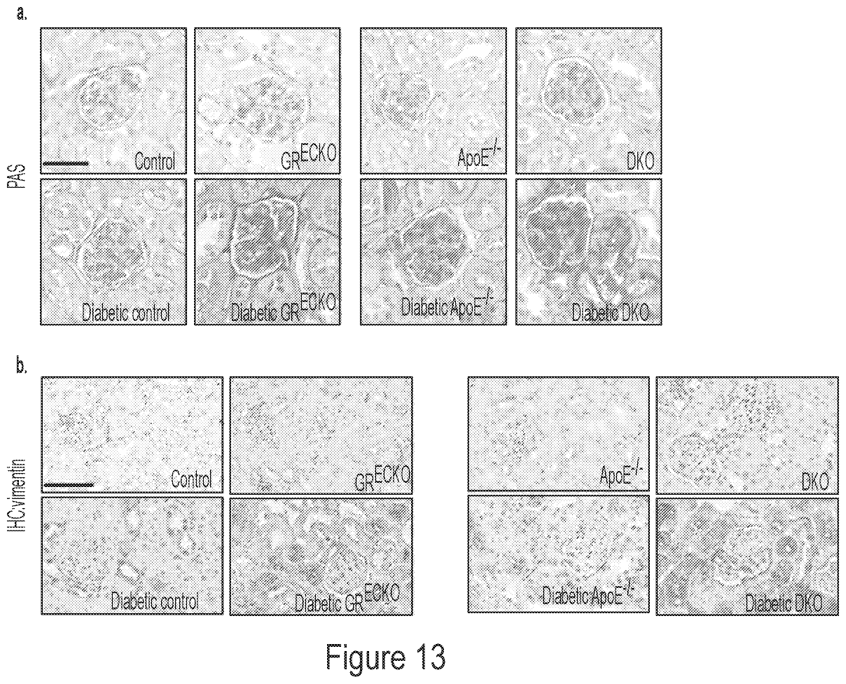

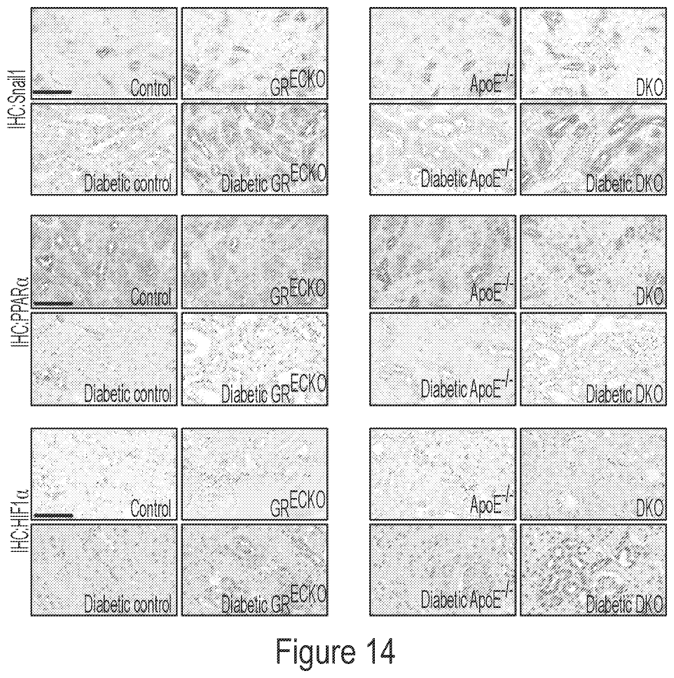

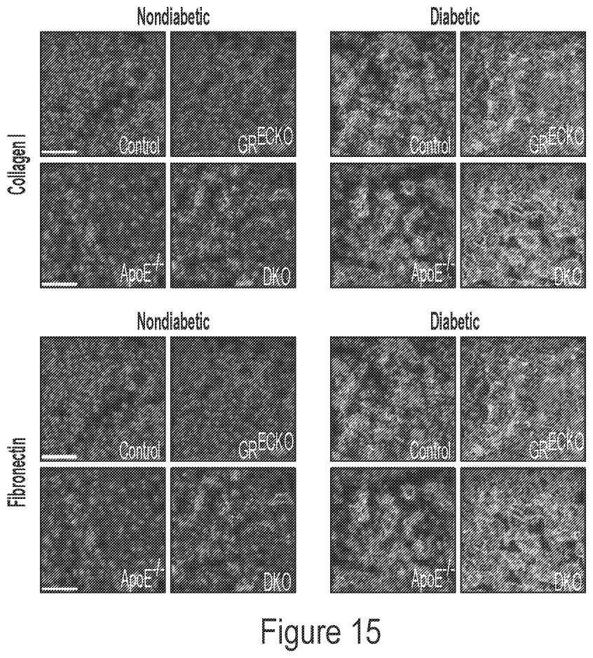

To verify efficient GR excision from ECs in the kidneys of endothelial GR knockout (GR^{ECKO}) mice, the inventors performed Western blot and qPCR for GR in isolated kidney endothelial cells. Flow cytometry analysis demonstrated that kidney EC isolation was accomplished with ˜95% purity (FIG. 10). As shown in FIGS. 11A-11B, mRNA and protein levels were significantly diminished in ECs from GR^{ECKO} mice, as expected. Diabetes was produced by injecting 5 consecutive low doses of STZ (50 mg/kg/day IP) in 8-week-old GR^{ECKO} (GR^{fl/fl}; Tie1 Cre+) and Cre− littermate controls (GR^{fl/fl}) and GR^{fl/fl}; Tie1 Cre+/Apoe^{-/-} (DKO) mice and Cre− littermates (GR^{fl/fl}; Apoe^{-/-}) (FIG. 2A). Animals were monitored for 4 months post-STZ treatment before sacrifice. At the time of sacrifice, non-diabetic and diabetic GR^{ECKO} and DKO mice and their littermate controls had no significant change in body weight, blood glucose, heart weight, liver weight, triglycerides or cholesterol; however, diabetic GR^{ECKO} and diabetic DKO had relatively higher kidney weight and albumin-to-creatinine ratios when compared to their respective diabetic controls. (FIGS. 2B-2I, FIG. 12). The systolic blood pressure of diabetic mice of all genotypes was ˜20 mm Hg lower than the corresponding non-diabetic controls, presumably due to the massive polyuria induced by diabetes, but not significantly different otherwise (FIG. 12). Diabetic DKO had significantly higher kidney weight and albumin-to-creatinine ratios when compared to diabetic GR^{ECKO} Renal fibrosis was assessed by histologic analysis of kidney sections from all genotypes. Diabetic GR^{ECKO} mice exhibited a higher relative area of fibrosis, higher relative collagen deposition and more severe glomerulosclerosis at the 4-month timepoint when compared to diabetic littermate controls (FIG. 2J, FIG. 13A). Diabetic DKO exhibited greatly increased relative area of fibrosis and relative collagen deposition when compared to diabetic Apoe^{-/-} controls and diabetic GR^{ECKO} (FIG. 2J). Diabetic GR^{ECKO} mice exhibited higher expression of vimentin, snail1, and HIF1α and lower expression of PPARα at the 4-month timepoint when compared to diabetic littermate controls (FIG. 13B; FIG. 14). A similar pattern was observed in diabetic DKO when compared to diabetic Apoe^{-/-} controls (FIG. 13B; FIG. 14). Immunofluorescence data showed higher collagen I and fibronectin deposition in the kidneys of diabetic animals with GR^{ECKO}, with the highest deposition observed in DKO mice (FIG. 15).

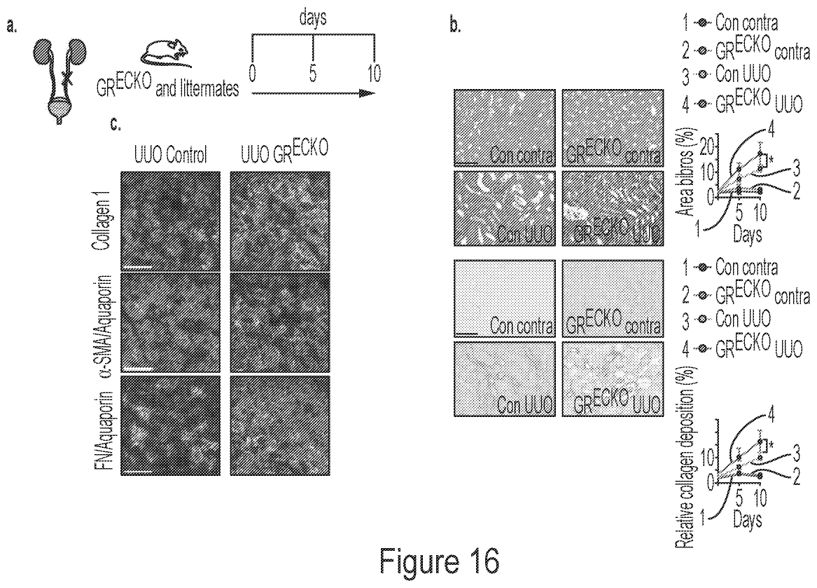

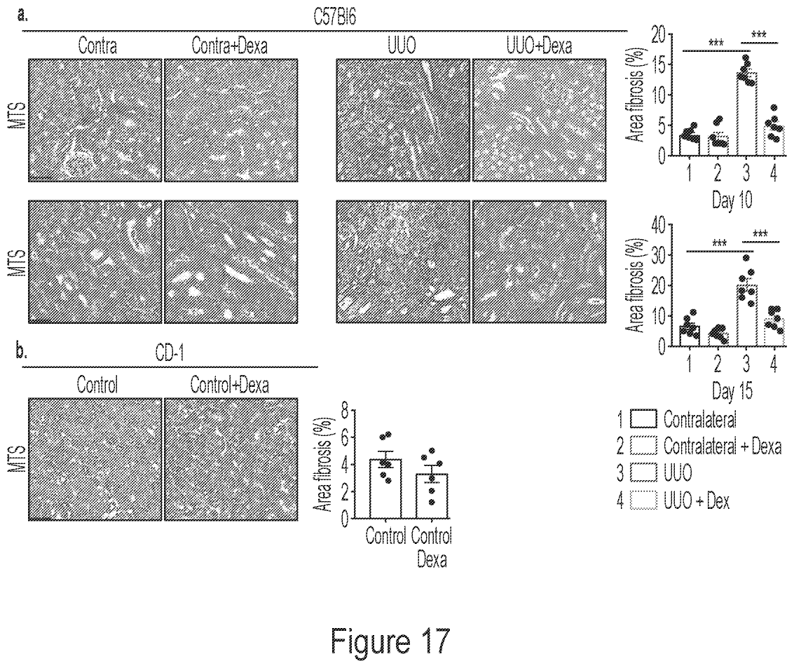

In order to test the role of endothelial GR in non-diabetic fibrosis, unilateral ureteral obstruction (UUO) in 8-week-old GR^{ECKO} and control littermates was performed (FIG. 16A). There was no significant difference in renal fibrosis between contralateral kidneys of controls and GR^{ECKO} mice. However, UUO kidneys from GR^{ECKO} mice showed a greater relative area of fibrosis and greater collagen deposition when compared to UUO kidneys of littermate controls FIG. 16B). Immunofluorescence staining revealed higher collagen I, αSMA, and fibronectin deposition in the UUO kidneys of GR^{ECKO} when compared to UUO kidneys of control littermates (FIG. 16C). Administration of the GR agonist dexamethasone abolished the renal fibrogenic phenotype in C57BL/6 mice subjected to UUO by day 10 (FIG. 17A). Dexamethasone treatment slightly suppressed the renal fibrosis in contralateral kidneys of C57BL/6 mice at day 15 but did not cause any remarkable alteration in nondiabetic control CD-1 mice (FIGS. 17A-17B).

### Example 3. Endothelial GR Loss Reprograms Cytokine and Chemokine Homeostasis

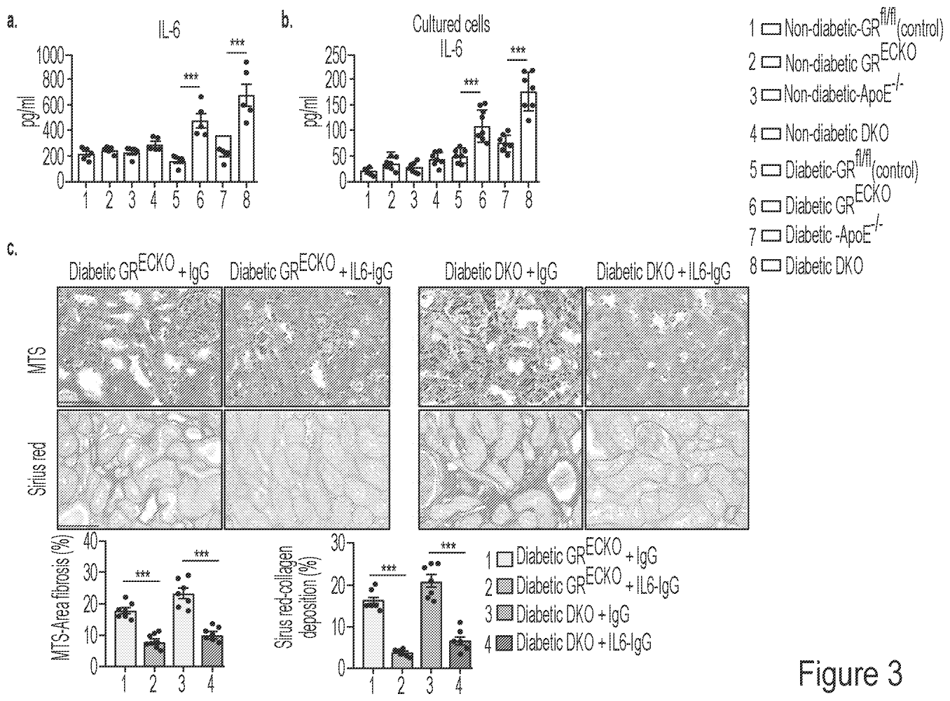

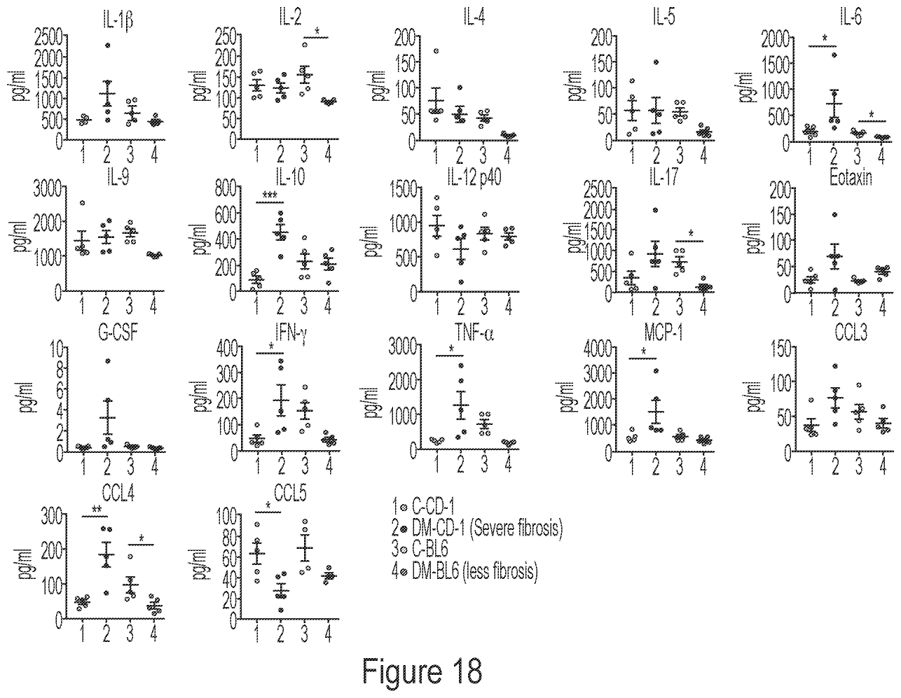

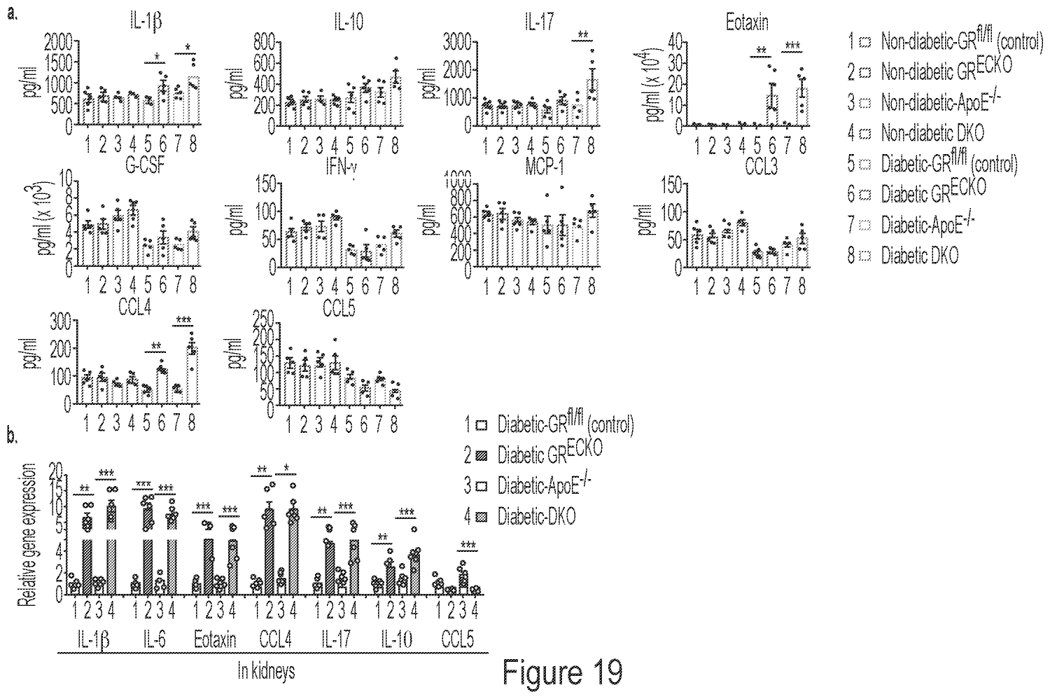

Inflammation is a key factor during the fibroblast activation process in the kidneys of diabetic mice^{34,35} and disruption of cytokine and chemokine homeostasis contributes to the development of diabetic kidney disease³⁶⁻³⁸. To investigate whether there were derangements in homeostasis in the model, cytokine analysis in the plasma of diabetic mice with severe fibrosis (diabetic CD-1) and the plasma of diabetic mice with less severe fibrosis (diabetic C57BL/6) was performed. Diabetic CD-1 mice demonstrated higher levels of plasma IL-1β, IL-6, IL-10, IL-17, G-CSF, IFN-γ, TNF-α, MCP-1, CCL3 and CCL4 levels, however the CCL5 level was suppressed when compared to that of diabetic C57BL/6 mice (FIG. 18). The same cytokines were also analyzed in the plasma from diabetic and non-diabetic GR^{ECKO} mice and littermate controls and diabetic and non-diabetic DKO and Apoe^{-/-} controls. A similar pattern was observed in both genotypes in that IL-1β, IL-6, IL-10, eotaxin, G-CSF and CCL4 were significantly higher, while CCL5 was significantly lower, in the plasma of diabetic GR^{ECKO} and diabetic DKO mice when compared to the plasma of their respective diabetic control littermates (FIG. 3A; FIG. 19A). Similarly, mRNA gene expression analysis demonstrated that the level of IL-1β, IL-6, IL-10, IL-17, eotaxin, and CCL4 were significantly upregulated, while CCL5 was significantly downregulated, in the kidneys of diabetic GR^{ECKO} and diabetic DKO mice when compared to the diabetic kidneys of their respective control littermates (FIG. 19B), indicating more EC inflammation in mice lacking endothelial GR.

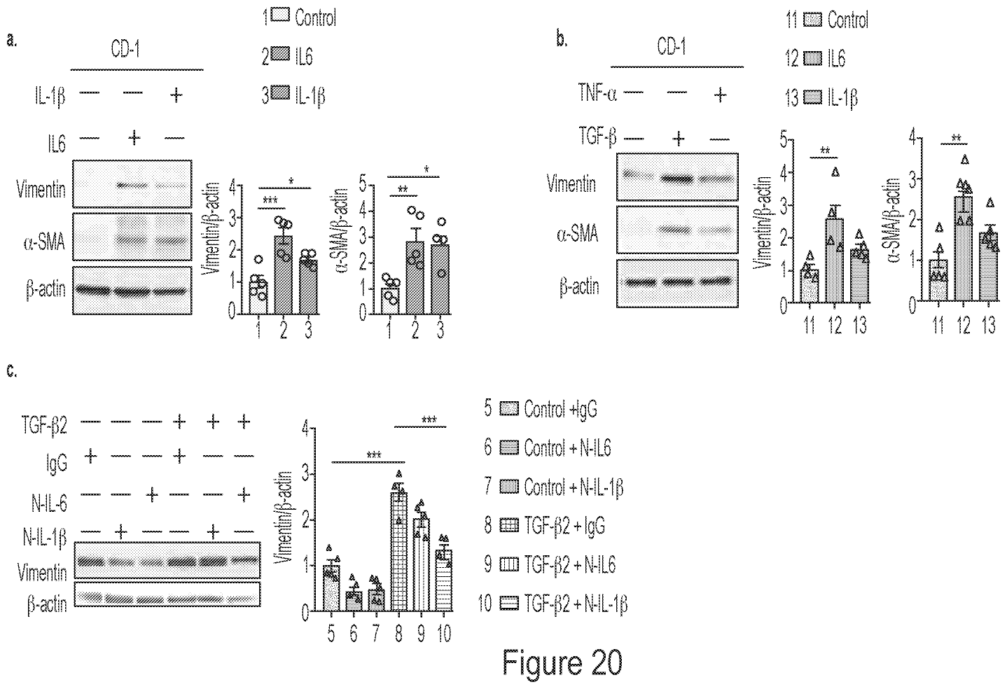

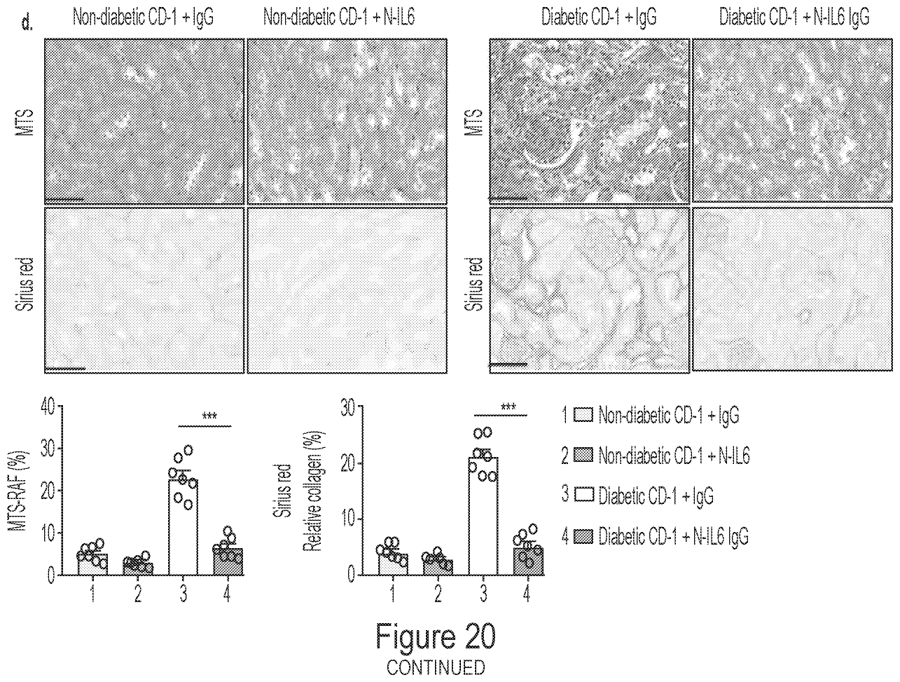

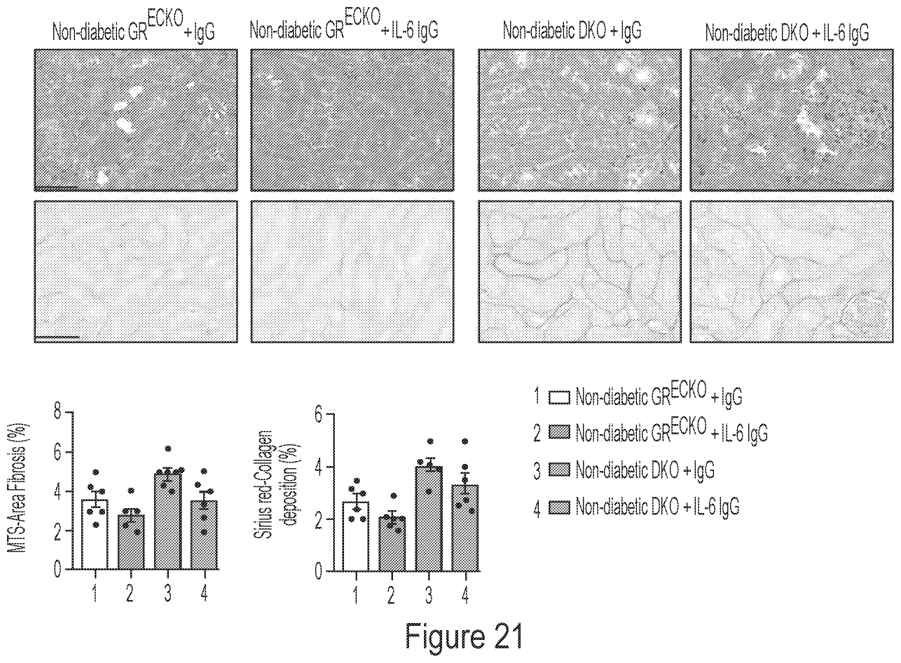

Stimulation of isolated kidney endothelial cells from CD-1 mice with recombinant IL-1β, IL-6, TNFα and TGFβ caused a significant increase in expression of the mesenchymal markers vimentin and αSMA (FIGS. 20A-20B); neutralization of IL-6 using IL-6 neutralizing antibody could suppress vimentin expression in TGFβ2-treated and untreated cells (FIG. 20C). To further investigate the role of IL-6 in diabetes-associated renal fibrosis, the inventors injected (IP) IL-6 neutralizing antibody in nondiabetic and diabetic CD-1 mice. In non-diabetic mice, IL-6 neutralization did not cause any significant difference; however, in diabetic mice, IL-6 neutralization resulted in a significant reduction of fibrosis and collagen deposition (FIG. 20D). In addition, isolated cells from diabetic GR^{ECKO} and DKO mice had higher IL-6 levels in culture media when compared to isolated cells from their respective diabetic control littermates (FIG. 3B). When IL-6 neutralization was performed in nondiabetic and diabetic GR^{ECKO} and DKO mice, it completely reversed the fibrogenic phenotype in diabetic animals when compared to respective IgG control-injected mice (FIG. 3C). There was no significant effect observed in non-diabetic mice (FIG. 21).

### Example 4. Canonical Wnt Signaling is a New Drug Target for the Action of Endothelial GR

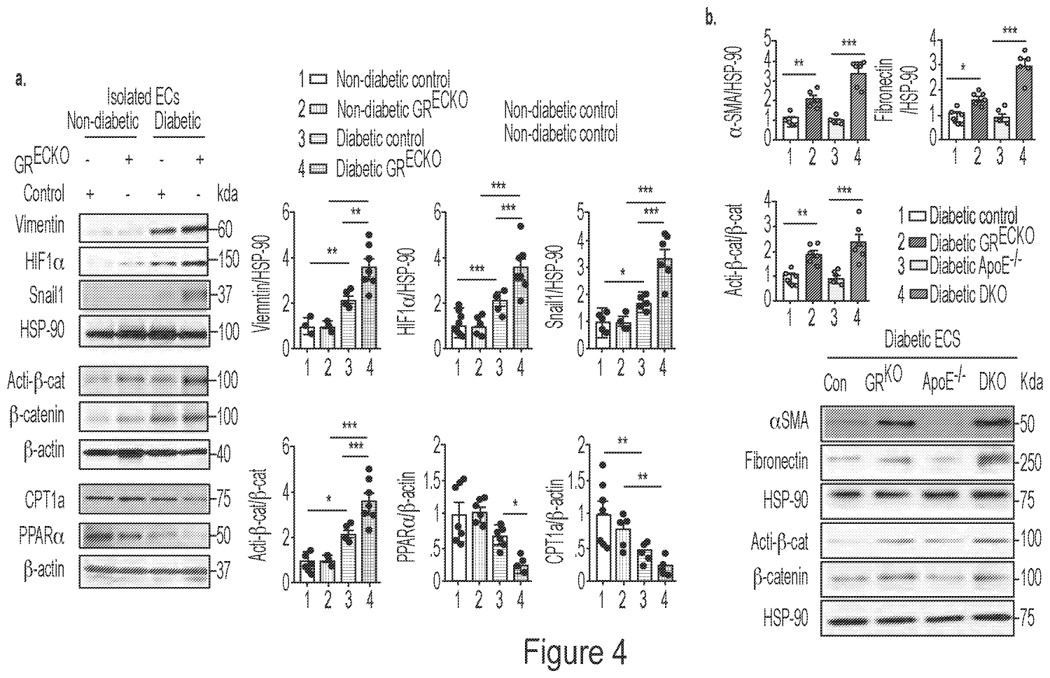

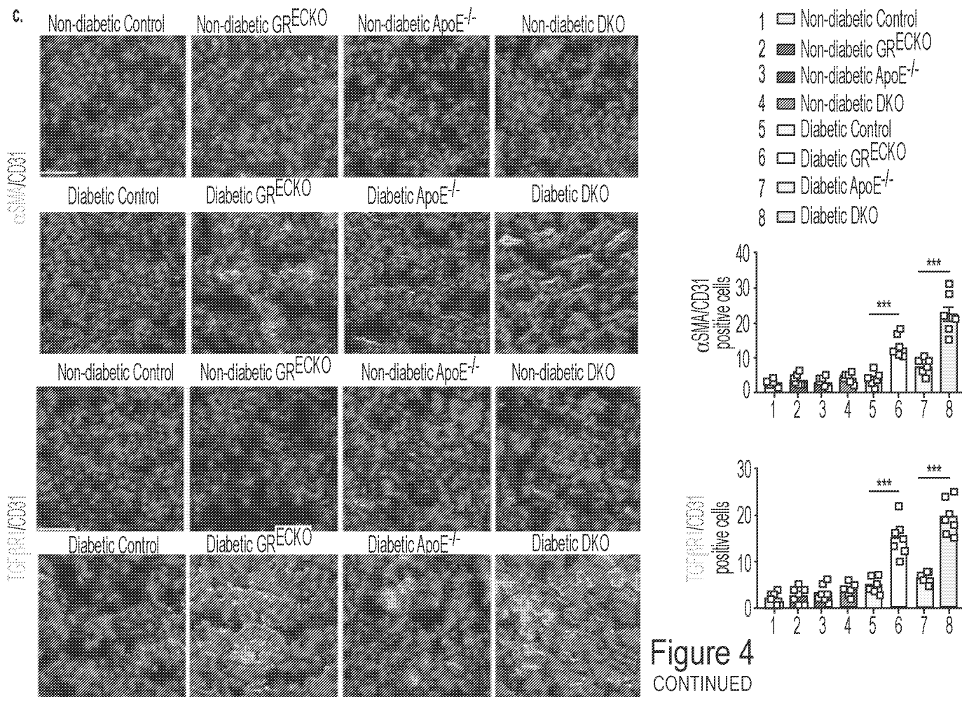

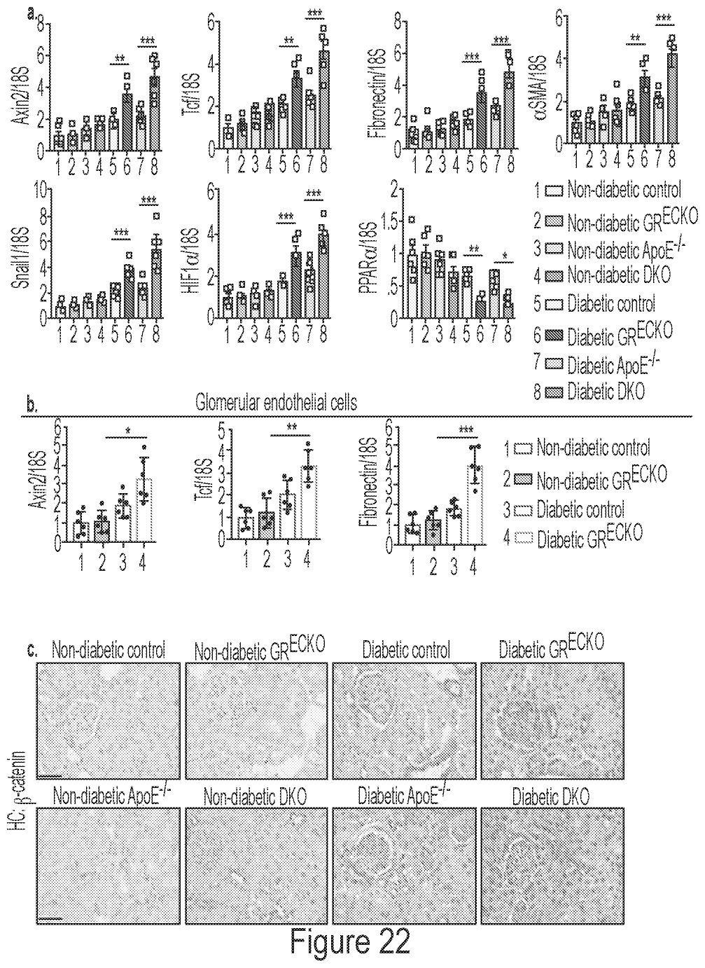

Given the recently described regulation of Wnt signaling by endothelial GR²⁷ as well as the recognized role of Wnt signaling in renal fibrosis³⁹, the inventors assessed the mRNA expression of Wnt-dependent genes and fibrogenic markers in ECs isolated from the kidneys of diabetic GR^{ECKO} and diabetic DKO mice and their diabetic littermate controls. The expression of Wnt-dependent genes and fibrogenic markers was upregulated in kidneys of diabetic GR^{ECKO} and diabetic DKO when compared to their respective controls. However, the kidneys of diabetic DKO mice showed the highest expression of both Wnt-dependent genes, such as axin2 and tcf and fibrogenic markers, such as αSMA andfibronectin (FIG. 14A). The expression of HIF1α and Snail1 was upregulated while PPARα expression was downregulated in ECs isolated from the kidneys of diabetic GR^{ECKO} and diabetic DKO mice when compared to diabetic controls (FIG. 22A). In ECs isolated from cultured glomeruli, the expression of Wnt-dependent genes axin2 and tcf and fibronectin was upregulated in those from the kidneys of diabetic GR^{ECKO} mice compared to littermate control mice and non-diabetic mice (FIG. 22B). Protein expression of β-catenin was also higher in the interstitial and glomerular compartments of diabetic GR^{ECKO} and diabetic DKO compared to their respective controls (FIG. 22C). Western blotting demonstrated higher protein expression of vimentin, HIF1α, snail1, and active β-catenin and decreased levels of PPARα and CPT1a in isolated ECs from diabetic GR^{ECKO} when compared to diabetic control littermates (FIG. 4A). Isolated ECs from diabetic DKO mice exhibited higher fibronectin, αSMA and active β-catenin levels when compared to littermate Apoe−/− and diabetic GR^{ECKO} mice (FIG. 4B).

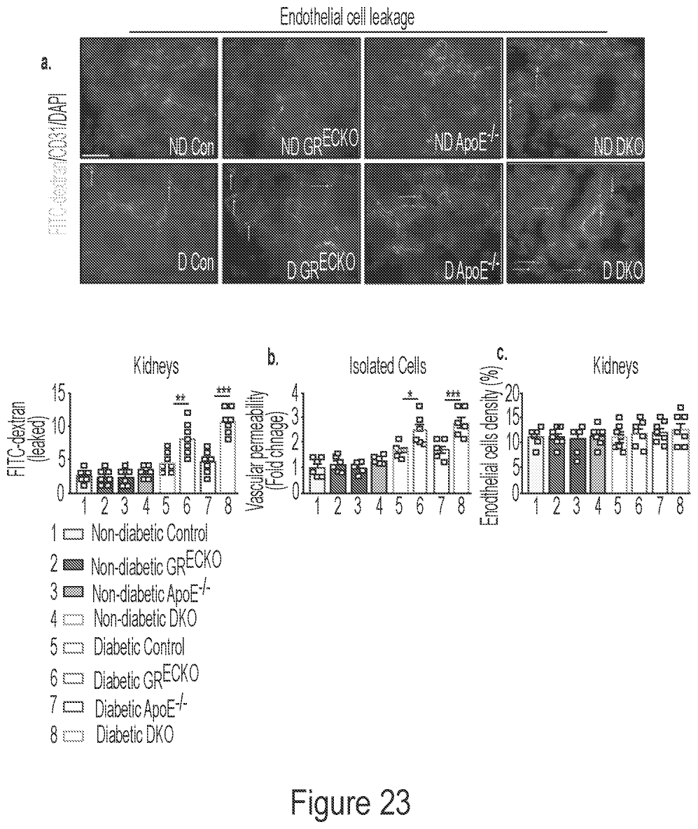

Using immunofluorescent co-staining, the same pattern was also observed, with diabetic GR^{ECKO} and diabetic DKO mice demonstrating higher levels of αSMA/CD31 and TGFβR1/CD31 co-staining in the kidneys when compared to their respective controls, suggestive of EndMT (FIG. 4C). There was no difference in the endothelial cell density of ECs from the kidneys of diabetic GR^{ECKO} and diabetic DKO when compared to control ECs; however, endothelial cell permeability, as demonstrated by FITC-dextran staining, was higher in kidneys and isolated cultured ECs from diabetic GR^{ECKO} and diabetic DKO when compared to their respective control littermates (FIGS. 23A-23C).

### Example 5. Inhibition of Canonical Wnt Signaling Improves Renal Fibrosis

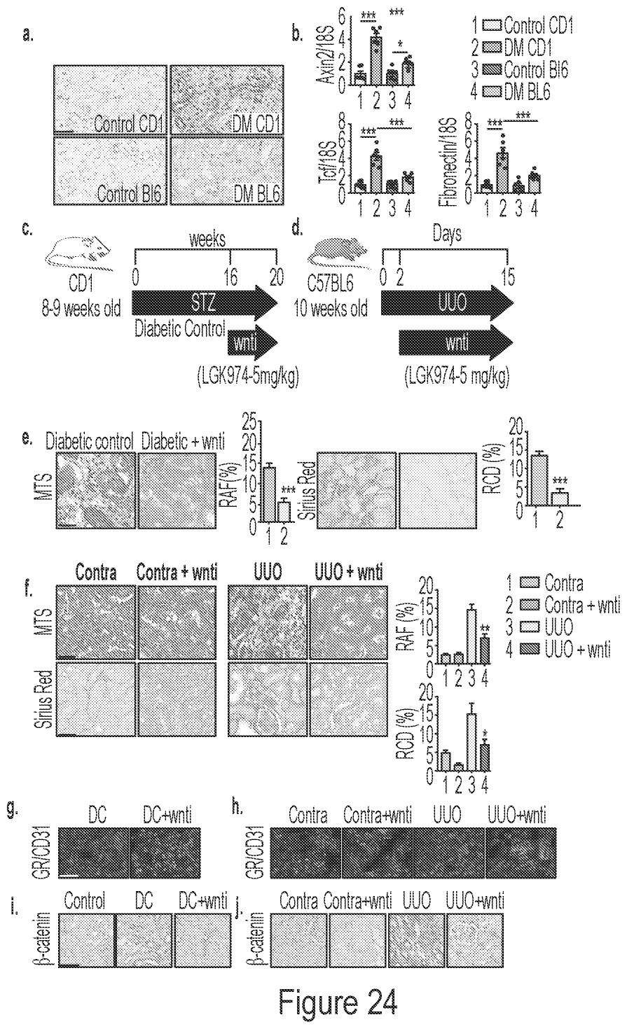

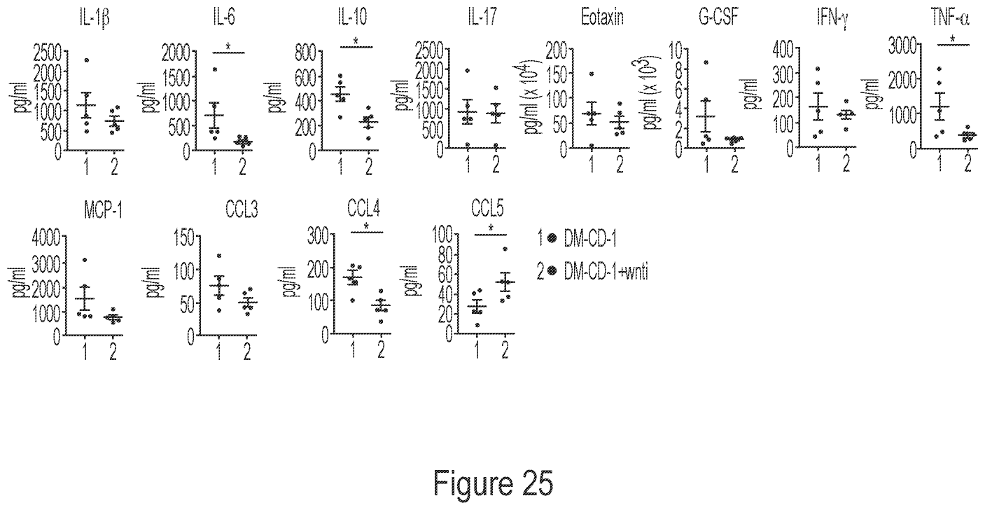

Diabetic CD-1 mouse kidneys displayed significantly higher expression of β-catenin, a marker of canonical Wnt signaling, compared to those from diabetic C57BL/6 mice as assessed by immunohistochemical staining (FIG. 24A). The kidneys of diabetic CD-1 mice also showed dramatically higher mRNA expression of Wnt-dependent genes and fibronectin when compared to those of the diabetic C57BL/6 mice and the non-diabetic controls of both genotypes (FIG. 24B). To determine whether inhibition of the Wnt signaling pathway could ameliorate the observed fibrosis, the inventors utilized LGK974, a small molecule inhibitor of all secreted Wnts⁴⁰. FIG. 24C-D depicts the schematic diagram showing the experimental protocol for LGK974 treatment in diabetic CD-1 and UUO mice. LGK974 greatly diminished the ECM deposition, relative area of fibrosis, collagen accumulation and glomerulosclerosis in both models used (FIGS. 24E-24F). Wnt inhibition also substantially restored endothelial GR and suppressed the level of β-catenin in diabetic and UUO mice (FIGS. 24G-24J). In diabetic CD-1 mice, LGK974 also significantly suppressed elevated levels of IL-1β, IL-6, IL-10, G-CSF, TNFα, MCP-1, and CCL4 while increasing the level of CCL5 (FIG. 25).

### Example 6. Wnt Inhibitor Partially Suppresses the Fibrogenic Phenotype in the Kidneys of Diabetic GR^{ECKO}

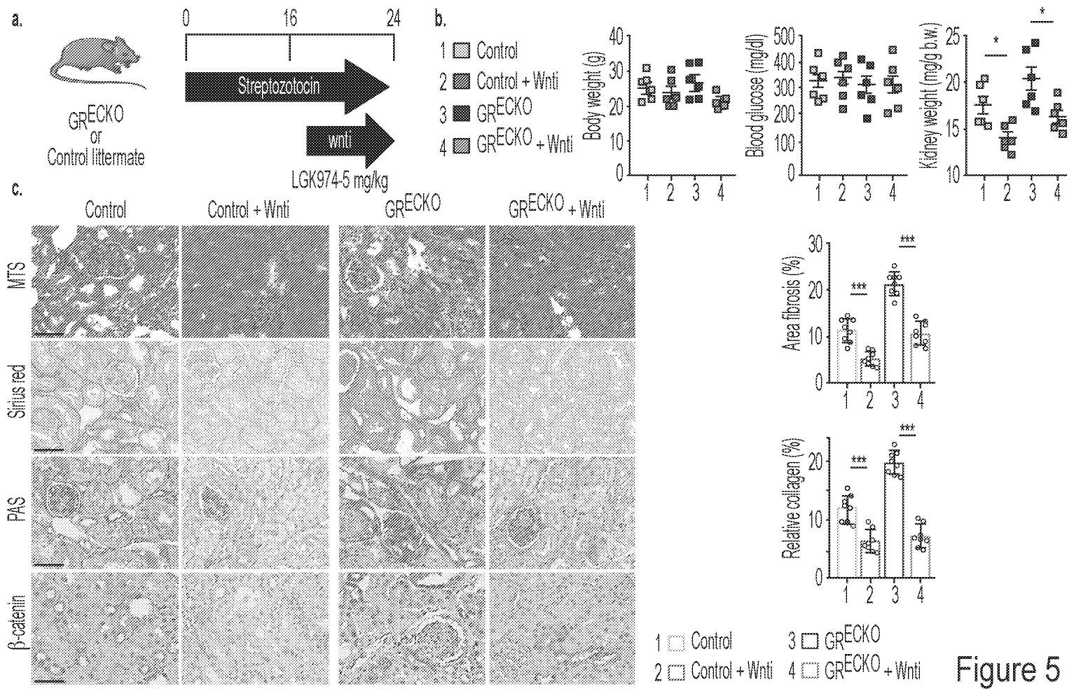

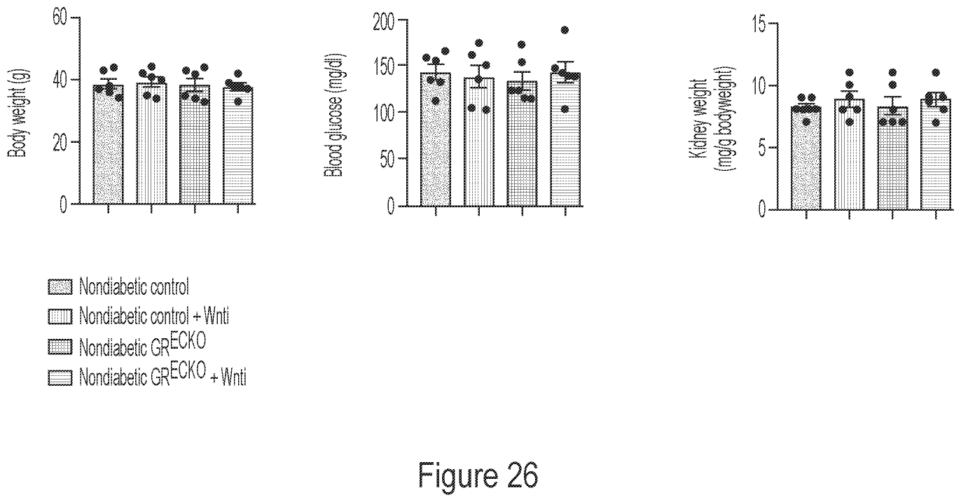

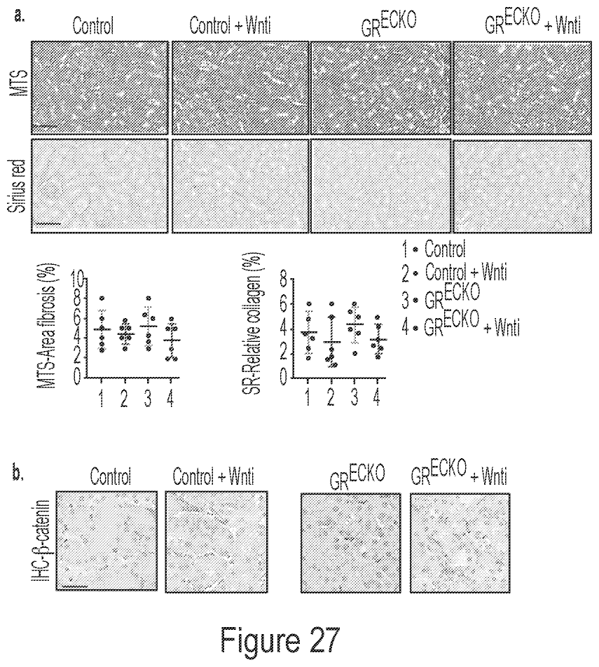

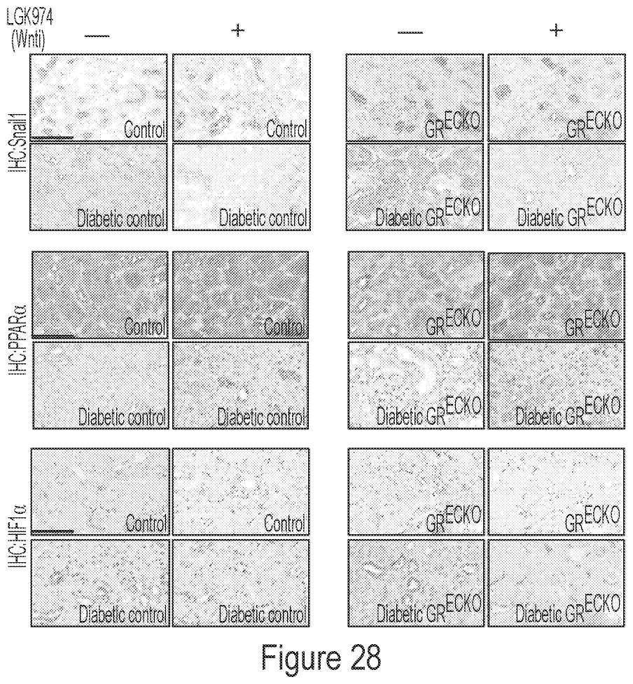

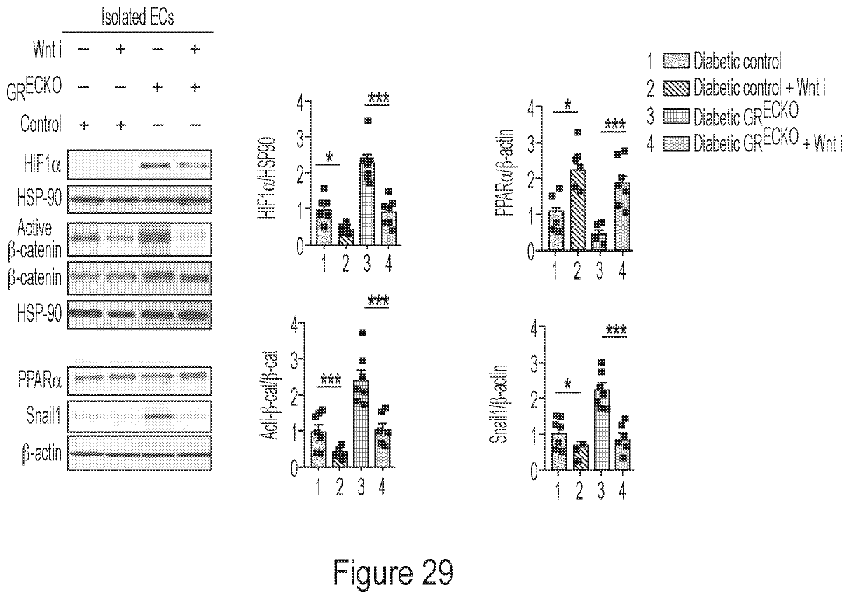

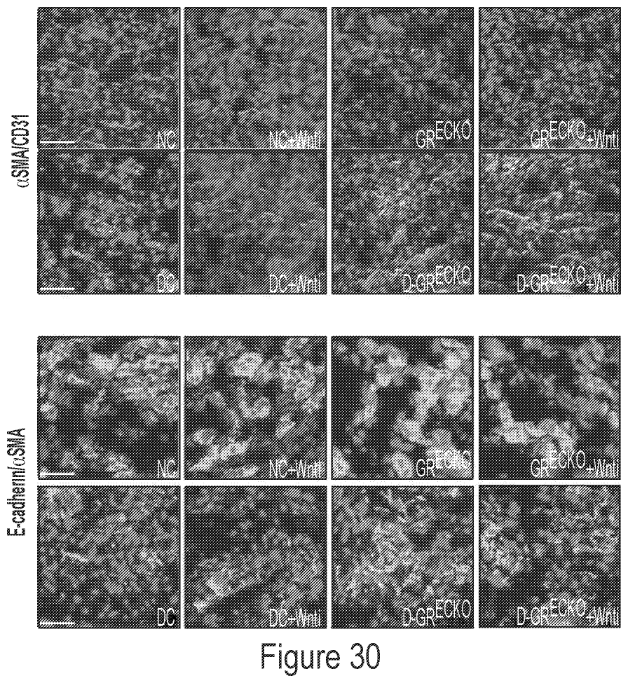

To determine whether Wnt inhibition could mitigate the renal fibrosis observed in diabetic GR^{ECKO} mice, a cohort of animals was treated with LGK974. At the age of 8 weeks, control and GR^{ECKO} mice were injected with STZ 50 mg/kg for five consecutive days. Sixteen weeks after injection, LGK974 was administered by oral gavage for eight additional weeks (FIG. 5A). At the time of sacrifice, there were no significant differences in body weight or blood glucose among the groups (FIG. 5B; FIG. 26). However, a significant reduction in kidney weight was observed in the Wnt-inhibitor treated diabetic GR^{ECKO} and diabetic control mice (FIG. 5B). Wnt inhibition clearly improved the relative area of fibrosis, relative collagen deposition and tubular damage in diabetic control mice; this effect was less pronounced, though still significant in diabetic GR^{ECKO} mice (FIG. 5C). Wnt inhibition did not result in any remarkable change in the renal fibrogenic phenotype or β-catenin level in nondiabetic control mice or nondiabetic GR^{ECKO} when compared to their untreated nondiabetic controls (FIG. 27). Wnt inhibition suppressed the expression of Snail1 and HIF1α and significantly increased the level of PPARα in the kidneys of diabetic GR^{ECKO} (FIG. 28). A similar effect was observed in isolated ECs (FIG. 29). Wnt inhibition also significantly suppressed EndMT (CD31/αSMA double positive cells) in diabetic control mice; this effect was less pronounced in diabetic GR^{ECKO} mice (FIG. 30). However, Wnt inhibition significantly reduced the level of EMT (E-cadherin/αSMA double positive cells) in control and diabetic GR^{ECKO} mice (FIG. 30).

### Example 7. Metabolic Reprogramming by Loss of Endothelial GR Accelerates Renal Fibrosis

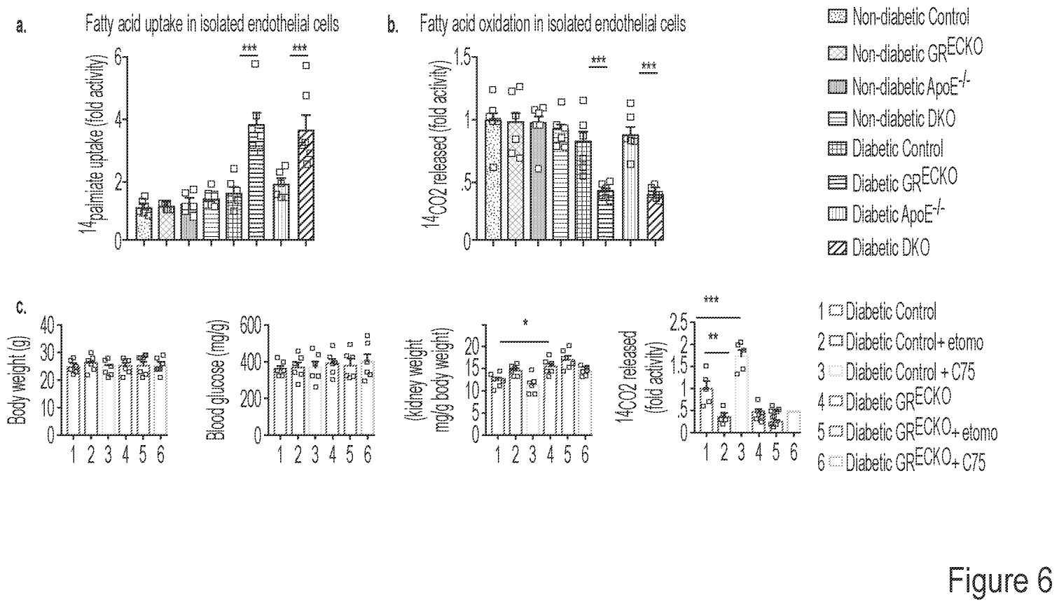

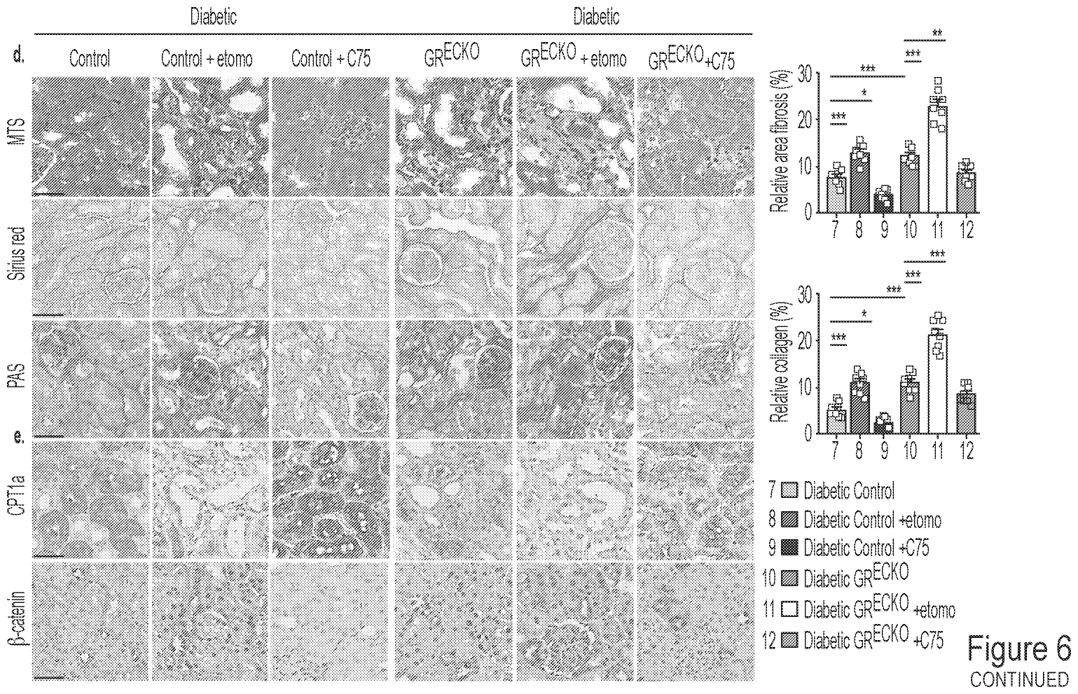

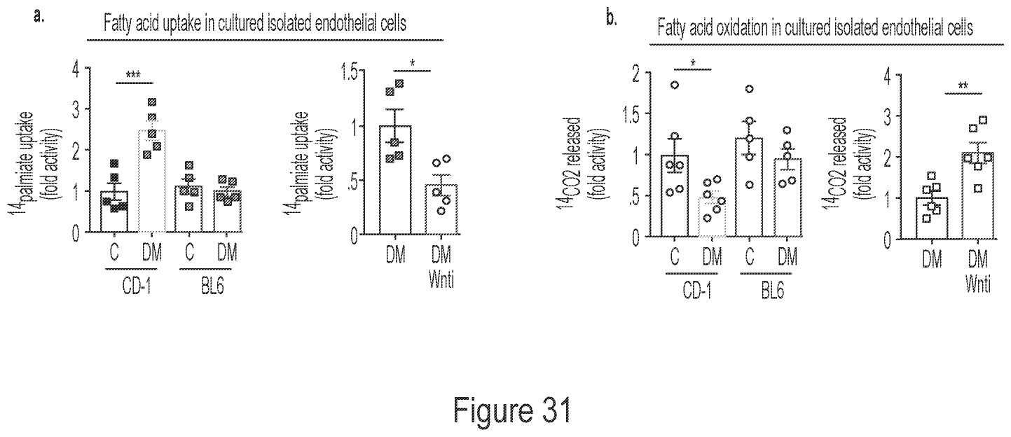

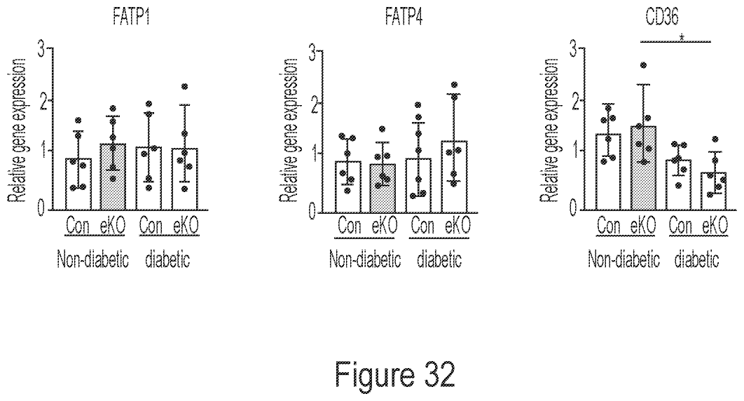

It is increasingly recognized that defects in central metabolism contribute to kidney fibrosis^{32,41}. Defective fatty acid (FA) metabolism in ECs leads to EndMT events⁴². To investigate whether FA metabolism was deranged in the model, radiolabeled [¹⁴C]palmitate uptake experiments in isolated ECs from mouse kidneys were performed. the inventors observed that FA uptake was higher in isolated ECs from diabetic kidneys of the more fibrotic strain (diabetic CD-1) when compared to kidney ECs from the less fibrotic strain (diabetic C57BL/6). Administration of the Wnt inhibitor suppressed FA uptake in ECs from CD-1 mice (FIG. 31A). Kidney ECs from both diabetic GR^{ECKO} and DKO mice displayed higher FA uptake when compared to those of the diabetic control littermates (FIG. 6A). FA oxidation (FAO) was also assessed by measuring the [¹⁴CO₂]release from radiolabeled [¹⁴C]palmitate in cultured ECs isolated from kidneys. FAO was diminished in the isolated kidney ECs of diabetic CD-1 mice and Wnt inhibition was able to restore the level of FAO (FIG. 31B). Cultured kidney ECs from diabetic GR^{ECKO} and diabetic DKO mice showed a diminished level of FAO when compared to their diabetic control littermates (FIG. 6B). In addition, gene expression of fatty acid transporter proteins 1 and 4 (FATP1 and FATP4) were unaltered in both nondiabetic and diabetic ECs isolated from kidneys of control and GR^{ECKO} mice; however, the gene expression level of CD36, another fatty acid transporter, was lower in diabetic mice of both genotypes when compared to non-diabetic controls (FIG. 32).

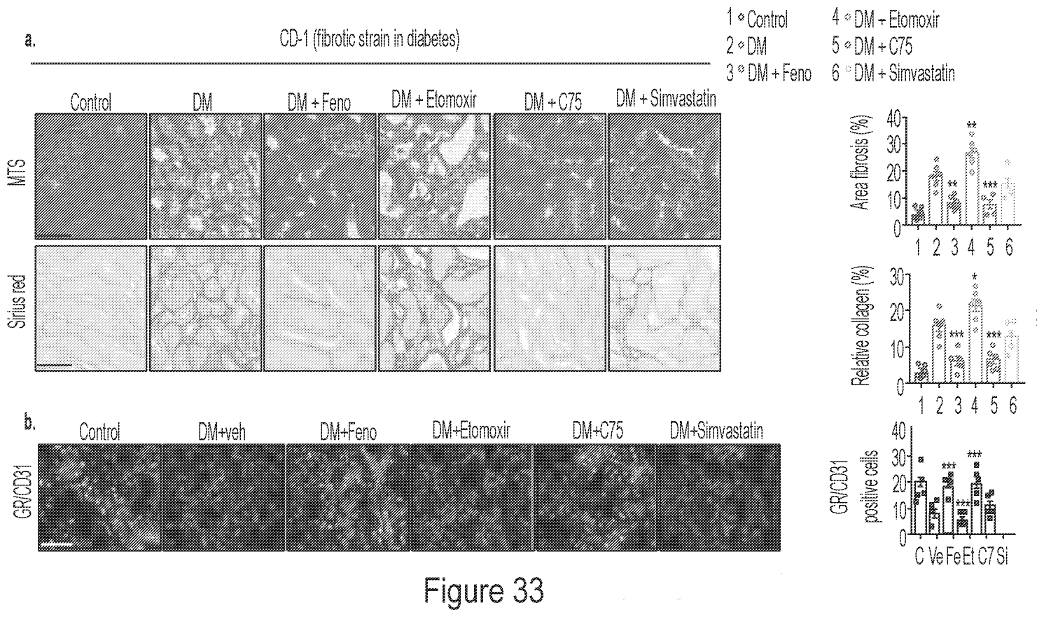

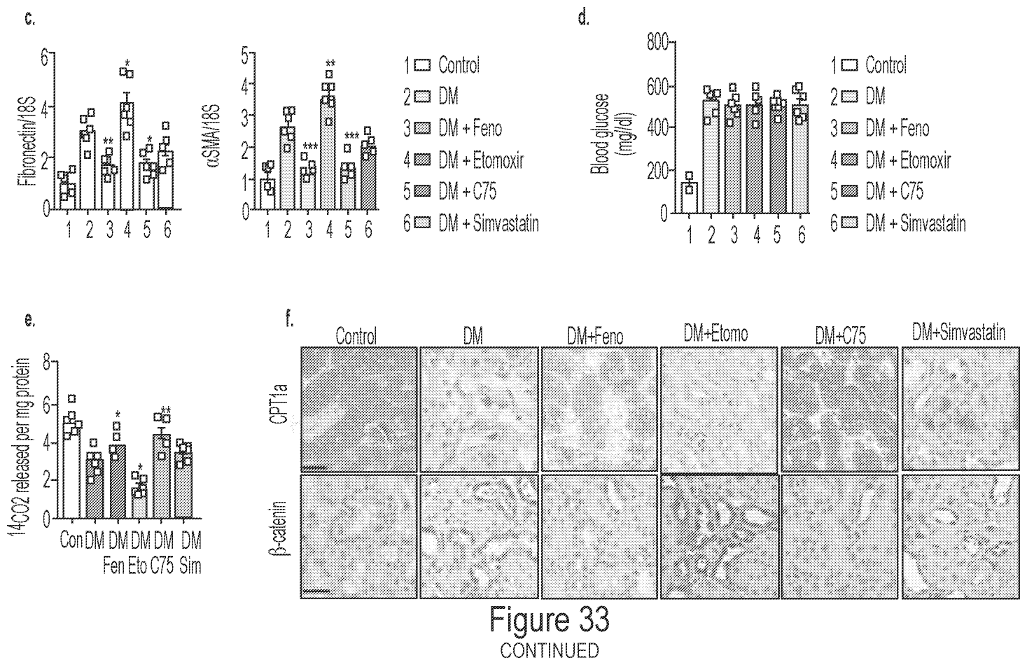

In the next set of experiments, diabetic CD-1 mice were treated with the FA synthase inhibitor C75, the FAO inhibitor etomoxir, the PPARα agonist fenofibrate, or the cholesterol-lowering drug simvastatin for 4 weeks. Fenofibrate and C75 ameliorated the fibrogenic phenotype, whereas etomoxir exacerbated the fibrosis. Simvastatin treatment did not cause significant suppression in the level of fibrosis. (FIG. 33A). Fenofibrate and C75 restored the level of GR protein in CD31 positive cells, whereas etomoxir suppressed it and simvastatin did not show any substantial effect (FIG. 33B). Fenofibrate and C75 downregulated fibronectin and αSMA mRNA levels, while etomoxir upregulated this mRNA and simvastatin did not cause any significant change in the gene expression level of fibronectin or αSMA in the diabetic kidneys (FIG. 33C). These FA modulators did not cause any significant differences in the blood glucose levels (FIG. 33D). Etomoxir treatment caused significant suppression of FAO, as measured by [¹⁴CO₂]release, and CPT1a level, and induced the expression of β-catenin whereas C75 and fenofibrate increased the level of FAO, increased the expression of CPT1a and suppressed the level of β-catenin in diabetic CD-1 mice (FIGS. 33E-33F).

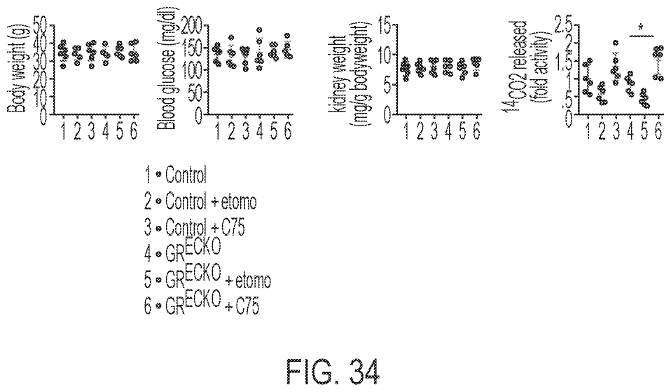

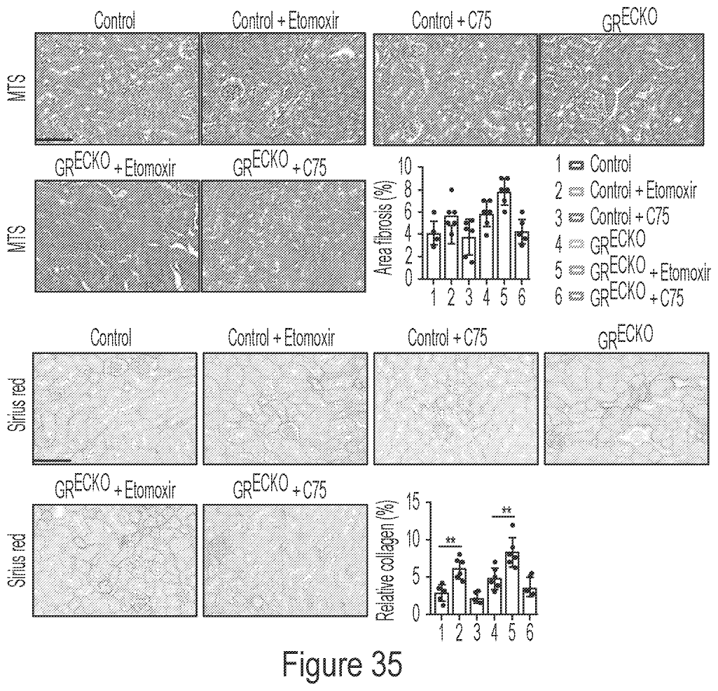

Etomoxir and C75 were also tested in non-diabetic and diabetic control littermates and GR^{ECKO} mice (FIGS. 6C-6D; FIG. 34; FIG. 35). There were no significant differences in body weight, blood glucose or kidney weight in non-diabetic or diabetic control littermates and GR^{ECKO} mice after treatment with etomoxir or C75 (FIG. 6C; FIG. 34). Data from kidney ECs revealed that etomoxir caused significant suppression of FAO, and C75 restored FAO in non-diabetic control littermates (FIG. 34). However, in kidney ECs from diabetic GR^{ECKO} mice, etomoxir caused significant suppression of FAO which C75 was unable to rescue (FIG. 6c). Etomoxir treatment accelerated the renal fibrogenic phenotype, suppressed the CPT1a level and increased the expression level of β-catenin in the kidneys of diabetic control and diabetic GR^{ECKO} mice. C75 treatment clearly abolished the renal fibrogenic phenotype, restored CPT1a and completely diminished the level of β-catenin in the kidneys of diabetic control mice. These effects were also observed in the GR^{ECKO} mice, though to a lesser extent. (FIG. 6D). Etomoxir slightly increased the level of renal fibrosis in nondiabetic control and GR^{ECKO} mice which could be mitigated by C75 (FIG. 35). Etomoxir decreased expression of CPT1a in diabetic control mice while also increasing expression of β-catenin; C75 was able to substantially reverse both of these effects. A similar pattern was observed in diabetic GR^{ECKO} mice, though again, to a lesser extent (FIG. 6E).

### Example 8. GR Loss-Linked EndMT Disrupts Central Metabolism and Induces Mesenchymal Transformation in Tubular Epithelial Cells

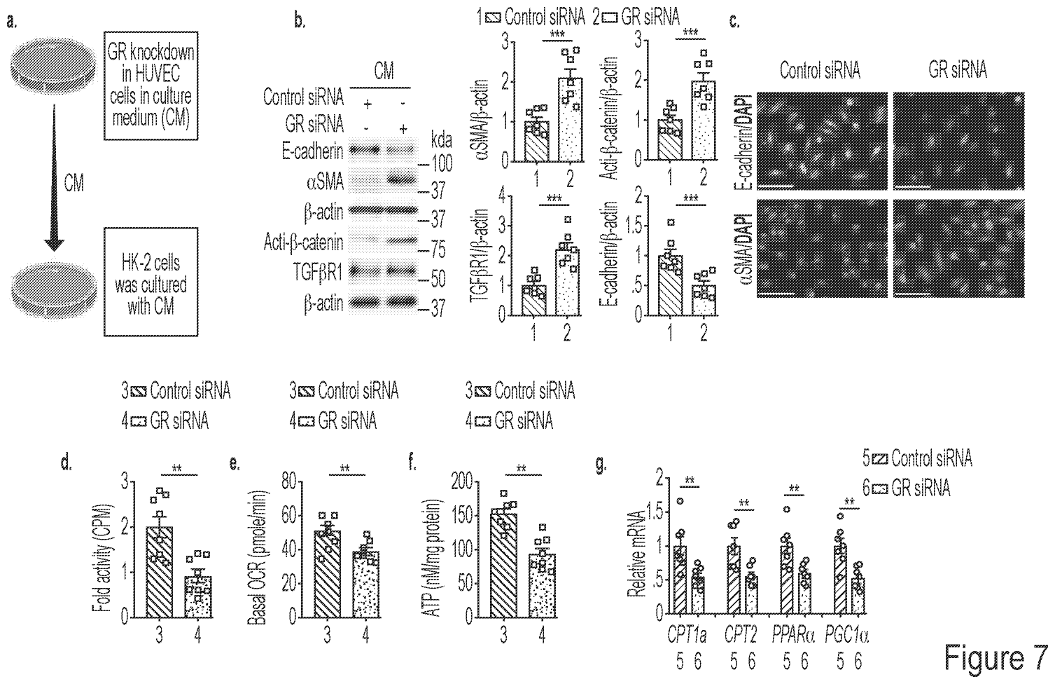

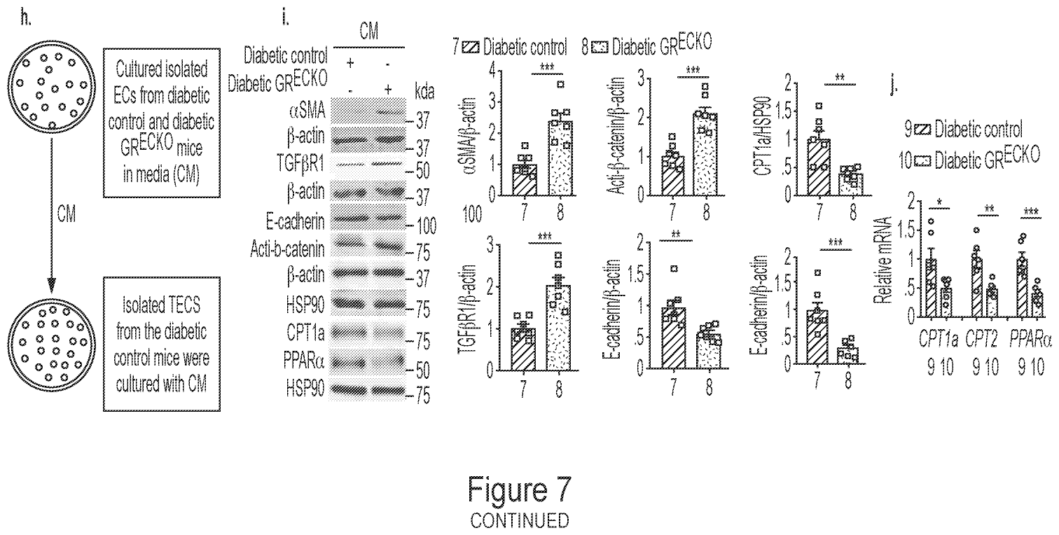

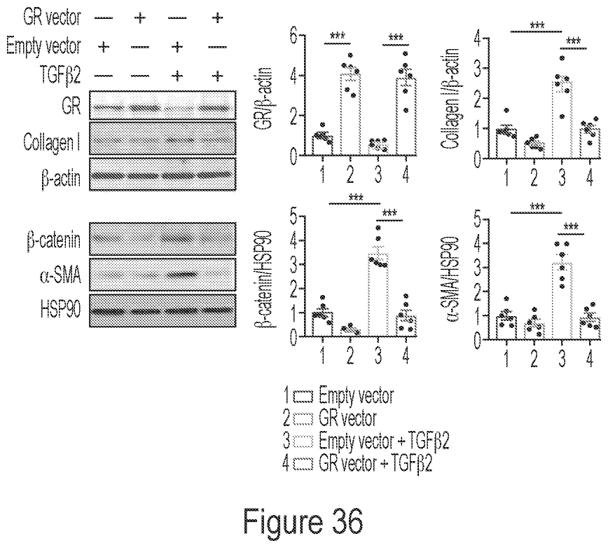

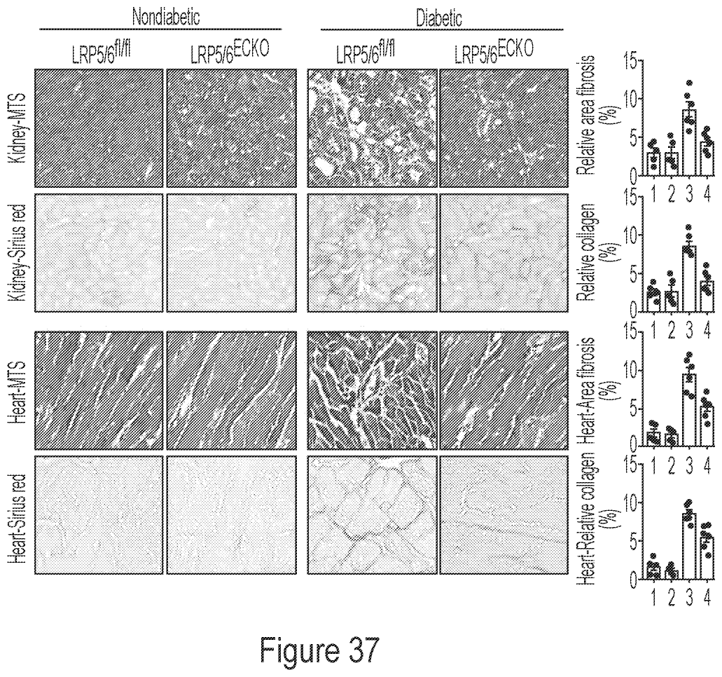

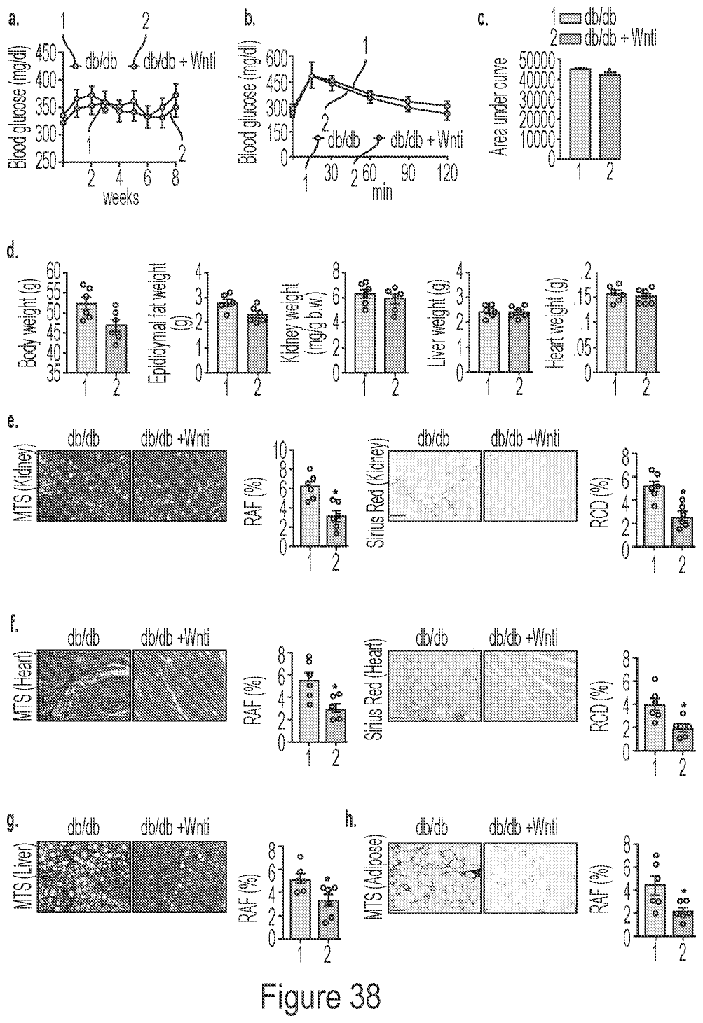

This in vivo data suggests that GR^{ECKO} mice exhibit enhanced EndMT and EMT in their diabetic kidneys. Overexpression of GR suppressed the TGFβ2-stimulated increase in αSMA, collagen I and β-catenin expression in HUVECs (FIG. 36). To test whether endothelial GR deficiency affects mesenchymal programs and causes defects in central metabolism in neighboring epithelial cells, the inventors analyzed the effects of conditioned media from GR knockdown HUVECs on the mesenchymal phenotype of HK-2 cells (FIG. 7A). Conditioned media (CM) from GR siRNA-transfected HUVECs decreased E-cadherin protein levels and increased αSMA, TGFβR1 and β-catenin protein levels in HK-2 cells when compared to media from scrambled siRNA-transfected HUVECs (FIGS. 7B-7C). CM treatment from GR siRNA-transfected HUVECs caused a significant reduction in the level of FAO, oxygen consumption rate and cellular ATP level in HK-2 cells (FIGS. 7D-7F). TECs incubated with CM from GR siRNA-treated HUVECs exhibited significantly down-regulated mRNA expression of the FAO-responsive genes Cpt1a, Cpt2, Ppara, and Pgc1α compared to TECs incubated with CM from control siRNA-treated HUVECs (FIG. 7G).

To confirm these in vitro results, primary ECs from diabetic control and diabetic GR^{ECKO} mice were isolated to analyze the contribution of GR-deficient ECs on mesenchymal activation in tubular epithelial cells (TECs). CM from isolated cultured ECs from the kidneys of diabetic GR^{ECKO} and diabetic control littermates was transferred to cultured TECs from diabetic control mice (FIG. 7H). This CM treatment from GR-deficient cells caused significant suppression of E-cadherin, CPT1a and PPARα and induced αSMA, TGFβR1 and active β-catenin protein levels in TECs (FIG. 7I). Furthermore, CM from ECs from diabetic GR^{ECKO} mice significantly downregulated mRNA expression of the FAO-responsive genes Cpt1a, Cpt2 and Ppara (FIG. 7J).

The Examples presented above demonstrate the crucial role of endothelial GR in the regulation of fibrogenic processes in a mouse model of diabetic kidney disease. The results demonstrate that endothelial GR regulates the mesenchymal trans differentiation process by influencing FA metabolism and control over canonical Wnt signaling in the kidneys of diabetic mice. It is clear from these data that GR loss is one of the catalysts of renal fibrosis in diabetes that leads to disruption of cytokine and chemokine homeostasis by up regulating canonical Wnt signaling. Ultimately, these processes alter the metabolic switch in favor of defective FA metabolism and associated mesenchymal activation in TECs.

Metabolic reprogramming in ECs is a critical event in the development of myofibroblast formation, proliferation and fibrosis in diabetic kidneys^{12,17,41,43,44}. The data suggests GR deficiency is a critical step in the metabolic reprogramming of kidney ECs. While bilateral adrenalectomy suppressed corticosterone significantly in all mice studied, this global loss of systemic steroid signaling was not sufficient to alter the course of fibrosis in diabetic mice, suggesting that the tissue-specific effects of targeted loss of GR in the endothelium supersede the systemic effects; this phenomenon will require further study. The altered cytokine levels in the plasma of GR^{ECKO} mice include elevated levels of pro-inflammatory cytokines (IL-1β, IL-6, and IL-17) and the anti-inflammatory cytokine IL-10. The role of IL-10 has not been fully investigated in renal fibrosis in diabetic kidney disease so far. However, these data demonstrate that IL-6 is a key inflammatory cytokine which is elevated in states of endothelial GR suppression. The neutralization of IL-6 in diabetic mice completely reversed the renal fibrotic phenotype, suggesting a critical profibrotic role of IL-6 in diabetes.

Recently, it was demonstrated that loss of endothelial GR results in up-regulation of canonical Wnt signaling²⁷. It is accepted that GR performs its anti-inflammatory actions by targeting the NF-kB signaling pathway⁴⁵. However, GR targets also canonical Wnt signaling in ECs, which is independent of its classic target, NF-kB^{27,45}. Inhibition of Wnt signaling in ECs may prove to be a valuable therapeutic opportunity for combatting diabetic kidney disease. The Wnt pathway is known to be an important contributor to renal fibrosis and activated canonical Wnt signaling contributes to the disruption of cytokine and chemokine homeostasis^{39,46-48}. These data demonstrate that higher levels of GR-deficient-canonical Wnt signaling are associated with the induction of mesenchymal and fibrogenic markers.

To further test the therapeutic potential of Wnt inhibition, the inventors used the small molecule -LGK974. Wnt inhibition clearly suppressed canonical Wnt signaling, substantially improved the fibrogenic phenotype in the mouse model of diabetic kidney disease and restored the endothelial GR level. These data suggest that tonic repression of canonical Wnt signaling in ECs is one mechanism by which GR performs its anti-fibrotic action. Notably, this effect was less evident in GR^{ECKO} mice, possibly since Wnt inhibition was able to suppress EMT processes in other cell types (TECs) while it was unable to mitigate EndMT processes. Cumulatively, these data suggest that endothelial GR is a key anti-EndMT molecule.

Research investigating lipid metabolism in kidney cells is limited^{49,50} but gaining importance. Defects in central metabolism contribute to diabetic kidney disease^{32,41} and there are a few reports showing that altered cytokine levels can affect renal lipid metabolism in diabetic kidney disease^{51,52}. Clinical observations indicate a potential association between lipid levels and kidney disease⁵³, and lipid control appears to be important in the prevention and treatment of diabetic kidney disease^{49,50}. Here, the inventors aimed to dissect the contribution of lipid metabolism in ECs to the regulation of diabetic kidney disease.

DKO mice show worsened atherosclerosis, compared to Apoe^{-/-} mice, which is not explained by differences in plasma lipid levels²⁵. This observation was the catalyst which led to the evaluation of the diabetic phenotype in these animals. It is clear from the data that hypercholesterolemia worsened the severity of renal fibrosis in GR^{ECKO} mice, suggesting that hypercholesterolemia affects EC metabolism and contributes to renal fibrosis. However, similar to available clinical data, the cholesterol-lowering drug simvastatin did not ameliorate the severity of renal fibrosis in this mouse model of diabetic kidney disease. Interestingly, fibrates are a class of drugs that treat hypertriglyceridemia with residual elevation of non-HDL cholesterol. However, the role of fibrates in patients with diabetic kidney disease has yet to be determined^{50,63}.

The inventors assessed the contribution of endothelial GR loss-linked Wnt activation and its association with defects in FA metabolism. Disruption of endothelial FA metabolism contributes to activation of EndMT in diabetic kidneys^{8,31,64}. FAO activation caused remarkable suppression of fibrosis by restoring the endothelial GR level in diabetic mice. In contrast, FAO inhibition caused acceleration of fibrosis by diminishing the level of endothelial GR in diabetic control mice, suggesting that endothelial GR is a critical protein for the action of FAO modulators. These data clearly suggest that the antifibrotic effect of the FAO activator C75 is dependent on endothelial GR, in turn suggesting that ECs are required for the anti-fibrotic action of this drug.

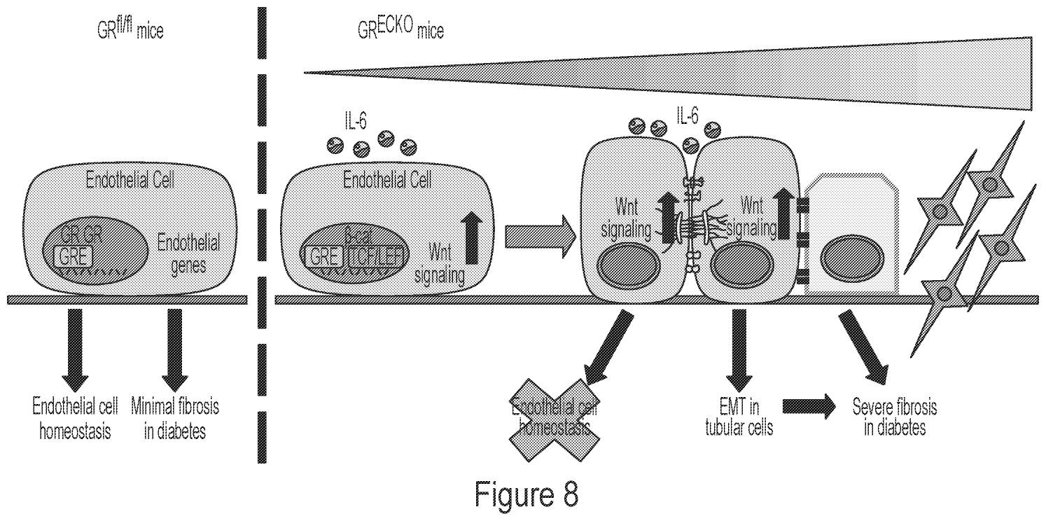

When conditioned media from GR-deplete ECs from diabetic GR^{ECKO} mice were transferred to cultured TECs from diabetic control kidneys, the inventors observed induction of mesenchymal markers, activation of TGFβ and canonical Wnt signaling and concomitant suppression of epithelial cell markers. These findings suggest that EndMT leads to the activation of EMT processes in diabetes. GR-deplete cells have higher levels of TGFβ-smad3 and canonical Wnt signaling, which are associated with disrupted levels of plasma cytokines and suppressed FAO. The cumulative effects of these metabolic changes result in activation of mesenchymal transformation in ECs which appears to exert paracrine effects on neighboring TECs. The functional importance of GR protein in EC homeostasis is depicted graphically (FIG. 8).

GR agonists like dexamethasone activate GR signaling in all cell types; however, in diabetes, dexamethasone intervention is not preferred due to the severe and predictable exacerbation of hyperglycemia. These data highlight the regulatory role of GR in ECs.

In conclusion, these findings highlight the regulatory role of GR on EndMT in diabetic kidneys, mediated by control over canonical Wnt signaling and linked defective FA metabolism. This study provides insight into the biology of GR and its critical role in renal fibrosis and diabetes.

Below are the methods used in the Examples described above.

Reagents and antibodies. Rabbit polyclonal anti-GR (SAB4501309), mouse monoclonal anti-αSMA (Cat:A5228) and mouse monoclonal anti-β-actin (AC-74) (A2228) antibodies were from Sigma (St Louis, MO). Anti-TGFβR1 (ab31013), PPARα (ab215270), mouse monoclonal anti-vimentin (RV202) (ab8978), rabbit polyclonal anti-αSMA (ACTA2) (ab5694), anti-HIF1α (ab516008) and goat polyclonal anti-Snail1 (ab53519) antibodies were purchased from Abcam (Cambridge, UK). Mouse anti-β-catenin antibody (610154) was purchased from BD Biosciences. Carnitine palmitoyltransferase 1a (CPT1a) (12252), rabbit polyclonal anti-E-cadherin antibody (24E10) (3195) and rabbit non-phospho (active) β-Catenin (8814) antibodies were purchased from Cell Signaling Technology (Danvers, MA, USA). Anti-HSP90 was purchased from BD Biosciences (610419). In vivo mouse IL-6 IgG neutralization antibody and control IgG antibodies were purchased from Bio X Cell. Fluorescence-, Alexa fluor 647-, and rhodamine-conjugated secondary antibodies were obtained from Jackson ImmunoResearch (West Grove, PA). TGFβ2, IL-1p and recombinant TNFα and TGFβ neutralizing antibodies were purchased from PeproTech (Rocky Hill, NJ). Etomoxir, C75 and Wnt inhibitor (LGK974) were purchased from Cayman Chemical (Ann Arbor MI).

Animal Experimentation. All experiments were performed according to a protocol approved by the Institutional Animal Care and Use Committee at the Yale University School of Medicine and were in accordance with the National Institute of Health (NIH) Guidelines for the Care of Laboratory Animals Mice were housed at an ambient temperature of 68-79° F. with a humidity that ranged between 30%-70%. They were exposed to 12-h light-dark cycles Mice lacking the endothelial glucocorticoid receptor (GR) (known as GR^{ECKO}) and those lacking this receptor on the Apoe null background (DKO) were generated as previously described²⁵; these mice were on the C57BL/6 background. Diabetes was induced in 10-week-old male mice with five consecutive intraperitoneal (IP) doses of streptozotocin (STZ) 50 mg/kg in 10 mmol/L citrate buffer (pH 4.5). Wnt inhibitor (LGK974) was provided to 16 week-STZ-treated diabetic mice using a dose of 5 mg/kg at a frequency of six doses per week for 8 weeks¹⁰. Etomoxir (20 mg/kg) and C75 (15 mg/kg) were dosed (IP) three times per week for 3 weeks in GR^{ECKO} and control littermates.

A single IP dose of 200 mg/kg STZ was used to induce diabetes in CD-1 mice.

In one experiment, male mice were randomized to one of 4 groups sixteen weeks after induction of diabetes: (i) untreated, (ii) fenofibrate (100 mg/kg), (iii) simvastatin (40 mg/kg), or (iv) Wnt inhibitor (LGK974; 5 mg/kg). In each case, mice were treated for 4 weeks and compared to untreated diabetic CD-1 mice. In another experiment, male diabetic CD-1 mice were randomized into one of 3 groups: (i) untreated (vehicle), (ii) etomoxir (20 mg/kg) or (iii) C75 (15 mg/kg); in each case mice were treated (IP) three times/week for a total 4 weeks.

IL-6 IgG and control IgG were injected IP three times/week for a total four weeks at a dose of 3 mg/kg in both non-diabetic and diabetic mice. All mice had free access to food and water during experiments. Blood was obtained by retro-orbital bleed during experiments. Blood glucose was measured by glucose-strips. Urine albumin levels were assayed using a Mouse Albumin ELISA Kit (Exocell, Philadelphia, PA).

Tissues and blood were harvested at the time of sacrifice. Some kidneys were minced and stored at −80° C. for gene expression and protein analysis. Other kidneys were placed immediately in optimal cutting temperature (OCT) compound for frozen sections or 4% paraformaldehyde for histologic staining.

Bilateral adrenalectomy. Sixteen week-STZ-injected (24-week old) diabetic mice and nondiabetic CD-1 and C57BL/6 mice were used for adrenalectomy and sham operations. Animals were handled daily during the last week before experimentation to reduce stress responses. Buprenorphine was used as an analgesic. The first dose was administered 30 minutes before surgery and then every 12 h for 72 h, at a dose of 0.05 mg/kg subcutaneously. For the first 24 hours after surgery, mice were given drinking water containing 0.9% saline to counter the effect of mineralocorticoid removal. Adrenalectomized animals were sacrificed 4 weeks after surgery. Blood was withdrawn in the morning between 9:00 and 10:00 AM. Corticosterone levels were measured using an ELISA from Cayman Chemical (Ann Arbor MI).

Mouse model of unilateral ureteral obstruction (UUO). UUO surgical procedure was performed as previously described¹⁹. Briefly, mice were anesthetized with isoflurane (3%-5% for induction and 1%-3% for maintenance). Mice were shaved on the left side of the abdomen, a vertical incision was made through the skin with a scalpel, and the skin was retracted. A second incision was made through the peritoneum to expose the kidney. The left ureter was ligated twice 15 mm below the renal pelvis with surgical silk, and the ureter was then severed between the 2 ligatures. Then, the ligated kidney was placed gently back into its correct anatomical position, and sterile saline was added to replenish loss of fluid. The incisions were sutured and mice were individually caged. Buprenorphine was used as an analgesic. The first dose was administered 30 minutes before surgery and then every 12 h for 72 h, at a dose of 0.05 mg/kg subcutaneously. Mice were sacrificed and kidney and blood samples were harvested after perfusion with PBS at 10 days after UUO. Contralateral kidneys were used as a nonfibrotic control for all experiments using this model.

Blood pressure measurement. Measurements were taken using the tail cuff method according to the manufacturer's instructions. Briefly, mice were trained for 5 days before measurement of blood pressure. After mice were placed in the restraint platform, which was maintained at 33-34° C., the tail was placed through the optical sensor and the cuff compressed. The instrument automatically measured the blood pressure and repeated this 10 times. Data are presented as the average of 10 measurement cycles.

Lipid Analysis. Mice were fasted for 12-15 hours and blood was collected by retro-orbital venous puncture. Whole blood was spun down and plasma stored at −80° C. Total cholesterol and triglyceride levels were measured enzymatically by kits from Wako and Sigma, respectively, according to the manufacturer's instructions.

Morphological Evaluation. A point-counting method was utilized to evaluate the relative area of the mesangial matrix. The inventors analyzed PAS-stained glomeruli from each mouse using a digital microscope screen grid containing 540 (27×20) points. Masson's trichrome-stained images were evaluated by ImageJ software, and the fibrotic areas were estimated.

Sirius red staining. Deparaffinized sections were incubated with picrosirius red solution for 1 hour at room temperature. The slides were washed twice with acetic acid solution for 30 seconds per wash. The slides were then dehydrated in absolute alcohol three times, cleared in xylene, and mounted with a synthetic resin. Sirius red staining was analyzed using ImageJ software, and fibrotic areas were quantified.

Immunohistochemistry. Paraffin-embedded kidney sections (5 μm thick) were deparaffinized and rehydrated (2 min in xylene, four times; 1 min in 100% ethanol, twice; 1 min in 95% ethanol; 45 s in 70% ethanol; and 1 min in distilled water), and the antigen was retrieved in a 10 mM citrate buffer pH 6 at 98° C. for 60 min. To block the endogenous peroxidase, all sections were incubated in 0.3% hydrogen peroxide for 10 min. The immunohistochemistry was performed using a Vectastain ABC Kit (Vector Laboratories, Burlingame, CA). Mouse anti-β-catenin antibody (1:100) and CPT1a (Abnova; H00001374-DO1P; 1:100) antibodies were used. In the negative controls, the primary antibody was omitted and replaced with the blocking solution.

Immunofluorescence. Frozen kidney sections (5 μm) were used for immunofluorescence; double positive labeling with CD31/αSMA (1:100/1:500), CD31/TGFβR1 (1:100/1:500) and E-cadherin/αSMA (1:500/1:500) was measured. Briefly, frozen sections were dried and placed in acetone for 10 min at −30° C. Once the sections were dried, they were washed twice in phosphate-buffered saline (PBS) for 5 min and then blocked in 2% bovine serum albumin/PBS for 30 min at room temperature. Thereafter, the sections were incubated in primary antibody (1:100) for 1 h and washed in PBS (5 min) three times. Next, the sections were incubated with the secondary antibodies for 30 min, washed with PBS three times (5 min each), and mounted with mounting medium with DAPI (Vector Laboratories, Burlingame, CA). The immuno-labeled sections were analyzed by fluorescence microscopy. For each mouse, original magnification of ×400 pictures were obtained from six different areas, and quantification was performed.

EndMT and EMT detection. Frozen sections (5 μm) were used for the detection of EndMT and EMT. Cells undergoing EndMT were detected by double-positive labeling for CD31 (1:100) and αSMA (1:500) and/or TGFβR1 (1:500). Cells undergoing EMT were detected by double-positive labeling for E-cadherin (1:500) and αSMA (1:500). Sections were analyzed and quantified by fluorescence microscopy.

Isolation of endothelial cells. Endothelial cells from the kidneys of non-diabetic and diabetic mice were isolated using a standardized kit (Miltenyl Biotech, USA) by following the manufacturer's instructions. Briefly, kidneys were isolated and minced into small pieces. Using a series of enzymatic reactions by treating the tissue with trypsin and Collagenase type I solution, a single cell suspension was created. The pellet was dissolved with CD31 magnetic beads and the CD31-labelled cells were separated on a magnetic separator. The cells were further purified on a column. Cell number was counted by hemocytometer and cells were plated on 0.1% gelatin coated Petri dishes. Cell purity was measured by flow cytometry (BD FACSDiva) using PE-conjugated CD31 (BDB553373) (1:100) and FITC-conjugated CD45 (BDB553079) (1:100), both from Becton Dickinson (USA).

Isolation of kidney TECs. After sacrifice kidneys from diabetic GR^{ECKO} and control littermate were excised and perfused with (10 mL) followed by collagenase type II digestion (2 mg/mL). After digestion, the cortical region of kidneys was used for further processing. the cortical region of kidneys was minced and further digested in collagenase buffer for an additional 5 minutes at 37° C. with rotation to release cells. Digested tissue and cell suspension were passed through a 70-μm cell strainer, centrifuged at 50 g for 5 min, and washed in PBS for 2 rounds to collect TECs. Isolated TECs were seeded onto collagen-coated Petri dishes and cultured in renal epithelial cell medium (C-26130, PromoCell) supplemented with growth factors for TEC growth.

Cellular bioenergetic analysis. FAO-associated oxygen consumption rate (OCR) was studied using extracellular flux analysis (Seahorse XFe96, Agilent Technologies). On the assay day, substrate-limited medium was replaced with Krebs-Henseleit buffer assay medium supplemented with 0.2% carnitine for 1 h at 37° C. without CO₂. Finally, just before starting the assay, BSA or 200 mM palmitate-BSA FAO substrate was added. After the assay, protein was extracted from wells with 0.1% NP-40-PBS solution and quantified with a bicinchoninic acid protein assay (Thermo Fisher Scientific) for data normalization. OCR was determined by normalizing the measurements to cell counts by quantifying the Hoechst staining in each well⁶⁵.

ATP measurement. ATP content was determined using the ATP Colorimetric Assay kit (Biovision), following the manufacturer's instructions.

RNA isolation and qPCR. Total RNA was isolated using standard Trizol protocol. RNA was reverse transcribed using the iScript cDNA Synthesis kit (Bio-Rad) and qPCR was performed on a Bio-Rad C1000 Touch thermal cycler using the resultant cDNA, as well as qPCR Master mix and gene specific primers. The list of mouse primers used is in Table 1. Results were quantified using the delta-delta-cycle threshold (Ct) method (ΔΔCt). All experiments were performed in triplicate and 18S was utilized as an internal control.

TABLE 1
Sequences of mouse primers

| | | SEQ | | SEQ |
|---|---|---|---|---|
| | | ID | | ID |
| Gene | Forward primer | NO: | Reverse primer | NO: |
| CD31 | 5′-AGGCTTGCATAGA | 1 | 5′-TTCTTGGTTTCCAG | 14 |
| | GCTCCAG | | CTATGG | |
| aSMA | 5′-CTGACAGAGGCAC | 2 | 5′-GAAATAGCCAAGCT | 15 |
| | CACTGAA | | CAG | |
| Axin2 | 5′-AACCTATGCCCGT | 3 | 5′-GAGTGTAAAGACTT | 16 |
| | TTCCTCTA | | GGTCCACC | |
| Tcf | 5′-GGTGGCCGAATGC | 4 | 5′-TTTGCCTGTTCTTC | 17 |
| | ACATTGAAAGA | | CCTGGACA | |
| FN | 5′-CGAGGTGACAGAG | 5 | 5′-CTGGAGTCAAGCCA | 18 |
| | ACCACAA | | GACACA | |
| IL-1β | 5′-CCAAGCAACGACA | 6 | 5′-GTTGAAGACAAACC | 19 |
| | AAATACC | | GTTTTTCC | |
| IL-6 | 5′-TCTGAAGGACTCT | 7 | 5′-GATGGATGCTACCA | 20 |
| | GGCTTTG | | AACTGGA | |
| IL-10 | 5′-CACAAAGCAGCCT | 8 | 5′-AGAGCAGGCAGCAT | 21 |
| | TGCAGAA | | AGCAGTG | |
| IL-17 | 5′GCTCCAGAAGGCCC | 9 | 5′CTTTCCCTCCGCATT | 22 |
| | TCAGA | | GACA | |
| CCL4 | 5′CTCAGCCCTGATGC | 10 | 5′AGAGGGGCAGGAAAT | 23 |
| | TTCTCAC | | CTGAAC | |
| CCL5 | 5′GCTGCTTTGCCTAC | 11 | 5′TCGAGTGACAAACAC | 24 |
| | CTCTCC | | GACTGC | |
| Eotaxin | 5′-AGCTAGTCGGGAG | 12 | 5′-AAGGAAGTGACCGT | 25 |
| | AGCCTAC | | GAGCAG | |
| 18S | 5′TTCCGATAACGAAC | 13 | 5′GGCTGAACGCCACTT | 26 |
| | GAGACTCT | | GTC | |

Western blot. Protein lysates were boiled in sodium dodecyl sulfate (SDS) sample buffer at 94′C for 5 min. After centrifugation at 17,000×g for 10 min at 4° C., the supernatant was separated on 6%-12% SDS polyacrylamide gels, and blotted onto PVDF membranes (Immobilon, Bedford, MA) via the semidry method. After blocking with TB S (Tris buffered saline containing 0.05% Tween 20) containing 5% bovine serum albumin (BSA), membranes were incubated with each primary antibody (GR: 1:1000; Anti-TGFβR1: 1:500; anti-αSMA: 1:500; anti-vimentin: 1:2000, anti-β-catenin: 1:500, anti-β-actin 1:10,000), in TBS containing 5% BSA at 4° C. overnight. Protein bands were visualized using the Odyssey Infrared Imaging System (LI-COR Biotechnology), and enhanced chemiluminescence (ECL) detection system (Pierce Biotechnology, Rockland IL) using ImageQuant LAS 400 (GE Healthcare Life Sciences, Uppsala, Sweden). Densitometry was performed using ImageJ software (NIH).

In vitro experiments and siRNA transfection. HUVECs were used at passage 4-8 and cultured in Endothelial Basal Medium-2 media with growth factors and 10% serum. Human GR-specific siRNA (Invitrogen) was used at a concentration of 100 nM for 48 h to effectively knock down GR. Cells were treated with or without TGFβ2 (10 ng/ml) for 48 h and harvested for western blot analysis. Some transfected cells were treated with fenofibrate (1 μM) and etomoxir (40 μM) for 48 h. In a second set of experiments Human HK-2 cells were cultured in DMEM and Keratinocyte-SFM (1×) medium (Life Technologies Green Island NY). When the cells reached 70% confluence, conditioned media from control siRNA and GR siRNA-transfected HUVECs was added to the HK-2 cell culture.

Fatty acid uptake. Cultured isolated kidney endothelial cells were incubated with medium containing 2 μCi [¹⁴C]-palmitate. [¹⁴C]-palmitate uptake was measured by liquid scintillation counting.

Fatty Acid Oxidation. Cultured isolated kidney endothelial cells were incubated with medium containing 0.75 mmol/L palmitate (conjugated to 2% free fatty acid-free BSA/[¹⁴C]palmitate at 2 μCi/mL) for 2 h. One mL of the culture medium was transferred to a sealable tube, the cap of which housed a Whatman filter paper disc. ¹⁴CO₂ trapped in the media was then released by acidification of media using 60% perchloric acid. Radioactivity that had become adsorbed onto the filter discs was then quantified by liquid scintillation counting.

Statistical analysis. All values are expressed as means±SEM and analyzed using the statistical package for the GraphPad Prism 7 (GraphPad Software, Inc., La Jolla, CA). One-way ANOVA, followed by Tukey's test and Two-way ANOVA (FIGS. 1 and 2) were employed to analyze significance when comparing multiple independent groups. The post hoc tests were run only if F achieved P<0.05 and there was no significant variance in homogeneity. In each experiment, N represents the number of separate experiments (in vitro) and the number of mice (in vivo). Technical replicates were used to ensure the reliability of single values. Data analyses were blinded. The data were considered statistically significant at p<0.05.

### REFERENCES

1 Reidy, K., Kang, H. M., Hostetter, T. & Susztak, K. Molecular mechanisms of diabetic kidney disease. J Clin Invest 124, 2333-2340, doi:10.1172/JCI72271 (2014).
2 Badal, S. S. & Danesh, F. R. New insights into molecular mechanisms of diabetic kidney disease. Am J Kidney Dis 63, S63-83, doi:10.1053/j.ajkd.2013.10.047 (2014).
3 Breyer, M. D. & Susztak, K. The next generation of therapeutics for chronic kidney disease. Nat Rev Drug Discov 15, 568-588, doi:10.1038/nrd.2016.67 (2016).
4 Humphreys, B. D. et al. Fate tracing reveals the pericyte and not epithelial origin of myofibroblasts in kidney fibrosis. The American journal of pathology 176, 85-97, doi:10.2353/ajpath.2010.090517 (2010).
5 Kuppe, C. et al. Decoding myofibroblast origins in human kidney fibrosis. Nature, doi:10.1038/s41586-020-2941-1 (2020).
6 LeBleu, V. S. et al. Origin and function of myofibroblasts in kidney fibrosis. Nat Med 19, 1047-1053, doi:10.1038/nm.3218 (2013).
7 Srivastava, S. P., Koya, D. & Kanasaki, K. MicroRNAs in Kidney Fibrosis and Diabetic Nephropathy: Roles on EMT and EndMT. Biomed Res Int 2013, 125469, doi:10.1155/2013/125469 (2013).
8 Srivastava, S. P., Hedayat, F. A., Kanasaki, K. & Goodwin, J. E. microRNA Crosstalk Influences Epithelial-to-Mesenchymal, Endothelial-to-Mesenchymal, and Macrophage-to-Mesenchymal Transitions in the Kidney. Front Pharmacol 10, 904, doi:10.3389/fphar.2019.00904 (2019).
9 Medici, D. Endothelial-Mesenchymal Transition in Regenerative Medicine. Stem Cells Int 2016, 6962801, doi:10.1155/2016/6962801 (2016).
10 Eelen, G. et al. Endothelial Cell Metabolism. Physiol Rev 98, 3-58, doi:10.1152/physrev.00001.2017 (2018).
11 Theodorou, K. & Boon, R. A. Endothelial Cell Metabolism in Atherosclerosis. Front Cell Dev Biol 6, 82, doi:10.3389/fcell.2018.00082 (2018).
12 Zhou, H. L. et al. Metabolic reprogramming by the S-nitroso-CoA reductase system protects against kidney injury. Nature 565, 96-100, doi:10.1038/s41586-018-0749-z (2019).
13 Cantelmo, A. R. et al. Inhibition of the Glycolytic Activator PFKFB3 in Endothelium Induces Tumor Vessel Normalization, Impairs Metastasis, and Improves Chemotherapy. Cancer Cell 30, 968-985, doi:10.1016/j.ccell.2016.10.006 (2016).
14 Cruys, B. et al. Glycolytic regulation of cell rearrangement in angiogenesis. Nat Commun 7, 12240, doi:10.1038/ncomms12240 (2016).
15 Schoors, S. et al. Fatty acid carbon is essential for dNTP synthesis in endothelial cells. Nature 520, 192-197, doi:10.1038/nature14362 (2015).
16 Xiong, J. et al. A Metabolic Basis for Endothelial-to-Mesenchymal Transition. Mol Cell 69, 689-698 e687, doi:10.1016/j.molcel.2018.01.010 (2018).
17 Lovisa, S. & Kalluri, R. Fatty Acid Oxidation Regulates the Activation of Endothelial-to-Mesenchymal Transition. Trends Mol Med 24, 432-434, doi:10.1016/j.molmed.2018.03.003 (2018).
18 Zeisberg, M. & Neilson, E. G. Mechanisms of tubulointerstitial fibrosis. Journal of the American Society of Nephrology: JASN 21, 1819-1834, doi:10.1681/ASN.2010080793 (2010).
19 Zeisberg, E. M., Potenta, S. E., Sugimoto, H., Zeisberg, M. & Kalluri, R. Fibroblasts in kidney fibrosis emerge via endothelial-to-mesenchymal transition. J Am Soc Nephrol 19, 2282-2287 (2008).
20 Zeisberg, E. M. et al. Endothelial-to-mesenchymal transition contributes to cardiac fibrosis. Nat Med 13, 952-961 (2007).
21 Li, J. et al. Endothelial FGFR1 (Fibroblast Growth Factor Receptor 1) Deficiency Contributes Differential Fibrogenic Effects in Kidney and Heart of Diabetic Mice. Hypertension 76, 1935-1944, doi:10.1161/HYPERTENSIONAHA.120.15587 (2020).
22 Medici, D. & Kalluri, R. Endothelial-mesenchymal transition and its contribution to the emergence of stem cell phenotype. Semin Cancer Biol 22, 379-384, doi:10.1016/j.semcancer.2012.04.004 (2012).
23 Goodwin, J. E., Feng, Y., Velazquez, H. & Sessa, W. C. Endothelial glucocorticoid receptor is required for protection against sepsis. Proc Natl Acad Sci USA 110, 306-311, doi:10.1073/pnas.1210200110 (2013).
24 Goodwin, J. E., Feng, Y., Velazquez, H., Zhou, H. & Sessa, W. C. Loss of the endothelial glucocorticoid receptor prevents the therapeutic protection afforded by dexamethasone after LPS. PLoS One 9, e108126, doi:10.1371/journal.pone.0108126 (2014).
25 Goodwin, J. E. et al. Endothelial glucocorticoid receptor suppresses atherogenesis—brief report. Arterioscler Thromb Vasc Biol 35, 779-782, doi:10.1161/ATVBAHA.114.304525 (2015).
26 Zhou, H. et al. Loss of the podocyte glucocorticoid receptor exacerbates proteinuria after injury. Sci Rep 7, 9833, doi:10.1038/s41598-017-10490-z (2017).
27 Zhou, H. et al. Endothelial cell-glucocorticoid receptor interactions and regulation of Wnt signaling. JCI Insight 5, doi:10.1172/jci.insight.131384 (2020).
28 He, W. el al. Wnt/beta-catenin signaling promotes renal interstitial fibrosis. Journal of the American Society of Nephrology: JASN 20, 765-776, doi:10.1681/ASN.2008060566 (2009).
29 Kanasaki, K. et al. Linagliptin-Mediated DPP-4 Inhibition Ameliorates Kidney Fibrosis in Streptozotocin-Induced Diabetic Mice by Inhibiting Endothelial-to-Mesenchymal Transition in a Therapeutic Regimen. Diabetes 63, 2120-2131, doi:10.2337/db13-1029 (2014).
30 Srivastava, S. P. et al. Effect of Antifibrotic MicroRNAs Crosstalk on the Action of N-acetyl-seryl-aspartyl-lysyl-proline in Diabetes-related Kidney Fibrosis. Sci Rep 6, 29884, doi:10.1038/srep29884 (2016).
31 Shi, S. et al. Interactions of DPP-4 and integrin beta1 influences endothelial-to-mesenchymal transition. Kidney Int 88, 479-489, doi:10.1038/ki.2015.103 (2015).
32 Srivastava, S. P. et al. SIRT3 deficiency leads to induction of abnormal glycolysis in diabetic kidney with fibrosis. Cell Death Dis 9, 997, doi:10.1038/s41419-018-1057-0 (2018).
33 Sugimoto, H., Grahovac, G., Zeisberg, M. & Kalluri, R. Renal fibrosis and glomerulosclerosis in a new mouse model of diabetic nephropathy and its regression by bone morphogenic protein-7 and advanced glycation end product inhibitors. Diabetes 56, 1825-1833, doi:10.2337/db06-1226 (2007).
34 Fielding, C. A. et al. Interleukin-6 signaling drives fibrosis in unresolved inflammation. Immunity 40, 40-50, doi: 10.1016/j.immuni.2013.10.022 (2014).
35 Apalset, E. M. et al. Interferon gamma (IFN-gamma)-mediated inflammation and the kynurenine pathway in relation to risk of hip fractures: the Hordaland Health Study. Osteoporos Int 25, 2067-2075, doi:10.1007/s00198-014-2720-7 (2014).
36 Fathy, S. A., Mohamed, M. R., Ali, M. A. M., El-Helaly, A. E. & Alattar, A. T. Influence of IL-6, IL-10, IFN-gamma and TNF-alpha genetic variants on susceptibility to diabetic kidney disease in type 2 diabetes mellitus patients. Biomarkers 24, 43-55, doi:10.1080/1354750X.2018.1501761 (2019).
37 Owens, E. P. et al. Biomarkers and the role of mast cells as facilitators of inflammation and fibrosis in chronic kidney disease. Transl Androl Urol 8, S175-S183, doi:10.21037/tau.2018.11.03 (2019).
38 Hickey, F. B. & Martin, F. Role of the Immune System in Diabetic Kidney Disease. Curr Diab Rep 18, 20, doi:10.1007/s11892-018-0984-6 (2018).
39 He, W. et al. Wnt/beta-catenin signaling promotes renal interstitial fibrosis. J Am Soc Nephrol 20, 765-776, doi: 10.1681/ASN.2008060566 (2009).
40 Zhang, L. S. & Lum, L. Delivery of the Porcupine Inhibitor WNT974 in Mice. Methods Mol Biol 1481, 111-117, doi:10.1007/978-1-4939-6393-5_12 (2016).
41 Kang, H. M. et al. Defective fatty acid oxidation in renal tubular epithelial cells has a key role in kidney fibrosis development. Nat Med 21, 37-46, doi:10.1038/nm.3762 (2015).
42 Lovisa, S. et al. Epithelial-to-mesenchymal transition induces cell cycle arrest and parenchymal damage in renal fibrosis. Nat Med 21, 998-1009, doi:10.1038/nm.3902 (2015).
43 Tran, M. T. et al. PGC1alpha drives NAD biosynthesis linking oxidative metabolism to renal protection. Nature 531, 528-532, doi:10.1038/nature17184 (2016).
44 Qi, W. et al. Pyruvate kinase M2 activation may protect against the progression of diabetic glomerular pathology and mitochondrial dysfunction. Nat Med 23, 753-762, doi:10.1038/nm.4328 (2017).
45 Delgado, M. & Ganea, D. Inhibition of endotoxin-induced macrophage chemokine production by VIP and PACAP in vitro and in vivo. Arch Physiol Biochem 109, 377-382, doi:10.1076/apab.109.4.377.4237 (2001).
46 Kato, H. et al. Wnt/beta-catenin pathway in podocytes integrates cell adhesion, differentiation, and survival. J Biol Chem 286, 26003-26015, doi:10.1074/jbc.M111.223164 (2011).
47 Zhou, Z. et a. MicroRNA-27a promotes podocyte injury via PPARgamma-mediated beta-catenin activation in diabetic nephropathy. Cell Death Dis 8, e2658, doi:10.1038/cddis.2017.74 (2017).
48 Feng, Y., Liang, Y., Ren, J. & Dai, C. Canonical Wnt Signaling Promotes Macrophage Proliferation during Kidney Fibrosis. Kidney Dis (Basel) 4, 95-103, doi:10.1159/000488984 (2018).
49 Wanner, C., Drechsler, C. & Krane, V. Lipid metabolism in chronic kidney disease: the role of statins in cardiovascular risk. J Ren Nutr 17, 75-78, doi:10.1053/j.jrn.2006.10.012 (2007).
50 Srivastava, S. P., Shi, S., Koya, D. & Kanasaki, K. Lipid mediators in diabetic nephropathy. Fibrogenesis Tissue Repair 7, 12, doi:10.1186/1755-1536-7-12 (2014).
51 Herman-Edelstein, M., Scherzer, P., Tobar, A., Levi, M. & Gafter, U. Altered renal lipid metabolism and renal lipid accumulation in human diabetic nephropathy. J Lipid Res 55, 561-572, doi:10.1194/jlr.P040501 (2014).
52 Wahl, P., Ducasa, G. M. & Fornoni, A. Systemic and renal lipids in kidney disease development and progression. Am J Physiol Renal Physiol 310, F433-445, doi:10.1152/ajprenal.00375.2015 (2016).
53 Kovesdy, C. P., Anderson, J. E. & Kalantar-Zadeh, K. Inverse association between lipid levels and mortality in men with chronic kidney disease who are not yet on dialysis: effects of case mix and the malnutrition-inflammation-cachexia syndrome. J Am Soc Nephrol 18, 304-311, doi:10.1681/ASN.2006060674 (2007).
54 Diamond, J. R. Analogous pathobiologic mechanisms in glomerulosclerosis and atherosclerosis. Kidney Int Suppl 31, S29-34 (1991).
55 Kwan, B. C., Kronenberg, F., Beddhu, S. & Cheung, A. K. Lipoprotein metabolism and lipid management in chronic kidney disease. J Am Soc Nephrol 18, 1246-1261, doi:10.1681/ASN.2006091006 (2007).
56 Baigent, C. et al. First United Kingdom Heart and Renal Protection (UK-HARP-I) study: biochemical efficacy and safety of simvastatin and safety of low-dose aspirin in chronic kidney disease. Am J Kidney Dis 45, 473-484, doi:10.1053/j.ajkd.2004.11.015 (2005).
57 Sharp Collaborative, G. Study of Heart and Renal Protection (SHARP): randomized trial to assess the effects of lowering low-density lipoprotein cholesterol among 9,438 patients with chronic kidney disease. Am Heart J 160, 785-794 e710, doi:10.1016/j.ahj.2010.08.012 (2010).
58 Kimura, S. et al. Randomized comparison of pitavastatin and pravastatin treatment on the reduction of urinary albumin in patients with type 2 diabetic nephropathy. Diabetes Obes Metab 14, 666-669, doi:10.1111/j.1463-1326.2012.01566.x (2012).
59 Abe, M. et al. Effects of lipid-lowering therapy with rosuvastatin on kidney function and oxidative stress in patients with diabetic nephropathy. J Atheroscler Thromb 18, 1018-1028, doi:10.5551/jat.9084 (2011).
60 Rutter, M. K. et al. Protection Against Nephropathy in Diabetes with Atorvastatin (PANDA): a randomized double-blind placebo-controlled trial of high-vs. low-dose atorvastatin(1). Diabet Med 28, 100-108, doi:10.1111/j.1464-5491.2010.03139.x (2011).
61 Colhoun, H. M. et al. Effects of atorvastatin on kidney outcomes and cardiovascular disease in patients with diabetes: an analysis from the Collaborative Atorvastatin Diabetes Study (CARDS). Am J Kidney Dis 54, 810-819, doi:10.1053/j.ajkd.2009.03.022 (2009).
62 Coonrod, B. A. et al. Predictors of microalbuminuria in individuals with IDDM. Pittsburgh Epidemiology of Diabetes Complications Study. Diabetes Care 16, 1376-1383, doi:10.2337/diacare.16.10.1376 (1993).
63 Ting, R. D. et al. Benefits and safety of long-term fenofibrate therapy in people with type 2 diabetes and renal impairment: the FIELD Study. Diabetes Care 35, 218-225, doi:10.2337/dc11-1109 (2012).
64 Li, J. et al. FGFR1 is critical for the anti-endothelial mesenchymal transition effect of N-acetyl-seryl-aspartyl-lysyl-proline via induction of the MAP4K4 pathway. Cell Death Dis 8, e2965, doi:10.1038/cddis.2017.353 (2017).
65 Price, N. L. et al. Genetic deficiency or pharmacological inhibition of miR-33 protects from kidney fibrosis. JCI Insight 4, doi:10.1172/jci.insight.131102 (2019).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for treating a condition or a disease in a subject in need thereof, which method comprises administering to the subject a therapeutically effective amount of an inhibitor of IL-6 activity, wherein the condition or disease is selected from organ fibrosis, nephropathy, dyslipidemia, hypertension, hyperlipidemia, hypercholesterolemia, cardiovascular disease, peripheral artery disease, atherosclerosis, coronary artery disease, coronary heart disease, and stroke.

2. The method of claim 1, wherein the organ fibrosis is renal fibrosis.

3. The method of claim 1, wherein nephropathy is diabetic nephropathy.

4. The method of claim 1, wherein the inhibitor of IL-6 activity is an antibody or an antigen-binding fragment of an antibody.

5. The method of claim 4, wherein the inhibitor is an IL-6 neutralizing antibody or an antigen-binding fragment thereof.

6. The method of claim 4, wherein the inhibitor is sirukumab, siltuximab, or olokizumab or an antigen-binding fragment thereof.

7. The method of claim 4, wherein the inhibitor is an antibody or an antigen-binding fragment which targets IL-6 receptor.

8. The method of claim 7, wherein the inhibitor is tocilizumab or sarilumab or or an antigen-binding fragment thereof.

9. The method of claim 1, wherein the inhibitor of IL-6 activity provides trans-signaling blockade.

10. The method of claim 9, wherein the inhibitor is olamkicept (FE999301), or a fragment or variant thereof.

11. The method of claim 1, wherein the inhibitor of IL-6 activity is an inhibitor of intracellular signaling.

12. The method of claim 11, wherein the inhibitor is tofacitinib or CpG-stat3 siRNA.

13. The method of claim 1, further comprising administering to the subject one or more additional agents effective to treat said condition or disease.

14. The method of claim 13, wherein the one or more additional agents comprise one or more cholesterol-lowering drugs, blood pressure-lowering therapies, antiinflammatory agents, anti-thrombotic agents, anti-coagulant agents, inhibitors of the renin-angiotensin aldosterone system (RAAS inhibitors), beta-adrenergic blockers, calcium channel blockers, blood sugar reducing medications, and/or Wnt inhibitors.

15. The method of claim 14, wherein the cholesterol-lowering drugs comprise statins, fibrates, and/or inhibitors of proprotein convertase subtilisin/kexin type 9.

16. The method of claim 14, wherein the blood pressure-lowering therapies comprise angiotensin-converting enzyme (ACE) inhibitors and/or angiotensin II receptor blockers (ARBs).

17. The method of claim 14, wherein the blood sugar reducing medications comprise metformin, insulin, glucose-dependent insulinotropic polypeptide (GIP), and glucagon-like peptide 1 (GLP-1), and/or sodium/glucose co-transporter 2 (SGLT2) inhibitors.

18. The method of claim 14, wherein the Wnt inhibitor comprise a compound having the structure according to formula (I): 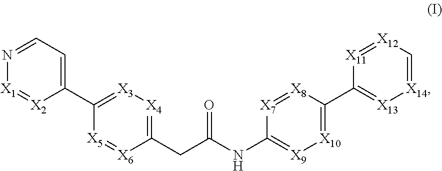
wherein X₁ and X₂ are selected from N and CR;
one of X₃, X₄, X₅ and X₆ is N and others are selected from N and CR;
one of X₇, X₈, X₉ and X₁₀ is N and others are selected from N and CR;
one of X₁₁, X₁₂, X₁₃ and X₁₄ is N and others are selected from N and CR, and
R is independently at each occurrence selected from hydrogen, halo, cyano, methyl, difluoromethyl, and trifluoromethyl,
or a pharmaceutically acceptable salt thereof.

19. The method of claim 1, wherein the subject is human.


SEQUENCE LISTING (26 sequences)

SEQ ID NO: 1 - DNA, 20 bp, synthetic construct
  source 1..20: mol_type = other DNA
aggcttgcatagagctccag

SEQ ID NO: 2 - DNA, 20 bp, synthetic construct
  source 1..20: mol_type = other DNA
ctgacagaggcaccactgaa

SEQ ID NO: 3 - DNA, 21 bp, synthetic construct
  source 1..21: mol_type = other DNA
aacctatgcccgtttcctcta

SEQ ID NO: 4 - DNA, 24 bp, synthetic construct
  source 1..24: mol_type = other DNA
ggtggccgaatgcacattgaaaga

SEQ ID NO: 5 - DNA, 20 bp, synthetic construct
  source 1..20: mol_type = other DNA
cgaggtgacagagaccacaa

SEQ ID NO: 6 - DNA, 20 bp, synthetic construct
  source 1..20: mol_type = other DNA
ccaagcaacgacaaaatacc

SEQ ID NO: 7 - DNA, 20 bp, synthetic construct
  source 1..20: mol_type = other DNA
tctgaaggactctggctttg

SEQ ID NO: 8 - DNA, 20 bp, synthetic construct
  source 1..20: mol_type = other DNA
cacaaagcagccttgcagaa

SEQ ID NO: 9 - DNA, 19 bp, synthetic construct
  source 1..19: mol_type = other DNA
gctccagaaggccctcaga

SEQ ID NO: 10 - DNA, 21 bp, synthetic construct
  source 1..21: mol_type = other DNA
ctcagccctgatgcttctcac

SEQ ID NO: 11 - DNA, 20 bp, synthetic construct
  source 1..20: mol_type = other DNA
gctgctttgcctacctctcc

SEQ ID NO: 12 - DNA, 20 bp, synthetic construct
  source 1..20: mol_type = other DNA
agctagtcgggagagcctac

SEQ ID NO: 13 - DNA, 22 bp, synthetic construct
  source 1..22: mol_type = other DNA
ttccgataacgaacgagactct

SEQ ID NO: 14 - DNA, 20 bp, synthetic construct
  source 1..20: mol_type = other DNA
ttcttggtttccagctatgg

SEQ ID NO: 15 - DNA, 17 bp, synthetic construct
  source 1..17: mol_type = other DNA
gaaatagccaagctcag

SEQ ID NO: 16 - DNA, 22 bp, synthetic construct
  source 1..22: mol_type = other DNA
gagtgtaaagacttggtccacc

SEQ ID NO: 17 - DNA, 22 bp, synthetic construct
  source 1..22: mol_type = other DNA
tttgcctgttcttccctggaca

SEQ ID NO: 18 - DNA, 20 bp, synthetic construct
  source 1..20: mol_type = other DNA
ctggagtcaagccagacaca

SEQ ID NO: 19 - DNA, 22 bp, synthetic construct
  source 1..22: mol_type = other DNA
gttgaagacaaaccgtttttcc

SEQ ID NO: 20 - DNA, 21 bp, synthetic construct
  source 1..21: mol_type = other DNA
gatggatgctaccaaactgga

SEQ ID NO: 21 - DNA, 21 bp, synthetic construct
  source 1..21: mol_type = other DNA
agagcaggcagcatagcagtg

SEQ ID NO: 22 - DNA, 19 bp, synthetic construct
  source 1..19: mol_type = other DNA
ctttccctccgcattgaca

SEQ ID NO: 23 - DNA, 21 bp, synthetic construct
  source 1..21: mol_type = other DNA
agaggggcaggaaatctgaac

SEQ ID NO: 24 - DNA, 21 bp, synthetic construct
  source 1..21: mol_type = other DNA
tcgagtgacaaacacgactgc

SEQ ID NO: 25 - DNA, 20 bp, synthetic construct
  source 1..20: mol_type = other DNA
aaggaagtgaccgtgagcag

SEQ ID NO: 26 - DNA, 18 bp, synthetic construct
  source 1..18: mol_type = other DNA
ggctgaacgccacttgtc